(19) United States
(12) Patent Application Publication
(10) Pub. No.: US 2004/0043420 A1
(43) Pub. Date: Mar. 4, 2004

(54) Method of identifying conformation-sensitive binding peptides and uses thereof

(76) Inventors: Dana Fowlkes, North Carolina, CA (US); Thomas R. Barnett, North Carolina, CA (US); Benjamin Buehrer, Chapel Hill, NC (US)

Correspondence Address: BROWDY AND NEIMARK, P.L.L.C., 624 NINTH STREET, NW, SUITE 300, WASHINGTON, DC, 20001-5303, US

(21) Appl. No.: 10/332,708

(22) PCT Filed: Jul. 11, 2001
(86) PCT No.: PCT/US01/21867

(51) Int. Cl. G01N 33/53; G01N 33/567

(52) U.S. Cl. 435/7.1; 435/7.2

(57) ABSTRACT

Peptides which bind a cellular (surface or intracellular) receptor, such as a nuclear receptor, may be identified by screening a combinatorial peptide library presented in the form of cells each of which coexpress one member peptide and the receptor, together with a signal producing system for reporting binding. A “two-hybrid” assay is of particular interest. The screen may be carried out in the presence of a ligand, in particular, an exogenous ligand. If this screening is carried out for a plurality of different receptor conformations, then this library screening will also serve to identify conformation-specific peptides for the receptor, which may then be used in a panel for “fingerprinting” query compounds as to their ability to interact with the receptor in the presence of each of the panel peptides. These fingerprints may be compared to those of reference compounds with known biological activities mediated by that receptor.

This application is a continuation-in-part of Ser. No. 09/860,688, filed May 21, 2001, which is a continuation-in-part of Ser. No. 09/614,865, filed Jul. 12, 2000, all hereby incorporated by reference.

### CROSS-REFERENCE TO RELATED APPLICATIONS

Paige, et al., Ser. No. 09/429,331, filed Oct. 28, 1999, which is a continuation-in-part of Paige, et al., PCT/US99/06664, filed Mar. 26, 1999, which is a nonprovisional of (1) No. 60/115,345, filed Jan. 8, 1999, (2) Paige, et al., Serial No. 60/099,656, filed Sep. 9, 1998, and (3) Paige, et al., Serial No. 60/082,756, filed Apr. 23, 1998, all hereby incorporated-by-reference, relate to in vitro and in vivo methods of screening compounds for biological activity.

Thorp, Ser. No. 08/904,842, METHOD OF IDENTIFYING AND DEVELOPING DRUG LEADS WHICH MODULATE THE ACTIVITY OF A TARGET PROTEIN, discloses several methods of identifying drug leads. In essence a protein of interest, in one or more states, is characterized by (a) its chemical reactivity with one or more characterizing reagents, and/or (b) its binding to one or more aptamers (especially nucleic acids), generating an array of descriptors by which it may be characterized as more or less similar for reference proteins for which an equivalent array of descriptors have been generated, and for which one or more activity-mediating reference drugs are known. Suitable drug leads for the protein of interest are those analogous to the reference drugs for the more similar reference proteins.

Fowlkes, et al. PCT/US97/19638, Ser. Nos. 08/740,671, 09/050,359 and 09/069,827, IDENTIFICATION OF DRUGS USING COMPLEMENTARY COMBINATORIAL LIBRARIES, disclose the use of a first combinatorial library, e.g., of peptides, to obtain a set of binding peptides that can serve as a surrogate for the natural ligand of a target protein. A small organic compound library (preferably combinatorial in nature) is then screened for compounds which inhibit the binding of the surrogates to the target protein.

All of the above applications are hereby incorporated by reference.

### BACKGROUND OF THE INVENTION

1. Field of the Invention

This invention relates to a method of identifying drugs which can mediate the biological activity of a target protein. It also relates to reagents, especially peptides, useful in that method, or more directly in mediating the biological activity of said target protein or in binding to said target protein themselves.

2. Description of the Background Art

Protein Binding and Biological Activity

Many of the biological activities of the proteins are attributable to their ability to bind specifically to one or more binding partners (ligands), which may themselves be proteins, or other biomolecules.

When the binding partner of a protein is known, it is relatively straightforward to study how the interaction of the binding protein and its binding partner affects biological activity. Moreover, one may screen compounds for the ability of the compound to competitively inhibit the formation of the complex, or to dissociate an already formed complex. Such inhibitors are likely to affect the biological activity of the protein, at least-if they can be delivered in vivo to the site of the interaction.

If the binding protein is a receptor, and the binding partner an effector of the biological activity, then the inhibitor will antagonize the biological activity. If the binding partner is one which, through binding, blocks a biological activity, then an inhibitor of that interaction will, in effect, be an agonist.

Screening for Modulators of Receptor Activity

The current state of the art for screening for modulators of receptor activity involves the displacement of a labeled ligand from the ligand binding pocket of the receptor. For example, a screen may be for displacement of radiolabeled estradiol from the estrogen receptor. This assay only provides information concerning the relative affinities of the compounds for the receptor and gives no indication of the activity of the compound on the receptor, that is whether it functions as an agonist or an antagonist of receptor activity. This is a major problem for pharmaceutical companies to overcome in screening for modulators of receptor activity.

The assays that have been developed to date that can distinguish between agonists and antagonists involve cell-based assays and reporter gene systems. McDonnell, et al., Molec. Endocrinol., 9:659 (1995). In these systems, the receptor and a reporter gene are co-transfected into cells in culture. The reporter gene is only activated in the presence of active receptor. The ability of a compound to modulate receptor activity is determined by the relative strength of the reporter gene activity. These assays are time consuming and can produce variable results in different cell lines or with different reporter genes or response elements. Thus, the data must be interpreted with caution.

Methods have been developed that also take advantage of the different conformational states of receptors. Proteolytic digestion of the estrogen receptor in the presence of an agonist or antagonist produces distinct banding patterns on a denaturing polyacrylamide gel. In certain conformations, the receptor is protected from digestion at a particular site, while a different conformation may expose that site. Thus the banding patterns may indicate whether the receptor was complexed with an agonist or antagonist at the time of proteolytic digestion. This method requires copious amounts of receptor protein and is time consuming and expensive in that it requires a gel to be run for each sample. It is not suitable for screening numerous samples.

The following are examples of patents on cell based screening methods:

U.S. Pat. No. 5,723,291—Methods for screening compounds for estrogenic activity

U.S. Pat. No. 5,298,429—Bioassay for identifying ligands for steroid hormone receptors

U.S. Pat. No. 5,445,941—Method for screening anti-osteoporosis agents

U.S. Pat. No. 5,071,773—Hormone receptor-related bioassays

U.S. Pat. No. 5,217,867—Receptors: their identification, characterization, preparation and use

Traditional Drug Screening

In traditional drug screening, natural products (especially those used in folk remedies) were tested for biological activity. The active ingredients of these products were purified and characterized, and then synthetic analogues of these “drug leads” were designed, prepared and tested for activity. The best of these analogues became the next generation of “drug leads”, and new analogs were made and evaluated.

Both natural products and synthetic compounds could be tested for just a single activity, or tested exhaustively for any biological activity of the interest to the tester. Testing was originally carried out in animals, later, less expensive and more convenient model systems, employing isolated organs, tissue, or cells, or cell cultures, membrane extracts or purified receptors, were developed for some pharmacological evaluations.

Testing in whole animals and isolated organs typically requires large amounts of chemical compound to test. Since the quantity of a given compound within a collection of potential medicinal compounds is limited, this requires one to limit the number of screens executed.

Also, it is inherently difficult to establish structure/activity relationships (SAR) among compounds tested using whole animals, or isolated organs or tissues or, to a lesser extent, cultured cells. This is because the actual molecular target of any given compound's action may be quite different from that of other compounds scoring positive in the assay. By testing a battery of compounds on a very specific target, one can correlate the action of various chemical residues with the quantitative activity and use that information to focus ones search for active compounds among certain classes of compounds or even direct the synthesis of novel compounds having a composite of the properties shared by the active compounds tested.

Another disadvantage to whole animal, organ, tissue and cell based screening is that certain limitations may prevent an active compound from being scored as such. For instance, an inability to pass through the cellular membrane may prevent a potent inhibitor, within a tested compound library, from acting on the activated oncogene ras and giving a spurious negative score in a cell proliferation assay. However, if it were possible to test ras in an isolated system, that potent inhibitor would be scored as a positive compound and contribute to the establishment of a relevant SAR. Subsequent, chemical modifications could then be carried out to optimize the compound structure for membrane permeability. (In the case of cell-based assays, this problem can be alleviated to some degree by altering membrane permeability.)

Drug Discovery. The human genomics effort could yield gene sequences that code for as many as 70,000 proteins, each a potential drug target; microbial genomics will increase this number further. Unfortunately, since genomic studies identify genes, but not the biological activity of the corresponding proteins, it is likely that many of the genes will prove to encode proteins-whose activation or inactivation has no effect on disease progression. (Gold, et al., J. Nature Biotech., 15:297, 1997). There is therefore a need for a method of determining which proteins are most likely to be productive targets for pharmacological intervention.

Even if one knew in advance the perhaps 10,000 proteins which could be considered interesting targets, there remains the problem of efficiently screening hundreds of thousands of possible drugs for a useful activity against these 10,000 targets.

Historically, acquiring chemical compound libraries has been a barrier to the entry of smaller firms into the drug discovery arena. Due to the large quantity of chemical required for testing on whole animals and even on cells in culture, it was a given that whenever a compound was synthesized it should be done in fairly large quantity. Thus, there was a synthesis and purification throughput of less than 50 compounds per chemist per year. Large companies maintained their immensely valuable collections as trade barriers. However, with the downsizing of targets to the molecular level and the automation of screens, the quantity of a given compound necessary for an assay has been reduced to very small amounts. These changes have opened the door for the utilization of so-called combinatorial chemistry libraries in lieu of the traditional chemical libraries. Combinatorial chemistry permits the rapid and relatively inexpensive synthesis of large numbers of compounds in the small quantities suitable for automated assays directed at molecular targets. Numerous small companies and academic laboratories have successfully engineered combinatorial chemical libraries with a significant range of diversity (reviewed in Doyle, 1995, Gordon et al, 1994a, Gordon et al, 1994b).

Combinatorial Libraries. In a combinatorial library, chemical building blocks are randomly combined into a large number (as high as 10E15) of different compounds, which are then simultaneously screened for binding (or other) activity against one or more targets.

Libraries of thousands, even millions, of random oligopeptides have been prepared by chemical synthesis (Houghten et al., Nature, 354:84-6(1991)), or gene expression (Marks et al., J Mol Biol, 222:581-97(1991)), displayed on chromatographic supports (Lam et al., Nature, 354:82-4(1991)), inside bacterial cells (Colas et al., Nature, 380:548-550(1996)), on bacterial pili (Lu, Bio/Technology, 13:366-372(1990)), or phage (Smith, Science, 228:1315-7(1985)), and screened for binding to a variety of targets including antibodies (Valadon et al., J Mol Biol, 261:11-22(1996)), cellular proteins (Schmitz et al., J Mol Biol, 260:664-677(1996)), viral proteins (Hong and-Boulanger, Embo J, 14:4714-4727(1995)), bacterial proteins (Jacobsson and Frykberg, Biotechniques, 18:878-885(1995)), nucleic acids (Cheng et al., Gene, 171:1-8(1996)), and plastic (Siani et al., J Chem Inf Comput Sci, 34:588-593(1994)).

Libraries of proteins (Ladner, U.S. Pat. No. 4,664,989), peptoids (Simon et al., Proc Natl Acad Sci USA, 89:9367-71(1992)), nucleic acids (Ellington and Szostak, Nature, 246:818(1990)), carbohydrates, and small organic molecules (Eichler et al., Med Res Rev, 15:481-96(1995)) have also been prepared or suggested for drug screening purposes.

The first combinatorial libraries were composed of peptides or proteins, in which all or selected amino acid positions were randomized. Peptides and proteins can exhibit high and specific binding activity, and can act as catalysts. In consequence, they are of great importance in biological systems. Unfortunately, peptides per se have limited utility for use as therapeutic entities. They are costly to synthesize, unstable in the presence of proteases and in general do not transit cellular membranes. Other classes of compounds have better properties for drug candidates.

Nucleic acids have also been used in combinatorial libraries. Their great advantage is the ease with which a nucleic acid with appropriate binding activity can be amplified. As a result, combinatorial libraries composed of nucleic acids can be of low redundancy and hence, of high diversity. However, the resulting oligonucleotides are not suitable as drugs for several reasons. First, the oligonucleotides have high molecular weights and cannot be synthesized conveniently in large quantities. Second, because oligonucleotides are polyanions, they do not cross cell membranes. Finally, deoxy- and ribo-nucleotides are hydrolytically digested by nucleases that occur in all living systems and are therefore usually decomposed before reaching the target.

There has therefore been much interest in combinatorial libraries based on small molecules, which are more suited to pharmaceutical use, especially those which, like benzodiazepines, belong to a chemical class which has already yielded useful pharmacological agents. The techniques of combinatorial chemistry have been recognized as the most efficient means for finding small molecules that act on these targets. At present, small molecule combinatorial chemistry involves the synthesis of either pooled or discrete molecules that present varying arrays of functionality on a common scaffold. These compounds are grouped in libraries that are then screened against the target of interest either for binding or for inhibition of biological activity. Libraries containing hundreds of thousands of compounds are now being routinely synthesized; however, screening these large libraries for binding or inhibition with all 10,000 potential targets cannot be reasonably accomplished with present screening technologies, and there are numerous experimental and computational strategies under development to reduce the number of compounds that must be screened for each target.

Information-intensive drug discovery. As pointed out by Paterson, et al., J. Med. Chem., 39: 3049-59 (1996), medicinal chemistry advances through the dual processes of “lead discovery” and “lead optimization”. In “lead discovery”, the search objective is the discovery of an “activity island”, a chemical class with a high frequency of active molecules. (this class may be defined mathematically as a volume within a multidimensional space defined by various molecular descriptors). In “lead optimization”, the “activity island” is explored in detail. If each compound synthesized and tested can be considered as a probe of a “neighborhood” of similar compounds, in “lead discovery”, it is inefficient to test substances whose neighborhoods overlap.

Coupled to the recent advancements in genomics and molecular biology has been a revolution in information technology, which includes relational databases, computer graphics, and neural networks. These capabilities permit the construction of databases of descriptors that describe either compounds or targets in quantitative terms, and these descriptors can be related to make predictions about the structures of compounds, their biological activities, and the targets they act on.

Structure descriptors can be based on a variety of structural features. These approaches provide arrays of molecular descriptors that can be used to assess the similarity of molecules in a library.

See Patterson, et al., et al., J. Med. Chem., 39: 3049-59 (1996), Klebe and Abraham, J. Med. Chem., 36:70-80 (1993), Cummins, et al., J. Chem. Inf. Comput. Sci., 36:750-63 (1996), Matter, J. Med. Chem., 40:1219-29 (1997); Weinstein, et al., Science, 275:343-9 (1997).

For proteins, structural descriptors cannot be directly calculated from the amino acid sequence.

Compounds may be characterized by their activity rather than by structure. Kauvar, et al., Chemistry & Biology, 2: 107-118 (1995) “fingerprinted” over 5,000 compounds by the binding potency (concentration needed to inhibit 50% of the protein's activity) of each compound to each member of a reference panel of eight proteins. (These proteins were selected on the basis of readily assayable activity, broad cross-reactivity with small organic molecules, and low correlation between each other in binding patterns.) A screening library of 54 compounds was then selected based on the diversity in their “fingerprints” (inhibitory activity against the reference panel proteins).

This “training set” was used to evaluate the similarity of the ligand binding characteristics of a new protein to one of the reference panel proteins. By regression analysis, a computational surrogate (a weighted sum of two or more reference panel proteins) for the new protein is determined. The activity of all fingerprinted compounds to inhibit the activity of the new protein is predicted as the sum of their appropriately weighted inhibitory activities against the component reference proteins of the computational surrogate. Predictions may be improved by testing additional sets of compounds against the new protein. See also L. M. Kauvar, H. O. Villar. Method to identify binding partners. U.S. Pat. No. 5,587,293.

Weinstein, supra, in a study of the molecular pharmacology of cancer, took a similar approach. The “activity” database (A) contains the activities against 60 cell lines for 60,000 compounds that have been screened at NCI. The similarity in the activity profile against the panel of cell lines can then be calculated for any two compounds, and is generally assessed by a pairwise correlation coefficient (PCC), which is determined by an algorithm called COMPARE, which calculates the similarity of all of the compounds in the database to a user-supplied “seed” compound.

High-Throughput Screening

A high-throughput screening system usually comprises (1) suitably arrayed compound libraries, (2) an assay method configured for automation, (3) a robotics workstation for performing the method, and (4) a computerized system for handling the data.

The array may be a standard 96-well microtitre plate, or an array of compounds on chips, beads, agar plates or other solid support. The array may be a simplex array of individual compounds or a complex array in which each element is a predetermined mixture of a small number, e.g., 10-20, different compounds. In the latter case, the mixture ultimately must be deconvolved to identify the true active component(s).

For ease of automation, the assay should require as few steps as possible. Thus, homogeneous assays, which do not require fractionations, or more than a single addition of reagent, are desirable.

See generally Broach and Thorner, Nature, 384, 14 (Nov. 7, 1996); Milligan and Rees, Trends Pharmacol. Sci., 20:118-24 (1999).

Preferred reporter genes for high-throughput screening include bacterial beta-galactosidase, luciferase, human placental alkaline phosphatase, bacterial beta-lactamase, and jellyfish green fluorescent protein.

Gonzalez and Negulescu, Curr. Op. Biotechnol., 9:624-31 (1998), discuss intracellular detection assays suitable for high-throughput screening. Such assays are conveniently provided as optical assays, which may rely on absorbance, fluorescence, or luminescence as readouts. While absorbance assays ave been useful in melanophore and beta-galactosidase reporter assays for GPCRs, such assays have relatively low sensitivity. To achieve significant absorbance changes, very high concentrations of dyes and many cells are necessary. Hence, the absorbance assays do not lend themselves as well to miniaturized formats.

In contrast, luminescence and fluorescence are more sensitive and high S/N ratios are commonplace.

With regard to chemiluminescence assays, the standard substrates are luciferin and aequorin. Since high concentrations of luciferin and ATP are desirable to drive luciferase-catalyzed reactions, the luciferase assay is usually conducted in cell lysates from thousands of cells, rather than in intact cells. Membrane-impermable luminescent substrates have been used in connection with extracellular or lysate assays. The greatest advantage of chemiluminescence assays is their extremely low background.

Fluorescence can easily be detected at the single cell level. However, the process of exciting fluorescence is not absolutely selective; there is a background of unwanted fluorescence and light scattering from endogenous cellular and equipment sources.

Cell-based fluorescence assays fall into three broad categories: (1) those based on changes in fluorescence intensity, such as those based on the calcium-sensitive Fluo-3 sensor; (2) those based on energy transfer, such as FRET (where there is an energy transfer from a donor fluorophore to an acceptor fluorophore when they are in close proximity and have a spectral overlap); and (3) those based on energy redistribution (where a tagged molecule moves within a cell, and the change in position of the fluorescence within the individual cell is observed).

The possible signals include Ca, cAMP, voltage, enzymatic, protein interaction, and transcription. Ca and cAMP are both mentioned in the context of GPCR targets. For Ca, the suggested readout is Ca indicator dye (fluorescence), Ca photoprotein (luminescence), a reporter gene (fluorescence or luminescence), and cameleon (FRET). For cAMP, the suggested readouts are FlchR (FRET) and a reporter gene (fluorescence or luminescence).

The authors also comment that other detection methods, such as fluorescent polarization, fluorescence correlation spectroscopy, and time-resolved detection, which are still primarily used in biochemical or binding assays, will also undoubtedly migrate into cell based assays.

Cell-Based Assays

Cell-based assays, and in particular the “two-hybrid” assay system, have been used to examine protein: protein interactions, see Fields and Song, Nature, 340:245-6 (1989) and Gyuris, et al., Cell, 75:791-803 (1993), and protein:peptide interactions, see Colas, et al., Nature, 380 (6574):548-50 (1996); Yang, et al., Nucleic Acids Res., 23(7):1152-6 (1995); Kolonin and Finley, Proc. Nat. Acad. Sci. (USA), 95(24):14266-271 (1998); Cohen, et al., Id., 95(24):14272-7 (1998); Geyer, et al., Id., 96(15):8567-72 (1999); Norman, et al., Science, 285 (5427):591-5 (1999); Chang, et al., Mol. Cell. Biol., 19(12):8226-39 (1999). In Yang, a yeast two-hybrid system was used to screen an unbiased combinatorial library of random peptides (16 Xaa positions) to identify peptides which bind the retinoblastoma protein (Rb). Similarly, Cohen used a two-hybrid system to screen a combinatorial peptide library for peptides which inhibit the kinase activity of cyclin-dependent kinase 2 (Cdk2); Cohen notes that this approach preselects for library members which are stable inside cells. The use of two-hybrid assay to screen a combinatorial library is also described by Colas and by Geyer.

There has been no prior use of cell-based assays to screen combinatorial peptide libraries for peptides which bind cellular receptors in a receptor conformation-sensitive manner, or to screen such a library for peptides which bind cellular receptors in the presence of exogenously added ligands, or to screen such a library for peptides which bind a nuclear receptor.

All references, including any patents or patent applications, cited in this specification are hereby incorporated by reference. No admission is made that any reference constitutes prior art. The discussion of the references states what their authors assert and applicants reserve the right to challenge the accuracy and pertinency of the cited document.

### SUMMARY OF THE INVENTION

The present invention relates to cell-based assays for the screening of combinatorial libraries for library members which bind to a target molecule. Structurally speaking, the target molecule is preferably a protein. Functionally speaking, the target molecule is preferably a receptor. The discussion of assays for binding to receptors applies, mutatis mutandis, to other molecules. A target receptor may be endogenous or exogenous to the cell in question. Nuclear receptors, such as the estrogen receptor, are of particular interest.

The present invention also relates to the subsequent identification of the receptor-binding library members which bind in a manner sensitive to receptor conformation, and to the subsequent use of these members (“Biokeys”) in the prediction of the ability of small organic molecules, suitable for pharmaceutical use, to interact with the same receptor.

The receptor-binding library members, and mutants, peptidomimetics and analogues, may also be used in their own right as therapeutic or diagnostic agents.

In a major preferred screening embodiment, the invention relates to a method of identifying a binding peptide which binds a receptor, where said binding peptide is a member of a combinatorial library of peptides and said library is screened for the ability of its members to bind said receptor, in which

said receptor is a surface or intracellular receptor of a cell,

said library is expressed in a plurality of cells, each cell coexpressing said receptor, or a ligand-binding receptor moiety thereof, and one member of said library, said cells collectively expressing all members of said library, each cell further providing a signal producing system operably associated with said receptor or moiety such that a signal is produced which is indicative of whether said member binds said receptor or moiety in or on said cell,

said cells, when screened, are not integrated into a whole multicellular organism or a tissue or organ of such an organism,

where said peptides of said library are screened in a first screening when said receptor is in a first conformation, and one or more of the peptides of said library are screened in a second screening for binding to said receptor in a second and different conformation, said second screening being simultaneous with or subsequent to the first screening.

whereby peptides whose binding to the receptor is receptor conformation-sensitive are identified.

The second screening may be a screening of the entire library screened in the first screening, in which case the screenings will usually be simultaneous. It may be a screening of a subset of that first library. Or it may be a screening of a second library which overlaps with the first library, although this is less preferred. The screened cells are preferably eukaryotic, more preferably yeast cells, for at least the first screening.

In an especially preferred embodiment, said signal producing system comprises (1) a receptor-bound component which is fused to said receptor or moiety so as to provide a chimeric receptor, and (2) a peptide-bound component which is fused to said peptide so as to provide a chimeric peptide, whereby a signal is produced when the peptide-bound and receptor-bound components are brought into physical proximity as a result of the binding of the peptide to the receptor.

In a sub-embodiment of interest, the screened cells are diploid yeast cells, and the diploid yeast cells are obtained by mating haploid cells of a first diploid mating type strain which express the peptide-bound component and haploid cells of a different mating type which express the receptor-bound component.

Another preferred aspect of the invention relates to method of predicting the receptor-modulating activity of a compound which modulates the biological activity of a receptor which comprises:

(I) identifying peptides which bind said receptor, said peptides differing in their ability to bind to said receptor depending on which of a plurality of different reference conformations the receptor is in, at least one of such peptides being identified by the major preferred screening embodiment described above, and

(II) using a plurality of said peptides to predict the receptor-modulating activity of a compound, by

(a) providing a panel comprising a plurality of members, said members including peptides identified in (I) above, said members differing in their ability to bind to said receptor depending on which of a plurality of different reference conformations the receptor is in, where the effect of a plurality of reference substances, known to modulate the biological activity of the receptor, on the binding of each member of the panel is known, and is characterized as a reference fingerprint for each such reference substance;

(b) screening a test substance of unknown activity relative to said receptor to determine its effect on the binding of each member of said panel to said receptor, thereby obtaining a test fingerprint for said test substance,

(c) comparing the test fingerprint to the reference fingerprints, and

(d) predicting the biological activity of the test substance, based on the assumption that its biological activity will be similar to that of reference substances with similar fingerprints.

The screening step (b) may be in vivo or in vitro.

It is particularly desirable that the effect of reference substances on the binding by said panel members is determined by

(a) providing a panel comprising a plurality of members, said members differing in their ability to bind to said receptor depending on which of a plurality of different reference conformations the receptor is in, and

(b) screening a plurality of reference substances known to modulate the biological activity of said receptor to determine their effect on the binding of each member of said panel to said receptor, thereby obtaining a reference fingerprint for each reference substance, said fingerprint comprising a plurality of panel-based descriptors, each panel-based descriptor characterizing the effect of the reference substance on the binding of a particular panel member to said receptor, said reference fingerprint's panel based descriptors collectively characterizing the effect of the reference substance on the binding of all of the panel members, individually, to said receptor.

The panel members generally may be obtained by (a) providing one or more ligands for the receptor; (b) screening a first combinatorial library comprising a plurality of members for the ability to bind to a receptor in at least two different reference conformations, including at least one ligand-bound conformation, and (c) based on said screening, providing a panel of first library members, said panel comprising members which differ with respect to their ability to binding to the receptor, depending on its conformation. However, as noted above, at least one panel member is obtained according to the major preferred screening embodiment. More preferably, a plurality, all, or substantially all are so obrtained.

The panel then preferably comprises at least two of (i)-(iii) below:

(i) at least one member which binds the ligand-bound receptor more strongly than it binds the unliganded receptor, and which detectably binds the unliganded receptor,

(ii) at least one member which binds the ligand-bound receptor less strongly than it binds the unliganded receptor, and

(iii) at least one member which binds the ligand-bound receptor about as strongly as it binds the unliganded receptor, and detectably binds both.

The present invention also includes all of the peptides set forth in the tables, substantially identical peptides, and corresponding peptoids, other peptidomimetics, and other analogues, and the diagnostic, therapeutic and “fingerprinting” uses thereof.

### BRIEF DESCRIPTION OF THE DRAWINGS

FIG. 1 shows the result of a yeast two hybrid screening of a LXXLL motif-based peptide library (up to 15 a.a.) for binding to ER alpha. The ligand-dependent liquid beta-galactosidase activity of various clones is depicted as a bar chart.

FIG. 2 shows the result of a mammalian two-hybrid screening of the active peptides of FIG. 1.

FIG. 3 shows the result of a yeast two hybrid screening of an unbiased peptide library (up to 15 a.a.) for binding to androgen receptor.

FIG. 4A shows the result of a mammalian two hybrid screening of the active peptides of FIG. 3.

FIG. 4B shows the results of a fingerprint of androgen receptor ligands DHT, MPA, CYP, RU486, FLUT and DHEA with the BIOKEY® peptide panel consisting of peptides D30, 5G11, B8H3 and B8E9.

FIG. 5 shows the result of a screen for androgen receptor ligands in a collection of steroids.

FIG. 6A shows the restriction map and multiple cloning site of pVP16. FIG. 6B shows the same information for pM.

### DETAILED DESCRIPTION OF THE PREFERRED EMBODIMENTS OF THE INVENTION

Receptors Generally

Many pharmacologically active substances elicit a specific physiological response by interacting with an element, known as a receptor, of the target cell. A receptor is a component, usually macromolecular, of an organism with which a chemical agent interacts in some specific fashion to cause an action which leads to an observable biological effect. The term is also applied to non-naturally occurring polypeptides which comprise a domain substantially identical in amino acid sequence to such a component, or a domain thereof, and which are able, when expressed in a cell to mediate a biological response by that cell to some chemical agent. For purposes of the present invention, antibodies are not considered receptors.

The term “receptor” includes both surface and intracellular receptors. Nuclear receptors are of particular interest.

An important class of receptors are proteins embedded in the phospholipid bilayer of cell membranes. The binding of an agonist to the receptor (typically at an extracellular binding site) can cause an allosteric change at an intracellular site, altering the receptor's interaction with other biomolecules. The physiological response is initiated by the interaction with this “second messenger” (the agonist is the “first messenger”) or “effector” molecule.

Enzymes are special types of receptors. Receptors interact with agonists to form complexes which elicit a biological response. Ordinary receptors then release the agonist intact. With enzymes, the agonists are enzyme substrates, and the enzymes catalyze a chemical modification of the substrate. Thus, enzyme substrates are “ligands”. Enzymes are not necessarily membrane-bound proteins; they may be intracellular proteins. Often, enzymes are activated by the action of a receptor's second messenger, or, more indirectly, by the product of an “upstream” enzymatic reaction.

Not all enzymes are receptors. Extracellular enzymes, e.g., serum enzymes, are not receptors because they do not transduce a signal into a target cell. However, they are of interest as possible target molecules in the broader embodiments of the invention.

Receptors may be monomeric or oligomeric, and, in the latter case, may be homooligomeric or heterooligomeric.

One class of receptor are protein kinases; plasma membrane-bound proteins that act by phosphorylating target proteins. Some phosphorylate tyrosine residues and others phosphorylate serine or threonine residues. These proteins typically comprise an extracellular, ligand-binding domain, and an intracellular catalytic (kinase) domain.

A related family of receptors lack the intracellular kinase domain but, in response to agonist, activate independent membrane-embedded or cytosolic protein kinases.

Another class of membrane receptors comprise an extracellular ligand binding domain and an intracellular domain which is a guanylyl cyclase, synthesizing the second messenger cyclic AMP.

Some receptors form ion-selective channels in the plasma membrane, conveying a signal by altering the cell's membrane potential or ionic composition. These include the nictotinic choloinergic receptor, the GABA_{A} receptor, and receptors for Glu, Asp and Gly.

G protein coupled receptors are hydrophobic proteins that span the plasma membrane in seven alpha helical segments. Ligands may bind in a pocket formed by these helices, or to a separate extracellular domain. The receptors interact with one or more G proteins at their cytoplasmic face.

The term “soluble receptors” literally refers to any receptor which is not bound. Hence, it includes any intracellular receptors found free in the cytoplasm. However, the term is also applied to a fragment corresponding to the extracellular ligand binding domain, of a membrane receptor. These fragments are not really receptors at all, but rather antagonists for the original membrane receptor (which they compete with for ligand). Such fragments are still potential “target molecules” even though they are not “target receptors”.

The ligand-binding fragment may also be conjugated to a signal transducing domain to form a chimeric receptor. Moreover, artificial receptors may be formed by conjugating a ligand-binding target molecule to a signal transducing domain (forming an artificial receptor) in such manner that ligand binding, in a suitable cellular environment, results in signal transduction.

Receptors are discussed in more detail in the “Target Receptor” section, infra.

Receptor-Mediated Pharmacological Activity

Hormones, growth factors, neurotransmitters and many other biomolecules normally act through interaction with specific cellular receptors. Drugs may activate or block particular receptors to achieve a desired pharmaceutical effect. Cell surface receptors mediate the transduction of an “external” signal (the binding of a ligand to the receptor) into an “internal” signal (the modulation of a pathway in the cytoplasm or nucleus involved in the growth, metabolism or apotosis of the cell).

In many cases, transduction is accomplished by the following signaling cascade:

An agonist (the ligand) binds to a specific protein (the receptor) on the cell surface.

As a result of the ligand binding, the receptor undergoes an allosteric change which activates a transducing protein in the cell membrane.

The transducing protein activates, within the cell, production of so-called “second messenger molecules.”

The second messenger molecules activate certain regulatory proteins within the cell that have the potential to “switch on” or “off” specific genes or alter some metabolic process.

This series of events is coupled in a specific fashion for each possible cellular response. The response to a specific ligand may depend upon which receptor a cell expresses. For instance, the response to adrenalin in cells expressing α-adrenergic receptors may be the opposite of the response in cells expressing β-adrenergic receptors.

The above “cascade” is idealized, and variations on this theme occur. For example, a receptor may act as its own transducing protein, or a transducing protein may act directly on an intracellular target without mediation by a “second messenger”.

The substances which are able to elicit the response, by specific interaction with a receptor site, are known as agonists. Typically, increasing the concentration of the agonist at the receptor site leads to an increasingly larger response, until a maximum response is achieved. A substance able to elicit the maximum response is known as a full agonist, and one which elicits only, at most, a lesser (but discernible) response is a partial agonist.

A pharmacological antagonist is a compound which interacts with the receptor without eliciting a response, and by doing so inhibits the receptor from responding to agonists. A competitive antagonist is one whose effect can be overcome by increasing the agonist concentration; a noncompetitive antagonist is one whose action is unaffected by agonist concentration. A sequestering antagonist is one which inhibits a ligand: receptor interaction by binding to the ligand in such a way that it can no longer bind the receptor. A competitive sequestering antagonist competes with the receptor for the ligand, whereas a competitive pharmacological antagonist competes with the ligand for the receptor.

Ligands are substances which bind to receptors, and thereby encompass both agonists and pharmacological antagonists. However, ligands exist which bind receptors, but which neither agonize nor antagonize the receptor. Ligands which activate (agonize) or inhibit (antagonize) the receptor are here collectively termed modulators. Some modulators change roles, acting as agonists or antagonists, depending on circumstances.

Natural ligands are those which, in nature, without human intervention, are responsible for agonizing or antagonizing a natural receptor. A natural ligand may be produced by the organism to which the receptor is native. A ligand native to a pathogen or parasite may bind to a receptor native to a host. Or a ligand native to a host may bind to a receptor native to a pathogen or parasite. All of these are natural ligands.

The clinical concept of drug antagonism is broader than the pharmacological concept, including phenomena that do not involve direct inhibition of agonist:receptor binding. A “physiological” antagonist could be a substance which directly or indirectly inhibits the production, release or transport to the receptor site of the natural agonist, or directly or indirectly facilitates its elimination (whether physical, or by modification to an inactive form) from the receptor site, or inhibits the production or increases the rate of turnover of the receptor, or interferes with signal transduction from the activated receptor.

A physiological antagonist of one receptor (e.g., an estrogen receptor) may be a pharmacological antagonist of another, e.g., a transcription factor. A physiological antagonist of one receptor may be a pharmacological agonist of another receptor, such as one which activates an enzyme which degrades the natural ligand of the first receptor.

Similarly, one may speak of a physiological agonist, which is a substance which directly or indirectly enhances the production, release or transport to the receptor site of the natural agonist, or directly or indirectly inhibits its elimination from the receptor site, or enhances the production or reduces the rate of turnover of the receptor, or in some way facilitates signal transduction from the activated receptor.

It follows that there are both “pharmacological” and “physiological” modulators.

A functional antagonist of a receptor is a substance which acts on a second receptor triggering a biological response which counteracts or inhibits the normal response to activation of the first receptor. Thus, a functional antagonist of one receptor may be a pharmacological agonist of another.

If a disease state is the result of inappropriate activation of a receptor, the disease may be prevented or treated by means of a physiological or pharmacological antagonist. Other disease states may arise through inadequate activation of a receptor, in which case the disease may be prevented by means of a suitable physiological or pharmacological agonist.

Since enzymes are receptors, drugs may also be useful because of their interaction with enzymes. The drug may serve as a substrate for the enzyme, as a coenzyme, or as an enzyme inhibitor. (An irreversible inhibitor is an “inactivator”.) Drugs may also cause, directly or indirectly, the conversion of a proenzyme or apoenzyme into an enzyme. Many disease states are associated with inappropriately low or high activity of particular enzymes.

Both agonists and co-activators bind to a receptor, and increase its level of activation (signal transduction; enzymatic activity; etc.). However, an agonist binds to a ligand binding site which is exposed even in the absence of a co-activator. A co-activator binds a receptor only after an agonist binds, the receptor, causing a change in conformation which opens up the co-activator's binding site. Agonist binding is coactivator-independent, although the coactivator may be necessary to activate the receptor. A co-activator may be facultative or obligatory. A co-inhibitor competitively inhibits the binding of a co-activator to the co-activator binding site.

The present invention may be used to identify agonists, antagonists, and coactivators and coinhibitors, of receptors. It is not unusual for a relatively small structural change to convert an agonist into a pharmacological antagonist, or vice versa. Therefore, even if the drugs known to interact with a reference protein are all agonists, the drugs in question may serve as leads to the identification of both agonists and antagonists of the reference protein and of related proteins. Similarly, known antagonists may serve as drug leads, not only to additional antagonists, but to agonists as well.

Cell-Based Screening Assays of Combinatorial Libraries

In a cell-based screening of a combinatorial peptide library for peptide binding to unliganded receptor, each cell coexpresses the receptor and one peptide of the library, and a signal producing system for differentiating binding and nonbinding peptides is provided.

If the receptor is a surface receptor, the peptide must be secreted, and the signal producing system must be stimulated by the binding of the secreted peptide to the receptor. If the receptor is intracellular, the peptide and receptor must be coexpressed in such manner that they encounter each other. Nuclear receptors are of particular interest.

To carry out a cell-based assay for the binding of a peptide to a particular liganded receptor conformation, the ligand must have access to the receptor. Such access may be provided by incubating the cell with the ligand (or a precursor of the ligand which the cell processes to produce the ligand) so that it can access the receptor, or by engineering the cell to produce the receptor. In the latter case, the ligand, if a peptide, may be produced directly. If the ligand is not a peptide, the cell may be engineered so as to enzymatically produce the ligand from intracellular starting materials. Preferably, the ligand is exogenously provided.

The same choices (in vitro vs cell-based assay) exist for screening reference and test compounds, too. Thus, it is possible to use a cell-based assay to identify, in a library, peptides which bind the receptor in one conformation (e.g., unliganded), and subsequently determine the sensitivity of this binding to liganded receptor conformation by in vitro assays. Or vice versa. Or BioKeys may be identified by an in vitro assay and reference and test compounds by cell-based assays. Or vice versa.

It should further be noted that, in another but related aspect, the invention relates to a cell-based assay (in particular a “two-hybrid” assay) for screening a combinatorial peptide library for binding to a receptor in the presence of an exogenously added ligand (e.g., estrogen receptor in the presence of estradiol). In a preferred embodiment, these aspects are combined, that is, each cell co-expresses the receptor and one member of the peptide library, and one or more ligands (agonists and/or antagonists) are exogenously provided.

In yet another aspect, the invention relates to a cell-based assay (in particular a “two-hybrid” assay) for screening a combinatorial peptide library for binding to a nuclear receptor, in particular the estrogen, androgen, and glucocorticoid receptors.

If a peptide is found which binds a receptor, co-expressed or not, it may be used for any therapeutic or diagnostic purpose for which a receptor-binding molecule is suited, and such uses are within the contemplation of the invention. However, for a peptide to be useful as a Biokey, it must be conformation-specific, that is, it must differ substantially in its affinity for the receptor depending on its conformation, e.g., bind the receptor in the presence of ligand A but not of ligand B, or in the presence of ligand but not in the absence of ligand.

One may also use this system to screen for binding to a target protein that is not a receptor. The advantage is that the target would not have to be made as a purified protein or greatly overexpressed to identify binding partners. Low level of expression from an expression plasmid is enough to generate a specific signal.

In Vitro vs. In Vivo Assays; Cell-Based vs. Orgasmic Assays

The term “in vivo” is descriptive of an event, such as binding or enzymatic action, which occurs within a living organism. The organism in question may, however, be genetically modified. The term “in vitro” refers to an event which occurs outside a living organism. Parts of an organism (e.g., a membrane, or an isolated biochemical) are used, together with artificial substrates and/or conditions. For the purpose of the present invention, the term in vitro excludes events occurring inside or on an intact cell, whether of a unicellular or multicellular organism.

In vivo assays include both cell-based assays, and organismic assays. The term cell-based assays includes both assays on unicellular organisms, and assays on isolated cells or cell cultures derived from multicellular organisms. The cell cultures may be mixed, provided that they are not organized into tissues or organs. The term organismic assay refers to assays on whole multicellular organisms, and assays on isolated organs or tissues of such organisms.

“Biological assays” include both in vivo assays, and in vitro assays on subcellular multimolecular components of cells such as membranes.

Cell-Based Assays

In a preferred cell-based assay, the receptor is functionally connected to a signal (biological marker) producing system, which may be endogenous or exogenous to the cell.

“Zero-Hybrid” Systems

In these systems, the binding of a peptide to the target protein results in a screenable or selectable phenotypic change, without resort to fusing the target receptor (or a ligand binding moiety thereof) to an endogenous protein. It may be that the target protein is endogenous to the host cell, or is substantially identical to an endogenous receptor so that it can take advantage of the latter's native signal transduction pathway. Or sufficient elements of the signal transduction pathway normally associated with the target protein may be engineered into the cell so that the cell signals binding to the target protein.

“One-Hybrid” Systems

In these systems, a chimeric receptor, a hybrid of the target receptor and an endogenous receptor, is used. The chimeric receptor has the ligand binding characteristics of the target protein and the signal transduction characteristics of the endogenous receptor. Thus, the normal signal transduction pathway of the endogenous receptor is subverted.

Preferably, the endogenous receptor is inactivated, or the conditions of the assay avoid activation of the endogenous receptor, to improve the signal-to-noise ratio.

See Fowlkes U.S. Pat. No. 5,789,184 for a yeast system.

Another type of “one-hybrid” system combines a peptide: DNA-binding domain fusion with an unfused target receptor that possesses an activation domain.

“Two-Hybrid” System

In a preferred embodiment, the cell-based assay is a two hybrid system. This term implies that the ligand is incorporated into a first hybrid protein, and the receptor into a second hybrid protein (a chimeric receptor). The first hybrid also comprises component A of a signal generating system, and the second hybrid comprises component B of that system. Components A and B, by themselves, are insufficient to generate a signal. However, if the ligand binds the receptor, components A and B are brought into sufficiently close proximity so that they can cooperate to generate a signal.

Components A and B may naturally occur, or be substantially identical to moieties which naturally occur, as components of a single naturally occurring biomolecule, or they may naturally occur, or be substantially identical to moieties which naturally occur, as separate naturally occurring biomolecules which interact in nature.

Two-Hybrid System: Transcription Factor Type

In a preferred “two-hybrid” embodiment, one member of a peptide ligand:receptor binding pair is expressed as a fusion to a DNA-binding domain (DBD) from a transcription factor (this fusion protein is called the “bait”), and the other is expressed as a fusion to a transactivation domain (TAD) (this fusion protein is called the “fish”, the “prey”, or the “catch”). The transactivation domain should be complementary to the DNA-binding domain, i.e., it should interact with the latter so as to activate transcription of a specially designed reporter gene that carries a binding site for the DNA-binding domain. Naturally, the two fusion proteins must likewise be complementary.

This complementarity may be achieved by use of the complementary and separable DNA-binding and transcriptional activator domains of a single transcriptional activator protein, or one may use complementary domains derived from different proteins. The domains may be identical to the native domains, or mutants thereof. The assay members may be fused directly to the DBD or TAD, or fused through an intermediated linker.

The target DNA operator may be the native operator sequence, or a mutant operator. Mutations in the operator may be coordinated with mutations in the DBD and the TAD. An example of a suitable transcription activation system is one comprising the DNA-binding domain from the bacterial repressor LexA (PEG202 vector for LexA DBD is sequence deposit U89960) and the activation domain from the yeast transcription factor Gal4, with the reporter gene operably linked to the LexA operator (Access J01643). Or one could use the yeast Gal4 DNA BD and yeast Gal4 operator(Gall Access K02115, Gal2 Access M81879, Gal7 Access M12348).

It is not necessary to employ the intact target receptor; just the ligand-binding moiety is sufficient.

The two fusion proteins may be expressed from the same or different vectors. Likewise, the activatable reporter gene may be expressed from the same vector as either fusion protein (or both proteins), or from a third vector.

Potential DNA-binding domains include Gal4, LexA, and mutant domains substantially identical to the above.

Potential activation domains include E. coli B42 (PJG4-5 vector Access 489961), Gal4 activation domain II, and HSV VP16 (Access M57289), and mutant domains substantially identical to the above.

Patents relating to Gal4, VP1.6, or mutants thereof include JP607876A2, U.S. Pat. No. 6,087,166, and EP743520.

Potential operators include the native operators for the desired activation domain, and mutant operators substantially identical to the native operator.

The fusion proteins may comprise nuclear localization signals, such as SV40 large T antigen NLS (Access P03070).

The assay system will include a signal producing system, too. The first element of this system is a reporter gene operably linked to an operator responsive to the DBD and TAD of choice. The expression of this reporter gene will result, directly or indirectly, in a selectable or screenable phenotype (the signal). The signal producing system may include, besides the reporter gene, additional genetic or biochemical elements which cooperate in the production of the signal. Such an element could be, for example, a selective agent in the cell growth medium. There may be more than one signal producing system, and the system may include more than one reporter gene.

The sensitivity of the system may be adjusted by, e.g., use of competitive inhibitors of any step in the activation or signal production process, increasing or decreasing the number of operators, using a stronger or weaker DBD or TAD, etc.

When the signal is the death or survival of the cell in question, or proliferation or nonproliferation of the cell in question, the assay is said to be a selection. When the signal merely results in a detectable phenotype by which the signalling cell may be differentiated from the same cell in a nonsignalling state (either way being a living cell), the assay is a screen. However, the term “screening assay” may be used in a broader sense to include a selection. When the narrower sense is intended, we will use the term “nonselective screen”.

Various screening and selection systems are discussed in Ladner, U.S. Pat. No. 5,198,346.

Screening and selection may be for or against the peptide: target protein or compound:target protein interaction.

Preferred assay cells are microbial (bacterial, yeast, algal, protozooal), invertebrate (esp. mammalian, particularly human). The best developed two-hybrid assays are yeast and mammalian systems.

Normally, two hybrid assays are used to determined whether a protein X and a protein Y interact, by virtue of their ability to reconstitute the interaction of the DBD and the TAD. However, augmented two-hybrid assays have been used to detect interactions that depend on a third, non-protein ligand.

For more guidance on two-hybrid assays, see Brent and Finley, Jr., Ann. Rev. Genet., 31:663-704 (1997); Fremont-Racine, et al., Nature Genetics, 277-281 (16 July 1997); Allen, et al., TIBS, 511-16 (December 1995); LeCrenier, et al., BioEssays, 20:1-6 (1998); Xu, et al., Proc. Nat. Acad. sci. (USA), 94:12473-8 (November 1992); Esotak, et al., Mol. Cell. Biol., 15:5820-9 (1995); Yang, et al., Nucleic Acids Res., 23:1152-6 (1995); Bendixen, et al., Nucleic Acids Res., 22:1778-9 (1994); Fuller, et al., BioTechniques, 25:85-92 (July 1998); Cohen, et al., PNAS (USA) 95:14272-7 (1998); Kolonin and Finley, Jr., PNAS (USA) 95:14266-71 (1998). See also Vasavada, et al., PNAS (USA), 88:10686-90 (1991) (contingent replication assay), and Rehrauer, et al., J. Biol. Chem., 271:23865-73 91996) (LexA repressor cleavage assay).

Two-Hybrid Systems: Reporter Enzyme type

In another embodiment, the components A and B reconstitute an enzyme which is not a transcription factor. It may, for example, be DHFR, or one of the other enzymes identified in WO98/34120. As in the last example, the effect of the reconstitution of the enzyme is a phenotypic change which may be a screenable change, a selectable change, or both.

Universite de Montreal, WO98/34120 describes the use of protein-fragment complementation assays to detect biomolecular interactions in vivo and in vitro. Fusion peptides respectively comprising N and C terminal fragments of murine DHFR were fused to GCN4 leucine zipper sequences and co-expressed in bacterial cells whose endogenous DHFR activity was inhibited. DHFR is composed of three structural fragments forming two domains; the discontinuous 1-46 and 106-186 fragments form one domain and the 47-105 fragment forms the other. WO98/34120 cleaved DHFR at residue 107. GCN4 is a homodimerizing protein. The homodimerization of GCN4 causes reassociation of the two DHFR domains and hence reconstitution of DHFR activity.

WO98/34120 suggest that fragments of other enzyme reporter molecules could be used in place of DHFR.

See also, Pelletier, et al., Proc. Nat. Acad. Sci. USA, 95: 12141-6 (1998)(same system);

Karimova et al., Proc. Nat. Acad. Sci. USA 95:5752-6 (1998) discloses a bacterial two-hybrid system, in which the catalytic domain of Bordetella pertussis adenylate cyclase reconstituted as a result of interaction of two proteins, leading to cAMP synthesis).

In a similar system, designed to distinguish heterodimerization as distinct from homodimerization, one test protein was fused to native LexA and the other to a mutant of LexA with altered DNA specificity. Normally, LexA dimerizes to bind its target operator. Because of the mutation, and the use of a hybrid operator, only a heterodimer could achieve DNA binding. See Dmitrova, et al., Mol. Gen. Genet., 57: 205-212 (1998).

Stanford U., WO98/44350 describes a reporter subunit complementation assay which employs fusion proteins each compromising one of a pair of weakly complementing, singly inactive, beta galactosidase mutants, which complement each other to produce an active beta galactosidase. See also Rossi, et al., Proc. Nat. Acad. Sci. USA, 94:8405-10 (1997); Mohler and Blau, Proc. Nat. Acad. Sci. USA, 93: 12423-7 (1996).

Cornell U., WO98/34948 describes a strategy for the identification of small peptides that activate or inactivate a G protein coupled receptor. The peptides of a combinatorial peptide library are tethered to a GPCR of interest in a cell, and the cell is monitored to determine whether the peptide is an agonist or an antagonist. The peptide is tethered to the GPCR by replacing the N-terminal of the GPCR with the N-terminus of a self-activating receptor, and replacing the natural peptide ligand present therein with the library peptide. An example of a self-activating receptor would be the thrombin receptor.

Sadee, U.S. Pat. No. 5,882,944 discloses a cell-based assay for the effect of test compounds on ml receptors in which the cells are incubated with an ml agonist to constitutively activate them, the agonist is removed, the baseline activity of the receptor is determined, the cells are exposed to the test compound, and the receptor activity is compared to the baseline level. The activity measured may be directed to cAMP, GTPase, or GTP exchange.

Martin, et al., J. Biol. Chem., 271: 361-6 (1996) describes the screening of a combinatorial peptide-on-plasmid library based on the C terminus of the alpha subunit of Gsubt (340-350) for peptides which bind rhodopsin. In the library, the library peptides are fused to the C terminus of the DNA binding protein lacI, which binds to lacO DNA sequences on the vector expressing the peptide. In the random DNA, the base mix was chosen so as to yield roughly a 50% chance that a given codon would be mutated to yield a different amino acid.

Stables, et al., Anal. Biochem., 252: 115-126 (1997) describes a cell-based bioluminescent assay for GPCR agonist activity. The GPCR is co-expressed with apoaequorin, a calcium-sensitive photoprotein. Agonist binding to a receptor which activates certain G-alpha subunits, such as G-alpha16, results in an increase in intracellular calcium concentration and subsequent bioluminescence.

Cells for Screening

The intracellular screening assay is carried out in cells which functionally express a suitable target receptor. If the assay is a two-hybrid assay, the target will be a chimeric receptor, and the cells will have been genetically engineered to express it. The cells will in any even by genetically engineered to each express one (preferably only one) member of the peptide library.

Preferably, the cells are eukaryotic cells. The cells may be from a unicellular organism, a multicellular organism (including a colonial organism) or an intermediate from (slime mold). If from a multicellular organism, the latter may be an invertebrate, a lower vertebrate (reptile, fish, amphibian) or a higher vertebrate (bird, mammal). The organism may be aquatic (fresh or saltwater), or terrestrial, or both, in habitat. More preferably, the cells are non-mammalian eukaryotic cells.

In one embodiment the cells are yeast cells. Preferably, the yeast cells are of one of the following genera: Saccharomyces, Schizosaccharomyces, Candida, Hansenula, Pichia, Kluyveromyces, Cryptococcus, Yarrowia and Zygosaccharomyces.

More preferably, they are of one of the following species:

Saccharomyces cerevisiae (budding, baker's and sometimes brewer's)

Saccharomyces bayanus

Saccharomyces boulardii

Saccharomyces carlsbergensis

Saccharomyces chevalieri

Saccharomyces chodati

Saccharomyces diastaticus

Schizosaccharomyces pombe (fission)

Candida albicans

Candida boidnii (source of peroxisomes)

Candida tropicalis

Candida sake

Hansenula polymorpha (source of peroxisomes)

Pichia pastoris (source of peroxisomes)

Kluyveromyces lactis

Cryptococcus neoformans

Cryptococcus laurentii, C. uniguttulatus, C. hungaricus, C. magnus, C. albidus, C. alter, C. curvatus, C. dimennae, C. humicolus and C. infirmominiatus (maybe more relevant to food industry)

Yarrowia lipolytica

Zygosaccharomyces rouxii

Other non-mammalian cells of interest include plant cells (e.g., Arabidopsis) arthropod (incl. insect) cells, annelid or nematode cells (e.g., Caenorhabditis elegans; planaria; leeches; earthworms; polychaetus annelids), crustaceans (e.g., daphnia), protozoal cells (e.g. Dictyostelium discoideum), and lower vertebrate (reptiles, amphibians, fish) cells. For fish, the preferred cells are from trout, salmon, carps, tilapia, medaka, goldfish, zebrafish, loach and catfish. For amphibians, the preferred cells are from Xenopus and Rana.

Among marine invertebrates, cells of interest include those from Aplysia (sea slug); corals, jellyfish and sea anemones; crustaceous (e.g., daphnids); squids and octopi, and horseshoe crabs.

Fleer, R. (1992) Curr. Opin. In Biotech. 3(5):486-496 reviews use of non-mammalian eukaryotic cells, such as insect cells, Sp. frugiperda, and yeast (s. cerevisiae, S. pombe, P. pastoris, K. lactis, and H. polymorpha) for transactivation studies.

While mammalian cells are not preferred, because of their greater difficulty of cell culture, they may be used. Preferably, they are used for confirmatory analysis of peptides already preliminarily identified as active, rather than in screening a peptide library in the first instance.

Combinatorial Libraries

The term “library” generally refers to a collection of chemical or biological entities which are related in origin, structure, and/or function, and which can be screened simultaneously for a property of interest.

The term “combinatorial library” refers to a library in which the individual members are either systematic or random combinations of a limited set of basic elements, the properties of each member being dependent on the choice and location of the elements incorporated into it. Typically, the members of the library are at least capable of being screened simultaneously. Randomization may be complete or partial; some positions may be randomized and others predetermined, and at random positions, the choices may be limited in a predetermined manner. The members of a combinatorial library may be oligomers or polymers of some kind, in which the variation occurs through the choice of monomeric building block at one or more positions of the oligomer or polymer, and possibly in terms of the connecting linkage, or the length of the oligomer or polymer, too. Or the members may be nonoligomeric molecules with a standard core structure, like the 1,4-benzodiazepine structure, with the variation being introduced by the choice of substituents at particular variable sites on the core structure. Or the members may be nonoligomeric molecules assembled like a jigsaw puzzle, but wherein each piece has both one or more variable moieties (contributing to library diversity) and one or more constant moieties (providing the functionalities for coupling the piece in question to other pieces).

The ability of one or more members of such a library to recognize a target molecule is termed “Combinatorial Recognition”. In a “simple combinatorial library”, all of the members belong to the same class of compounds (e.g., peptides) and can be synthesized simultaneously. A “composite combinatorial library” is a mixture of two or more simple libraries, e.g., DNAs and peptides, or benzodiazepine and carbamates. The number of component simple libraries in a composite library will, of course, normally be smaller than the average number of members in each simple library, as otherwise the advantage of a library over individual synthesis is small.

Preferably, a combinatorial library will have a diversity of at least 100, more preferably at least 1,000, still more preferably at least 10,000, even more preferably at least 100,000, most preferably at least 1,000,000, different molecules.

Usually, the diversity of the combinatorial library will be less than 10¹⁶, more usually not more than 10¹³, still more usually not more than 10¹⁰, most usually in the range of 10⁴ to 10⁹.

In the case of oligomeric combinatorial libraries, at each variable oligomeric position, the number of choices of monomer will usually be in the range of 2-100, more often 2-50, most often 2-20 for peptide libraries and 2-4 for nucleic acid libraries. The number and nature of choices may vary from position to position. The number of variable sites will preferably be in the range of 1-30, more preferably 2-20, most preferably 4-15.

The overall diversity is the product, over all variable positions, of the number of choices at that position. If the number of choices is the same for each position, it is the number of choices raised to the power of the number of variable positions.

If the combinatorial library is to be expressed, it will be a peptide-library as described below.

Peptide Library

A peptide library is a combinatorial library, at least some of whose members are peptides having three or more amino acids connected via peptide bonds. Preferably, they are at least five, six, seven or eight amino acids in length. Preferably, they are composed of less than 50, more preferably less than 20 amino acids.

The peptides may be linear, branched, or cyclic, and may include nonpeptidyl moieties. The amino acids are not limited to the naturally occurring amino acids. Preferably, the individual amino acids are not larger than 1000 daltons.

A biased peptide library is one in which one or more (but not all) residues of the peptides are constant residues. The individual members are referred to as peptide ligands (PL). In one embodiment, an internal residue is constant, so that the peptide sequence may be written as

(Xₐₐ)ₘ-AA₁-(Xₐₐ)ₙ

Where Xaa is either any naturally occurring amino acid, or any amino acid except cysteine, m and n are chosen independently from the range of 2 to 20, the Xaa may be the same or different, and AA₁ is the same naturally occurring amino acid for all peptides in the library but may be any amino acid. Preferably, m and n are chosen independently from the range of 4 to 9.

Preferably, AA₁ is located at or near the center of the peptide. More specifically, it is desirable that m and n are not different by more than 2; more preferably m and n are equal. Even if the chosen AA₁ is required (or at least permissive) of the target protein (TP) binding activity, one may need particular flanking residues to assure that it is properly positioned. If AA₁ is more or less centrally located, the library presents numerous alternative choices for the flanking residues. If AA₁ is at an end, this flexibility is diminished.

The most preferred libraries are those in which AA₁ is tryptophan, proline or tyrosine. Second most preferred are those in which AA₁ is phenylalanine, histidine, arginine, aspartate, leucine or isoleucine. Third most preferred are those in which AA₁ is asparagine, serine, alanine or methionine. The least preferred choices are cysteine and glycine. These preferences are based on evaluation of the results of screening random peptide libraries for binding to many different TPs.

Ligands that bind to functional domains tend to have both constant as well as unique features. Therefore, by using “biased” peptide libraries, one can ease the burden of finding ligands. Either “biased” or “unbiased” libraries may be screened to identify “BioKey” peptides for use in developing reactivity descriptors, and, optionally, peptide aptamer descriptors and additional drug leads.

Target Receptor

The target receptor may be a naturally occurring substance, or a subunit or domain thereof, from any natural source, including a virus, a microorganism (including bacterial, fungi, algae, and protozoa), an invertebrate (including insects and worms), or the normal or cancerous cells of a vertebrate (especially a mammal, bird or fish and, among mammals, particularly humans, apes, monkeys, cows, pigs, goats, llamas, sheep, rats, mice, rabbits, guinea pigs, cats and dogs). (Usually it is a protein; it may be a nucleic acid. References to proteins apply, mutatis mutandis, to nucleic acids, lipids, carbohydrates and other macromolecules which can act as receptors.) Alternatively, the receptor protein may be a modified form of a natural receptor. Modifications may be introduced to facilitate the labeling or immobilization of the target receptor, or to alter its biological activity (An inhibitor of a mutant receptor may be useful to selectively inhibit an undesired activity of the mutant receptor and leave other activities substantially intact). In the case of a protein, modifications include mutation (substitution, insertion or deletion of a genetically encoded amino acid) and derivatization (including glycosylation, phosphorylation, and lipidation). The target may a chimera of two receptors, e.g., a mammalian and a yeast receptor, or two receptors of different functions, so as to combine the ligand binding function of one receptor with the signal transduction function of another.

A target receptor may be, inter alia, a glyco-, lipo-, phosphor or metalloprotein. It may be a nuclear, cytoplasmic, membrane, or secreted protein. It may, but need not, be an enzyme.

The target receptor, instead of being a protein, may be a macromolecular nucleic acid, lipid or carbohydrate. If a nucleic acid, it may be a ribo- or a deoxyribonucleic acid, and it may be single or double stranded. It may, but need not, have enzymatic activity.

The target receptor need not be a single macromolecule, rather, it may be a complex of a macromolecule with one or more additional molecules, especially macromolecules. Examples includes ribosomes (RNA:protein complexes), polysomes (mRNA:ribosome complexes), and chromatin (DNA:protein complexes). For use of polysomes as binding molecules (or as display systems), see Kawasaki, U.S. Pat. No. 5,643,768 and 5,658,754; Gersuk, et al., Biochem. Biophys. Res. Comm. 232:578 (1997); Mattheakis, et al., Proc. Nat. Acad. Sci. USA, 91:9022-6 (1994).

The known binding partners (if any) of the target receptor may be, inter alia, proteins, oligo- or polypeptides, nucleic acids, carbohydrates, lipids, or small organic or inorganic molecules or ions.

The functional groups of the receptor which participate in the ligand-binding interactions together form the ligand binding site, or paratope, of the receptor. Similarly, the functional groups of the ligand which participate in these interactions together form the epitope of the ligand.

In the case of a protein, the binding sites are typically relatively small surface patches. The binding characteristics of the protein may often be altered by local modifications at these sites, without denaturing the protein.

While it is possible for a chemical reaction to occur between a functional group on a receptor and one on a ligand, resulting in a covalent bond, receptor protein-ligand binding normally occurs as a result of the aggregate effects of several noncovalent interactions. Electrostatic interactions include salt bridges, hydrogen bonds, and van der Waals forces.

What is called the hydrophobic interaction is actually the absence of hydrogen bonding between nonpolar groups and water, rather than a favorable interaction between the nonpolar groups themselves. Hydrophobic interactions are important in stabilizing the conformation of a receptor protein and thus indirectly affect ligand binding, although hydrophobic residues are usually buried and thus not part of the binding site.

The receptor may have more than one paratope and they may be the same or different. Different paratopes may interact with epitopes of different binding partners. An individual paratope may be specific to a particular binding partner, or it may interact with several different binding partners. A receptor can bind a particular binding partner through several different binding sites. The binding sites may be continuous or discontinuous (e.g., vis-a-vis the primary sequence of a receptor protein).

A list of agonists, antagonists, radioligands and effectors for many different receptors appears in Appendix I of King, Medicinal Chemistry: Principles and Practice, pp. 290-294 (Royal Soc'y Chem. 0.1994). Appendix II lists blockers for various ion channels (which are another special type of receptor). Some receptors, and their agonists and/or antagonists, are listed in Table A.

Any nuclear receptor, such as receptors for progestins, androgens, glucocorticoids, thyroid hormones, retinoids, vitamin D3 and mineralocorticoids could be used in this fingerprinting system. Affinity selection of peptide libraries could be used to identify peptide sequences that bind in the presence or absence of agonist as described above. The peptides could then be used in the manner described above to classify and characterize modulators of the receptor's activity. As described above, components of Premarin are likely to interact with the progesterone receptor. A system for fingerprinting the progesterone receptor may be developed to test for active components of Premarin.

As an example of a non-protein receptor, we cite DNA. DNA can undergo conformational changes when it is bound for example, by a transcription factor or small molecule. For example, the antitumor agent cisplatin binds to and alters the structure of DNA. The altered structure attracts a cellular protein containing an HMG box (high mobility group). The protein is believed to sterically block the repair of the cisplatin lesion on the DNA and contribute to the effectiveness of cisplatin in the treatment of certain types of cancer. BioKeys could be identified that bind specifically to DNA in certain conformations. These Biokeys could be used to identify conformational changes that take place in the DNA upon binding of a small molecule or protein.

Nuclear Receptors

Nuclear receptors are a family of ligand activated transcriptional activators, see Evans and Hollenberg, Cell, 52:1-3 (1988), factors which include the receptors for steroid and thyroid hormones, retinoids, and vitamin D. The steroid receptor family is composed of receptors for glucocorticoids, mineralocorticoids, androgens, progestins, and estrogens. These receptors are organized into distinct domains for ligand binding, dimerization, transactivation, and DNA binding. Receptor activation occurs upon ligand binding, which induces conformational changes allowing receptor dimerization and binding of co-activating proteins. These co-activators, in turn, facilitate the binding of the receptors to DNA and subsequent transcriptional activation of target genes. In addition to the recruitment of co-activating proteins, the binding of ligand is also believed to place the receptor in a conformation that either displaces or prevents the binding of proteins that serve as co-repressors of receptor function. Lavinsky, et al., Proc. Nat. Acad. Sci. (USA), 95:2920 (1998)

The estrogen receptor is a member of the steroid family of nuclear receptors. Human ERα is a 595 amino acid protein composed of six functional domains or regions (A-F). The A/B region contains the transcription function AF-1, and the E domain contains the transcription function AF-2. These functions activate transcription in a cell- and promoter context-specific manner. AF-1 is constitutively active, while AF-2 is induced by hormone binding to the receptor. The C region contains the DNA-binding domain and a dimerization domain. The DNA-binding domain binds the estrogen (receptor) response element (ERE) associated with a regulated gene. The DBD contains two zinc fingers. The C region may also be responsible for nuclear localization. The E region contains the hormone (ligand) binding domain.

The classical ERE is composed of two inverted hexanucleotide repeats, and ligand-bound ER binds to the ERE as a homodimer. The ER also mediates gene transcription from an AP1 enhancer element that requires ligand and the AP1 transcriptional factors Fos and Jun for transcriptional activation. Tamoxifen inhibits transcription of genes regulated by a classical ERE, but activates transcription of genes under the control of an AP1 element. See Paech, et al., Science, 277:1508-11 (1997).

In the absence of hormone, the estrogen receptor resides in the nucleus of target cells where it is associated with an inhibitory heat shock protein complex. (Smith, et al., (1993) Mol. Endocrinol., 7:4-11.) Upon binding ligand, the receptor is activated. This process permits the formation of stable receptor dimers and subsequent interaction with specific DNA response elements located within the regulatory region of target genes. (McDonnell, et al. (1991), Mol. Cell Biol., 11:4350-4355.) The DNA bound receptor can then either positively or negatively regulate target gene transcription. Although the precise mechanism by which the ER modulates RNA polymerase activity remains to be determined, it has been shown recently that agonist bound ER can recruit transcriptional adaptors, proteins that permit the receptor to transmit its regulatory information to the cellular transcriptional apparatus. (Onate, et al. (1995), Science, 270:1354-1357; Norris, et al. (1998), J. Biol. Chem., 273:6679-6688; Smith, et al. (1997), Mol. Endocrinol., 11:657-666). Conversely, when occupied by antagonists, the DNA bound receptor actively recruits co-repressors, proteins that permit the cell to distinguish between agonists and antagonists. (Norris, et al. (1998); Smith, et al. (1997); Lavinsky, et al., (1998) Proc. Natl. Acad. Sci. USA, 95:2920-2925). Building on this complexity was the recent discovery of a second estrogen receptor, ERβ, whose mechanism of action appears to be similar, yet distinct from ERα. (Greene, et al. (1986), Science, 231:1150-1154; Kuiper, et al. (1996), Proc. Natl. Acad. Sci. USA, 93:5925-5930; Mosselman, et al. (1996), FEBS Lett., 392:49-53).

Thus, there are two forms of this receptor, α and β, presently known; other forms may exist. Both receptors activate transcription in response to estrogens, which are an important group of steroid hormones that not only influence the growth, differentiation, and functioning of the reproductive system, but also exert effects in the bone, brain and cardiovascular system. Estrogens can produce a broad range of effects in this diverse set of target tissues. These differential effects are believed to be mediated, in part, by tissue specific activation of the two different transactivation domains present at the amino-terminal and carboxy-terminal regions of the receptor. It is also likely that the two forms of the receptor (α and β) function in distinct tissues and thereby mediate the transactivation of different subsets of genes. (Paech, et al., Science, 277:1508, 1997; Kuiper and Gustafsson, FEBS Lett., 410:87, 1997; Nichols, et al., EMBO J., 17:765, 1998; Montano, et al., Mol. Endo., 9:814, 1995.)

Drugs that target the estrogen receptor can exhibit a variety of effects in different target tissues. For example, tamoxifen is an ER antagonist in breast tissue, (Jordan, V. C., (1992) Cancer, 70:977-982), but an ER agonist in bone (Love, et al. (1992), New Engl. J. Med., 326:852-856) and uterine, (Kedar, et al. (1994), Lancet, 343:1318-1321) tissue. Raloxifene is also an ER antagonist in breast tissue; however, it exerts agonist activity in bone but not uterine tissue (Black, et al. (1994), J. Clin. Invest., 93:63-69). Indeed, one of the greatest challenges in understanding the pharmacology of the estrogen receptor is determining how different ER ligands produce such diverse biological effects.

Estrogens, in general, are stimulatory agents, resulting in increased gene expression and cell proliferation in target tissues. However, many molecules have been described that bind to the estradiol binding site on the receptor, but produce negative effects on gene expression and cell growth. These agents have historically been termed “antiestrogens”, but this term has proven to be much too simplistic. (Tremblay, et al., Can. Res., 58:877, 1988; Katzenellenboge, et al., Breast Can Res. Treatm., 44:23, 1997; Howell, Oncology (suppl. 1), 11:59, 1997; Gallo and Kaufman, Sem. in Oncol. (Suppl. 1), 24:71, 1997). One of the most noteworthy of these agents is tamoxifen, which has been successfully used in the treatment of ER-positive breast cancer. Tamoxifen, a derivative of triphenylethylene, is metabolized in the cell to produce 4-OH tamoxifen, which has very high affinity for the estradiol binding pocket of the ER. Although this compound competes with estradiol for binding to the ER, it does not induce transcriptional activation in breast tissue, thus it does not promote cell growth and acts as a classic antiestrogen in this tissue. Tamoxifen, however, does have estrogen-like activities in other tissues. In the uterus, tamoxifen acts as an agonist of receptor activity, stimulating the growth of uterine tissue leading to an increased incidence of endometrial hyperplasia in treated patients. Tamoxifen also produces estrogenic effects in the bone and cardiovascular system. This activity generates beneficial effects such as reducing the risk of osteoporosis and lowering serum LDL levels. The numerous differential effects produced by compounds such as tamoxifen has led to the replacement of the term “antiestrogen” with “selective estrogen receptor modulators” or SERMs. SERMs may have both positive and negative effects on ER activity depending on the biology of receptor and the tissue in which it is being expressed.

A goal of current research is to develop SERMs that have agonistic or estrogenic effects on bone and the cardiovascular system and antagonistic or antiestrogenic effects in the breast and uterus. One SERM that has recently been approved for treatment of post-menopausal symptoms is Raloxifene. Raloxifene is a benzothiophene derivative that, like tamoxifen, binds in the ligand binding pocket of the ER. Clinical studies indicate that this compound lacks estrogenic activity in the breast and uterus, but produces estrogenic activity in the bone and perhaps the cardiovascular system. It is currently prescribed for prevention for osteoporosis in post-menopausal women. There are several additional SERMs in clinical trials, and a great deal of effort in the pharmaceutical industry is focused on the identification and characterization of additional SERMs. The search for SERMs poses a major obstacle. In order to screen large libraries of compounds for SERMs, it is necessary to have a convenient assay for identifying which lead molecules have the desired effect(s). Currently, when a compound is identified that competes with estradiol for binding to the ER, a number of cell-based assays must be conducted to determine its activity. These studies are more laborious than in vitro assays and still do not absolutely predict the complete spectrum of biological activity of the SERM. Thus, studies often have to move into animal models or clinical trials before the selective modes of action of the SERM can be determined. A simple in vitro system to distinguish between agonist and antagonist activity of a SERM would be of great utility.

The development of such a system requires knowledge of the mechanisms that produce the broad effects of SERMs. There is evidence that SERMs are able to produce differential (agonistic and antagonistic) effects due to their ability to alter the conformation of the ER. In general, the receptor is thought of as having two conformations, active or inactive. These conformations are formed in the presence or absence of ligand, respectively. The SERM drives the receptor into a conformation that is neither fully active nor fully inactive. This intermediate conformation creates changes in the association patterns of co-activators, co-repressors, and other regulatory molecules with the receptor, thus producing variable effects. The broad range of effects produced by SERMs may also be due to selective tissue expression of ER alpha and beta as well as co-activators and co-repressors. It may also be due to different affinities of the SERM for the two receptors.

Reference Conformation

When a target receptor is in an unliganded state, it has a particular conformation, i.e., a particular 3-D structure. When the receptor is complexed to a ligand, the receptor's conformation changes. If the ligand is a pharmacological agonist, the new conformation is one which interacts with other components of a biological signal transduction pathway, e.g.; transcription factors, to elicit a biological response in the target tissue. If the ligand is a pharmacological antagonist, the new conformation is one in which the receptor cannot be activated by one or more agonists which otherwise could activate that receptor.

Each of the conformations of a target receptor which is used as a binding target in a binding array is considered a reference conformation.

It may be that two different ligands will coincidentally cause a receptor to assume the same conformation. However, for the purpose of this invention, those will be considered different reference conformations because different ligands are involved.

Reference Ligands

A reference ligand is a substance which is a ligand for a target receptor. Preferably, it is a pharmacological agonist or antagonist of a target receptor protein in one or more target tissues of a target organism. However, a reference ligand may be useful, even if it is not an agonist or antagonist, if it alters the conformation of its receptor, e.g., such that at least some Biokeys which bound the unliganded receptor do not bind as well, or bind better, the liganded receptor. Preferably, a reference ligand has a differential effect on Biokeys, so that Biokeys may be differentiated on the basis of their interaction with the receptor in the presence of the reference ligand. A reference ligand may be an agonist of one receptor and an antagonist of another. It may also be agonist of a receptor in one tissue and an antagonist of the same receptor in another tissue, or in another organism.

The reference ligand may be, but need not be, a natural ligand of the receptor.

The reference ligands may, but need not, satisfy some or all of the desiderata set forth above for test substances and drug leads.

If a test substance from one screening becomes a drug lead, and that compound, or an analogue thereof, is ultimately found to mediate the biological activity of at least one receptor in at least one tissue of at least one organism, it may be used as a reference ligand in subsequent screenings of other test substances, and in redefining the Biokey panel.

Relative Affinity

Where this specification indicates that a molecule B binds a target T1 substantially more strongly than a target T2, or that a molecule B1 binds a target T substantially more strongly than an alternative molecule B2 binds the same target T, it means that the difference in binding is detectable and is manifest to a useful degree in the relevant context, e.g., screening, diagnosis, purification, or therapy.

Generally speaking, a tenfold difference in binding will be considered substantial, however this is not necessarily required.

Potency of Antagonists

The potency of an antagonist of a receptor may be expressed as an IC50, the concentration of the antagonist which causes a 50% inhibition of a receptor's binding or biological activity in an in vitro or in vivo assay system. A pharmaceutically effective dosage of an antagonist depends on both the IC50 of the antagonist, and the effective concentrations of the receptor and its clinically significant binding partner(s).

Potencies may be categorized as follows:

| Category | IC50 |
|---|---|
| Very Weak | >1 μ moles |
| Weak | 100 n moles to 1 μ mole |
| Moderate | 10 n moles to 100 n moles |
| Strong | 1 p mole to 10 n moles |
| Very Strong | <1 p mole |

Preferably, the antagonists identified by the present invention are in one of the four higher categories identified above, and are in any event more potent than any antagonist known for the protein in question at the time of filing of this application.

In a similar manner, the potency of an agonist may be quantified as the dosage resulting in 50% of its maximal effect on a receptor.

Target Organism

A purpose of the present invention is to predict the biological activity in one or more target tissues, as hereafter defined, of a target organism.

The target organism may be a plant, animal, or microorganism. The plant or animal may be normal, chimeric or transgenic. It may or may not be infected with a pathogen (e.g., virus) or a parasite. It may be in a normal or an abnormal environmental state. It may be of a particular developmental stage, size, sex, etc.

In the case of a plant, it may be an economic plant, in which case the drug may be intended to increase the disease, weather or pest resistance, alter the growth characteristics, or otherwise improve the useful characteristics or mute undesirable characteristics of the plant. Or it may be a weed, in which case the drug may be intended to kill or otherwise inhibit the growth of the plant, or to alter its characteristics to convert it from a weed to an economic plant. The plant may be a tree, shrub, crop, grass, etc. The plant may be an algae (which are in some cases also microorganisms), or a vascular plant, especially gymnosperms (particularly conifers) and angiosperms. Angiosperms may be monocots or dicots. The plants of greatest interest are rice, wheat, corn, alfalfa, soybeans, potatoes, peanuts, tomatoes, melons, apples, pears, plums, pineapples, fir, spruce, pine, cedar, and oak.

If the target organism is a microorganism, it may be algae, bacteria, fungi, or a virus (although the biological activity of a virus must be determined in a virus-infected cell). The microorganism may be human or other animal or plant pathogen, or it may be nonpathogenic. It may be a soil or water organism, or one which normally lives inside other living things.

If the target organism is an animal, it may be a vertebrate or a nonvertebrate animal. Nonvertebrate animals are chiefly of interest when they act as pathogens or parasites, and the drugs are intended to act as a biocidic or biostatic agents. Nonvertebrate animals of interest include worms, mollusks, and arthropods.

The target organism may also be a vertebrate animal, i.e., a mammal, bird, reptile, fish or amphibian. Among mammals, the target animal preferably belongs to the order Primata (humans, apes and monkeys), Artiodactyla (e.g., cows, pigs, sheep, goats, horses), Rodenta (e.g., mice, rats) Lagomorpha (e.g., rabbits, hares), or Carnivora (e.g., cats, dogs). Among birds, the target animals are preferably of the orders Anseriformes (e.g., ducks, geese, swans) or Galliformes (e.g., quails, grouse, pheasants, turkeys and chickens). Among fish, the target animal is preferably of the order Clupeiformes (e.g., sardines, shad, anchovies, whitefish, salmon).

Target Tissues

The term “target tissue” refers to any whole animal, physiological system, whole organ, part of organ, miscellaneous tissue, cell, or cell component (e.g., the cell membrane) of a target animal in which the biological activity of a drug may be measured.

Routinely in mammals one would chose to compare and contrast the biological impact on virtually any and all tissues which express the subject receptor protein. The main tissues to use are: brain, heart, lung, kidney, liver, pancreas, skin, intestines, adrenal glands, breast, prostate, vasculature, retina, cornea, thyroid gland, parathyroid glands, thymus, bone marrow etc.

Another classification would be by cell type: B cells, T cells, macrophages, neutrophils, eosinophils, mast cells, platelets, megakaryocytes, erythrocytes, bone marrow stomal cells, fibroblasts, neurons, astrocytes, neuroglia, microglia, epithelial cells (from any organ, e.g. skin, breast, prostate, lung, intestines etc), cardiac muscle cells, smooth muscle cells, striated muscle cells, osteoblasts, osteocytes, chondroblasts, chondrocytes, keratinocytes, melanocytes, etc.

The “target tissues” include those set forth in Table B. Of course, in the case of a unicellular organism, there is no distinction between the “target organism” and the “target tissue”.

Mutant Proteins and Peptides

There are a number of instances in which the present invention contemplates the mutation of proteins (or domains thereof), or of smaller peptides. The protein into which mutations are introduced may be referred to as a “reference protein” (this does not mean that it is disclosed in a prior art reference”) and the resulting protein as the “mutant protein”. The reference protein may itself be a mutant of a naturally occurring protein. The term “protein” applies mutatis mutandis to oligopeptides, and to domains of proteins.

First, the mutated entity may be one involved in the initial screen. The mutated sequence may correspond to a receptor (including both endogenous and chimeric receptors), a ligand for a receptor (including the ligand-like moiety of a hybrid protein), or a component of a signal producing system. The latter may be, for example, a DNA-binding or transactivation domain of a transcription factor, a reporter (or fragment thereof), or a “downstream” protein component of the signal producing system.

A target-binding member of the screened library may also be mutated. In turn, desirable mutants may be further mutated.

In some instances, the invention also contemplates mutation of nucleic acids, for example, the target DNA operator for the DNA-binding domain of a transcription factor.

In preferred embodiments, the mutant protein is “substantially identical”, as hereafter defined, to a reference protein with a desired binding or biological activity.

The mutant protein may also be a hybrid (chimera) of at least one domain of each of two more reference proteins (or domains), as hereafter discussed. It may be, for example, a hybrid of a domain from a protein A, and a domain from protein. B. These domains may be identical to the original domains, or mutants thereof.

“Substantially Identical”

A mutant protein (domain, peptide) is substantially identical to a reference protein (domain, peptide) if (a) it has at least 10% of a specific binding activity or a non-nutritional biological activity of the reference protein (domain, peptide), and (b) (1) is at least 50% identical in amino acid sequence to the reference protein (domain, peptide), and/or (2) differs from the reference protein (domain, peptide) solely by one or more conservative modifications. If (1) applies, it may be said to “substantially percentagewise identical”. If (2) applies, it may be said to be “conservatively identical”. Both may apply.

Percentage amino acid identity is determined by aligning the mutant and reference sequences according to a rigorous dynamic programming algorithm which globally aligns their sequences to maximize their similarity, the similarity being scored as the sum of scores for each aligned pair according to an unbiased PAM250 matrix, and a penalty for each internal gap of −12 for the first null of the gap and −4 for each additional null of the same gap. The percentage identity is the number of matches expressed as a percentage of the adjusted (i.e., counting inserted nulls) length of the reference sequence.

A mutant DNA sequence is substantially identical to a reference DNA sequence if they are structural sequences, and encoding mutant and reference proteins which are substantially identical as described above.

If instead they are regulatory sequences, they are substantially identical if the mutant sequence has at least 10% of the regulatory activity of the reference sequence, and is at least 50% identical in nucleotide sequence to the reference sequence. Percentage identity is determined as for proteins except that matches are scored +5, mismatches −4, the gap open penalty is −12, and the gap extension penalty (per additional null) is −4.

Preferably, nucleotide sequences which are substantially identical exceed the minimum identity of 50% e.g., are 51%, 66%, 75%, 80%, 85%, 90%, 95% or 99% identical in sequence.

DNA sequences may also be considered “substantially identical” if they hybridize to each other under stringent conditions, i.e., conditions at which the Tm of the heteroduplex of the one strand of the mutant DNA and the more complementary strand of the reference DNA is not in excess of 10° C. less than the Tm of the reference DNA homoduplex. Typically this will correspond to a percentage identity of 85-90%.

“Conservative Modifications”

“Conservative modifications” are defined as

(a) conservative substitutions of amino acids as hereafter defined; or

(b) single or multiple insertions (extension) or deletions (truncation) of amino acids at the termini.

“Semi-Conservative Modifications” are modifications which are not conservative, but which are (a) semi-conservative substitutions as hereafter defined; or (b) single or multiple insertions or deletions internally, but at interdomain boundaries, in loops or in other segments of relatively high mobility. Preferably, all nonconservative modifications are semi-conservative.

The term “conservative” is used here in an a priori sense, i.e., modifications which would be expected to preserve 3D structure and activity, based on analysis of the naturally occurring families of homologous proteins, the chemical similarity of the amino acids in question, and past experience with the effects of deliberate mutagenesis, rather than post facto, a modification already known to conserve activity. Of course, a modification which is conservative a priori may, and usually is, also conservative post facto.

Preferably, except at the termini, no more than about five amino acids are inserted or deleted at a particular locus, and the modifications are outside regions known to contain binding sites important to activity.

Preferably, insertions or deletions are limited to the termini. More preferably, there are no indels; the modifications are just conservative substitutions.

A conservative substitution is a substitution of one amino acid for another of the same exchange group, the exchange groups being defined as follows

I Gly, Pro, Ser, Ala (Cys) (and any nonbiogenic, neutral amino acid with a hydrophobicity not exceeding that of the aforementioned a.a.'s)

II Arg, Lys, His (and any nonbiogenic, positively-charged amino acids)

III Asp, Glu, Asn, Gln (and any nonbiogenic negatively-charged amino acids)

IV Leu, Ile, Met, Val (Cys) (and any nonbiogenic, aliphatic, neutral amino acid with a hydrophobicity too high for I above)

V Phe, Trp, Tyr (and any nonbiogenic, aromatic neutral amino acid with a hydrophobicity too high for I above).

Note that Cys belongs to both I and IV.

Residues Pro, Gly and Cys have special conformational roles. Cys participates in formation of disulfide bonds. Gly imparts flexibility to the chain. Pro imparts rigidity to the chain and disrupts α helices. These residues may be essential in certain regions of the polypeptide, but substitutable elsewhere.

One, two or three conservative substitutions are more likely to be tolerated than a larger number.

“Semi-conservative substitutions” are defined herein as being substitutions within supergroup I/II/III or within supergroup IV/V, but not within a single one of groups I-V. They also include replacement of any other amino acid with alanine. If a substitution is not conservative, it preferably is semi-conservative.

“Non-conservative substitutions” are substitutions which are not conservative. They include “semi-conservative substitutions” as a subset.

“Highly conservative substitutions” are a subset of conservative substitutions, and are exchanges of amino acids within the groups Phe/Tyr/Trp, Met/Leu/Ile/Val, His/Arg/Lys, Asp/Glu and Ser/Thr/Ala. They are more likely to be tolerated than other conservative substitutions. Again, the smaller the number of substitutions, the more likely they are to be tolerated.

A protein (peptide) is conservatively identical to a reference protein (peptide) it differs from the latter, if at all, solely by conservative modifications, the protein (peptide remaining at least seven amino acids long if the reference protein (peptide) was at least seven amino acids long.

A protein is at least semi-conservatively identical to a reference protein (peptide) if it differs from the latter, if at all, solely by semi-conservative or conservative modifications.

A protein (peptide) is nearly conservatively identical to a reference protein (peptide) if it differs from the latter, if at all, solely by one or more conservative modifications and/or a single nonconservative substitution.

It is highly conservatively identical if it differs, if at all, solely by highly conservative substitutions.

The core sequence of a reference protein (peptide) is the largest single fragment which retains at least 10% of a particular specific binding activity, if one is specified, or otherwise of at least one specific binding activity of the referent. If the referent has more than one specific binding activity, it may have more than one core sequence, and these may overlap or not.

If it is taught that a peptide of the present invention may have a particular similarity relationship (e.g., markedly identical) to a reference protein (peptide), preferred peptides are those which comprise a sequence having that relationship to a core sequence of the reference protein (peptide), but with internal insertions or deletions in either sequence excluded. Even more preferred peptides are those whose entire sequence has that relationship, with the same exclusion, to a core sequence of that reference protein (peptide).

The Biokeys of the present invention include not only the listed (reference) peptides, but also other peptides which are markedly identical. Preferably, the degree of identity (similarity) is higher than merely markedly identical.

Where this specification sets forth a consensus sequence for a particular class of peptides then any peptide comprising said consensus is a preferred peptide according to this invention.

“Non-Naturally Occurring”

Reference to a peptide or protein as “non-naturally occurring” means that it does not occur, as a unitary molecule, in non-genetically engineered cells or viruses. It may be biologically produced in genetically engineered cells, or genetically engineered virus-transfected cells, and it may be a segment of a larger, naturally occurring protein.

If it is disclosed that a peptide preferably is not naturally occurring, it more preferably is not conservatively identical to any naturally occurring peptide.

Design of Functional Mutants, Generally

A protein is more likely to tolerate a mutation which

(a) is a substitution rather than an insertion or deletion;

(b) is an insertion or deletion at the terminus, rather than internally, or, if internal, is at a domain boundary, or a loop or turn, rather than in an alpha helix or beta strand;

(c) affects a surface residue rather than an interior residue;

(d) affects a part of the molecule distal to the binding site;

(e) is a substitution of one amino acid for another of similar size, charge, and/or hydrophobicity, and does not destroy a disulfide bond or other crosslink; and

(f) is at a site which is subject to substantial variation among a family of homologous proteins to which the protein of interest belongs.

These considerations can be used to design functional mutants.

Surface vs. Interior Residues

Charged residues almost always lie on the surface of the protein. For uncharged residues, there is less certainty, but in general, hydrophilic residues are partitioned to the surface and hydrophobic residues to the interior. Of course, for a membrane protein, the membrane-spanning segments are likely to be rich in hydrophobic residues.

Surface residues may be identified experimentally by various labeling techniques, or by 3-D structure mapping techniques like X-ray diffraction and NMR. A 3-D model of a homologous protein can be helpful.

Binding Site Residues

Residues forming the binding site may be identified by (1) comparing the effects of labeling the surface residues before and after complexing the protein to its target, (2) labeling the binding site directly with affinity ligands, (3) fragmenting the protein and testing the fragments for binding activity, and (4) systematic mutagenesis (e.g., alanine-scanning mutagenesis) to determine which mutants destroy binding. If the binding site of a homologous protein is known, the binding site may be postulated by analogy.

Protein libraries may be constructed and screened that a large family (e.g., 108) of related mutants may be evaluated simultaneously.

Design of Chimeric Proteins

A chimeric protein is a hybrid of two or more different proteins (or recognizable portions thereof). The component proteins (which are in effect, domains of the chimeric protein) may be naturally occurring as independent entities, or mutants of naturally occurring proteins, or fragments thereof. The proteins are usually related, e.g., a statistically significant (at least 6 sigma) alignment when aligned as described above, and compared to the similar alignment of jumbled sequences. More often they are “substantially identical” as defined above.

Functional chimeras may be identified by a systematic synthesize-and-test strategy. It is not necessary that all theoretically conceivable chimeras be evaluated directly.

One strategy is described schematically below. We divide the aligned protein sequences into two or more testable units. These units may be equal or unequal in length. Preferably, the units correspond to functional domains or are demarcated so as to correspond to special features of the sequence, e.g., regions of unusually high divergence or similarity, conserved or unconserved regions in the relevant protein family or the presence of a sequence motif, or an area of unusual hydrophilicity or hydrophobicity. Let “A” represent a unit of protein A, and “B” a corresponding unit of protein B. If there are five units (the choice of five instead of two, three, four, six, ten, etc. is arbitrary), we can synthesize and test any or all of the following chimeras, which will help us rapidly localize the critical regions:

(a) progressive C-terminal substitution of B sequence for A sequence, e.g.,

A A A A A
A A A A B
A A A B B
A A B B B
A B B B B
B B B B B

(b) progressive N-terminal substitution of B sequence for A sequence

A A A A A
B A A A A
B B A A A
B B B A A
B B B B A
B B B B B

(c) dual terminal substitutions, e.g.,

B B B B B
A B B B A
A A B A A
A A A A A

and

A A A A A
B A A A B
B B A B B
B B B B B,

and

(d) single replacement “scans,” such as

B A A A A
A B A A A
A A B A A
A A A B A
A A A A B
and
A B B B B
B A B B B
B B A B B
B B B A B
B B B B A

Based on the data these tests provide, it may appear that, e.g., the key difference between the A and B sequences vis-a-vis a property of interest, is in the fifth unit. One can then subdivide that unit into subunits and test further, e.g.

| | |
|---|---|
| B B B B | (bb) |
| B B B B | (ba) |
| B B B B | (ab) |
| B B B B | (aa) |

where the parenthesis refer to two subunits into which the fifth unit was subdivided.

General Method of “Fingerprinting”

In essence, a panel of “BioKeys” (receptor conformation-sensitive receptor binding molecules, typically peptides) which alter the conformation of a receptor in distinctly different ways, may be used to obtain a “fingerprint” of how a compound of interest interacts with that receptor in its various BioKey-modified conformations, each element of the fingerprint being a measure of the strength of interaction of the compound with the receptor in the presence of a given BioKey. Once fingerprints are obtained for a reasonable number of reference compounds with known biological activities, preferably as measured by a “gold standard” (whole animal, or isolated organ or tissue) assay, the similarity of the fingerprint of a new compound to that of the reference compounds may be calculated, and used to predict the bioactivity of the new compound.

The invention has advantages over whole animal-based assay systems in that 1) the same technology can be applied to a variety of different receptors, 2) the system can be used for high throughput screening and compound characterization, and 3) the system gives very distinct patterns for agonists and antagonists of receptor activity using very little protein.

Thus, in the present invention, the biological activity of a test substance, as mediated by a particular receptor, in a particular organism, and thereof is predicted by:

(I) providing a panel of “Biokeys”, the “Biokeys” having a differential ability to bind the receptor in the presence or absence of one or more ligands, said panel therefore being able to discriminate among two or more different receptor conformations,

(II) screening a set of two or more reference substances, which are known pharmacological agonists or antagonists of the receptor in one or more organisms and tissues, for the ability to alter the binding of the “Biokeys” to the receptor, thereby obtaining a reference “fingerprint”, for each reference substance, which is an array of descriptors, each descriptor defining, qualitatively or quantitatively, the effect of the reference compound on the binding of a Biokey panel member to the receptor.

(III) The test compound is similarly screened for its ability to alter the binding of the “Biokeys” to the receptor, thereby obtaining a test fingerprint,

(IV) the similarity of the test fingerprint to each of the reference fingerprints is determined, and

(V) the biological activity of the test substance in one or more target organisms, and in one or more target tissues thereof, is predicted on the basis of the biological activities of the reference substances therein, appropriately weighted by the similarity between the test substance and the reference substance.

The Biokey panel of step (I) is preferably obtained by screening the members of a combinatorial library for the ability to bind to (a) the unliganded receptor, and (b) a liganded receptor. In one embodiment, a combinatorial library is first screened against (a), and then either the whole library, or only the unliganded receptor-binding members, are screened against (b). In another embodiment, the whole library is screened against (a) and (b) simultaneously. It is also permissible to screen first against (b) and then against (a).

In the cross-referenced applications PCT/US99/06664 and Ser. No. 09/429,331, we described a variety of means of obtaining the Biokey panel of step (I). However, in this application, we will assume that one or more members of the panel are obtained by use of the aforementioned combinatorial library screened by a cell-based assay. It is not necessary that all of the panel members be identified in this way.

It will be appreciated that step (II) need only be performed once for a given receptor and that it is not necessary that all reference substances be fingerprinted simultaneously. Also, steps (II) and (III) may be interchanged.

In step (IV), similarity may be determined in a qualitative and subjective way, i.e., by “eyeballing” the fingerprints and judging from experience which is more similar, or in a quantitative and objective manner, using the similarity measures set forth infra.

Similarly, in step (V), the biological activity may be predicted in a qualitative and subjective way, or more quantitatively and objectively, by mathematically weighting each reference substance's activity scores by the calculated similarity of its fingerprint to the fingerprint of the test substance.

In a prior application, two different “fingerprinting” embodiments were described.

In the “molecular braille” (MB) embodiment of the invention, the reference and test fingerprints are based on in vitro (cell-free) assays.

In the “cellular-braille” (CB) embodiment, the reference and test fingerprints are based on cellular assays (but not on assays of whole multicellular organisms, or their organs or tissues).

The advantages of “molecular braille” are

gives information about affinity, and, based on a fingerprint, bioactivity in a single assay

can be faster and less expensive if the protein is a) inexpensive to purchase or b) easy to express and purify

gives information about structure-activity relationships

peptide/receptor interactions may be more sensitive because there will not be anything extraneous to get in the way

Its disadvantages are

protein may not be properly folded, modified, or be in the presence of cofactors it needs to be active

doesn't give much of the information given by CB

In contrast, the advantages of “cellular braille” are

If in yeast it can be cheaper than MB

Bioactivity (including dose:effect) information

gives closer indication of how a whole animal might respond

you may get active metabolites

no need for protein purification

Its disadvantages are

compounds that cannot get into the cell will automatically be selected against does not give affinity information directly

throughput likely to be lower than with MB, although still better than whole animal assay.

Both “molecular braille” and “cellular braille” are faster and less expensive than whole animal bioassays, and more readily automated for high throughput, and their use as preliminary screens helps minimize experimentation on animals, which itself is an ethical goal of society.

It will be appreciated that both techniques may be used, either sequentially or simultaneously. For example, MB may be used as a first screen and CB as a second screen of the first round positives. Or compounds may be screened by both MB and CB, and compounds earmarked by either screen given further attention. Similarities may be calculated separately from in vitro and cell-based assays, or the results of these two types of assays may be combined into a single fingerprint for each reference or test compound.

The present invention merely requires that at least one of the peptides used in at least one “fingerprinting panel” MB or CB, be a peptide from a peptide library coexpressed with a receptor, and found to bind that receptor, as described above.

BioKeys are probes for alterations in receptor conformation, and can readily distinguish between active, inactive and partially active receptor. The patterns of binding obtained with the peptides provides a fingerprint of the receptor conformation. The binding of the individual peptides will increase or decrease in the presence of an agonist or an antagonist of receptor activity. Such activity may or may not be tissue-specific. In some cases, whether a molecule is an agonist or an antagonist will depend on the tissue in question (e.g. for SERMs), or on other environmental factors. Therefore, the peptides may be used to classify compounds, not only as pure agonists or antagonists, but also more complexly. The method has the following applications:

1) One or more of these peptides can be used in a competitive displacement assay to identify modulators of receptor activity in a high-throughput (in vitro or simple cell) screen.

2) The peptides can be used to fingerprint modulators of receptor activity and classify them as agonists or antagonists of receptor activity.

3) Peptides identified for orphan receptors may be used to identify the natural ligand of these receptors.

4) This method may be used for nuclear receptors as well as other receptors such as G-protein coupled receptors.

5) Method can be applied to any protein that undergoes a conformational change upon ligand/substrate binding.

In a particular preferred embodiment, the invention is used to predict SERM activity against nuclear receptors, such as the estrogen receptor.

In order to characterize SERM activity at the estrogen receptor, we have developed a system that utilizes peptides to mimic the binding of various ER associated proteins to ER α and β in an in vitro setting. The peptides bind preferentially to either the active or inactive conformation of the receptor, and will distinguish between different conformational changes in the ER that result from the binding of a SERM. The system will also allow the comparison of effects of the SERM on ER α and β. This assay provides a simple procedure to determine the relative agonist/antagonist activity of a newly identified SERM. The technology may also be applied to the analysis of selective modulators of any receptor.

Certain sites on the receptor are only available for binding when an agonist is bound to the ER. Other sites are more readily available for binding with a SERM complexed ER. The relative binding affinities of these peptides on an estrogen completed receptor, or a SERM complexed receptor relative to an unliganded receptor provides a fingerprint that is indicative of the agonist/antagonist activity of the SERM. Agonists of receptor function and SERMs produced distinct fingerprints in our system indicative of their distinct in vivo functions. This system may be used as a primary screening tool to identify hits, to classify lead compounds from a drug screen, to characterize SERMs in terms of agonist and antagonist function and to predict possible clinical effects of SERMs such as tissue and receptor specificity. This method can also be applied to the fractionation of mixtures of SERMs to determine which components are producing agonistic and antagonistic activity. This method may also be used with other receptors (e.g., progesterone, androgen, glucocorticoid, thyroid, vitamin D, beta-adrenergic, dopamine, epidermal growth factor, etc.), to identify, characterize and classify modulators of receptor activity.

While peptides have been identified for use as probes to modify receptor conformation, to help screen compound libraries, certain of these peptides may be useful in their own right as drugs or diagnostics.

In addition, nonpeptide mimetics or other analogues of the aforementioned peptides may be useful as drugs or diagnostics.

The screened compounds, and their analogues, are also of interest.

Substances

A “substance” may be either a pure compound, or a mixture of compounds. Preferably it is at least substantially pure, that is, sufficiently pure enough to be acceptable for clinical use. Whether pure or not, the test sample of the substance comprises at least an effective amount (i.e., able to give rise to a detectable biological response in a biological assay) of a biologically active compound, or it comprises a substantial amount of a compound which is suspected of being biologically active and is suitable as a drug lead if so active.

Test substances and Drug Leads

A test substance comprises an effective amount of a compound, which is a member of a structural class which is generally suitable, in terms of physical characteristics (e.g., solubility), as a source of drugs and which is not known to have the pharmacological activity of interest.

Biokeys

For the purpose of the present invention, Biokeys are substances whose ability to bind to a target receptor in the presence or absence of one or more reference ligands for that receptor can be used to differentiate the reference ligands, and ultimately to calculate the degree of similarity between a test substance (having an assayable effect on the binding of the Biokeys to the target receptor protein) and reference substances (likewise having an assayable effect as such binding, but whose effect on biological activity of the receptor protein in target organisms and tissues of interest is also known).

Preferably, Biokeys are members of a combinatorial library, and in particular an amplifiable combinatorial library such as a peptide or nucleic acid library. The library may then be screened for binding to various receptor conformations. Biokeys need not themselves be suitable as drug leads.

A number of Biokeys have already been identified for the estrogen receptor (see tables 1-4,7-10, 14A-14B, 15A, 15B, 101) by in vitro, nonbiological screening of peptide libraries.

Other Biokeys have been identified for the estrogen receptor (table 501) and for the androgen receptor (tables 502A, 502B) by the cell-based screening of peptide libraries as here contemplated.

Biokey Panel

For the purpose of fingerprinting the reference and test substances, a representative selection of Biokeys are collected into a panel. If only a single reference ligand is known for a receptor, the panel could include one or more representative members of each of at least two of the following binding classes:

| | | Change in Binding |
|---|---|---|
| Class | Binds UL-R | (Effect of Ligand) |
| A | + | + |
| B | + | − |
| C | + | 0 |
| D | − | 0 |
| E | − | + |

Thus classes A, B and C bind unliganded receptor (UL-R), but the ligand increases the binding of A, decreases the binding of B, and has no effect on the binding of C. Classes D and E do not bind the UL-R. The ligand causes E, but not D, to bind the receptor.

Instead of only two of the above, the panel can include representative members of three, four or all five of the classes, if Biokeys having the appropriate properties can be identified.

The above classes look at binding in only a qualitative manner. However, it would be possible to differentiate between strong and weak binders of UL-R, and between large and small changes in binding as the result of the ligand. If desired, one could draw even finer divisions, e.g.; strong vs. moderate vs. weak, etc.

If more than one ligand is available, the combinatorial possibilities are increased, and, if suitable Biokeys can be identified, the panel can be expanded appropriately.

For example, with two ligands, the following possibilities could exist

| Biokey | UL-R | Ligand A | Ligand B |
|---|---|---|---|
| Z | + | + | + |
| Y | + | + | 0 |
| X | + | + | − |
| W | + | 0 | + |
| V | + | 0 | 0 |
| U | + | 0 | − |
| T | + | − | + |
| S | + | − | 0 |
| R | + | − | − |
| Q | − | 0 | 0 |
| P | − | + | 0 |
| O | − | 0 | + |
| N | − | + | + |

And one could discriminate further, e.g., for Z-1, the effect of A is greater than that of B, for Z-2, the reverse, and for Z-3, the effects are equal.

Preferably, one, two, three, four, five or more reference ligands are used to define the Biokey panel.

It is not necessary that a particular binding class be represented by only a single Biokey. Instead, it may be represented by a mixture of two or more Biokeys, and indeed the mixture may correspond to all of the Biokeys in the Biokey library which satisfied the binding criteria for the class in question.

The members of the Biokey panel are chosen with a view to maximizing the discriminatory power of the panel. For example, to take an extreme case, if two members of the panel have identical binding properties, vis-a-vis, all the available reference conformations of the receptor, then one of these members is redundant. While including it in the panel does no harm, it needlessly increases the costs of the screening.

The similarity of any pair of potential panel members may be determined using the similarity measures set forth infra. The overall diversity of a given panel may be determined by computing all of the pairwise dissimilarities. For a given size panel, extracted from a given library, one may seek to maximize the overall diversity of effect on biological activity. Or one may seek to determine, for a set of binding members from a library, what is the size and composition of the subject which maximizes the ratio of the overall diversity to the number of members.

The number of panel-based descriptors in the fingerprint will normally be equal to the number of members in the panel. The optimal number of members depends on the number of reference substances, and the ability of the panel to differentiate them. The larger the number of reference substances, and the larger the number of target organisms and tissues in which the biological activity of the reference substance is to be predicted, the larger the panel should be. Typically, there will be 2, 3, 4, 5, 6, 7, 8, 9, or 10 panel members. More members may be used, but the cost of the assay increases, without necessarily providing a commensurate increase in the predictive power of the data.

Reference substances

Reference substances are known pharmacological agonists or antagonists for the receptor in question, and have a known or ascertainable biological activity in one or more organisms and/or tissues.

Typically, for a given receptor, one, two, three, four, five or more reference substances will be fingerprinted.

“Fingerprinting” of Test and Reference Substances

Each test substance will be characterized by a plurality of descriptors (the “fingerprint”) by which it may be compared to reference substances.

These reference substances may be the particular reference ligands used to define the Biokey panel, but are not limited to those reference ligands. Thus, in example 1, only estradiol was used to define the five classes of peptides, but the reference substances were estradiol, estriol, tamoxifen, nafoxidine and clomiphene. The use of estradiol was not critical; the reference substances need not include any of the reference ligands used to define the BioKey panel.

The reference substances must be pharmacological agonists or antagonists in at least one organism and tissue, while the reference ligands are not so limited.

For the purpose of the present invention, a plurality of descriptors must refer to the effect of the test substance on the binding of a member of the Biokey panel to a reference conformation, e.g., unliganded receptor X, receptor X/ligand A, receptor X/ligand B, unliganded receptor Y, receptor Y/ligand C, etc. Note that in this context, the term “member” may refer to a mixture of Biokeys of the same binding class. The descriptor may be qualitative (binds vs. nonbinds; increases vs. decreases vs. no effect, etc.) or quantitative. Preferably, at least 2-10 Biokey-based descriptors are used.

The test substance may additionally be characterized by other descriptors, such as structural descriptors, known in the art. Preferably, at least 5-10 different reference substances are “fingerprinted”.

The reference substances will be characterized in a similar manner to the test substances, so that their descriptors may be “paired” with the test substance descriptors in such a manner that the degree of similarity may be calculated.

When fingerprinting a given reference or test substance, it may be screened simultaneously against all panel members, or individual panel members (or subsets of panel members) may be tested separately. Also, all reference substances may be screened simultaneously against a given receptor/panel member combination, or the reference substances may be screened individually. The same is true of the screening of the test substances. The test substances may be screened after, before or simultaneously with the reference substances.

Descriptors

A “descriptor” (also known as a parameter, character, variable, or variate) is a numerically expressed characteristic of a compound (which may be a protein, or a protein ligand), which helps to distinguish that compound from others. A descriptor value need not be absolutely specific to a compound to be useful. The characteristics may be pure structural characteristics (as in a “structural descriptor”) or they may refer to the compound's interaction with other compounds. “Paired Descriptors” are descriptors of the same property as measured in two different molecules. A “descriptor array”, “list”, or “set” is an array, list or set whose elements are different descriptors for the same molecule. Such an array, list or set is referred to herein as a “fingerprint”.

A plurality of paired descriptors for two compounds may be used to calculate a similarity between the two compounds.

Similarity Measures

A similarity measure or coefficient quantifies the relationship between two individuals (compounds), given the values of a set of variates (descriptors) common to both. Similarity coefficients are usually defined to take values in the range of 0 to 1.

One commonly used measure of similarity is the product moment correlation coefficient. Its correlation is unity whenever two profiles are parallel, regardless of how far apart they are in level. Two profiles may have correlation of +1 even if they are not parallel, provided that the two sets of scores are linearly related.

Descriptors may be quantitative or qualitative. Quantitative descriptors may be integers or real numbers. Qualitative descriptors divide the data into categories which may be, but need not be, expressible as having relative magnitudes. Binary descriptors are a special case of qualitative descriptors, in which there are just two categories, typically representing the presence or absence of a feature. Qualitative data for which the variates have several levels may be treated like binary data with each level of a variate being regarded as a single binary variable (i.e., an eight level variate expressed as eight bits). Or the levels may be numbered sequentially (i.e., an eight level variable expressed as three bits).

A set of n-descriptors defines an n-dimensional descriptor space; each compound for which a descriptor set is available may be said to occupy a point in descriptor space. The dissimilarity of two compounds may be expressed as a distance between the two points which they occupy in descriptor space.

A distance measure is a similarity measure which is also a metric, i.e., satisfies the conditions (i) d(x,y)≧0; and d(x,y)=0 if x=y; (ii) d(x,y)+d(y,x); and (iii) d(x,z)+d(y,z)≧d(x,y) (the metric or triangular inequality). Of course, the greater the distance, the less the similarity.

Distances may be calculated on the basis of any of a variety of distance measures known in the statistical arts.

The most commonly used distance measure is the Euclidean metric:

$${d}_{i j} = {( {\sum }_{K} {( {X}_{i k} - {X}_{j k} )}^{2} )}^{1 / 2}$$

It corresponds most closely to our intuitive sense of distance.

A distance measure may be transformed into a similarity measure by any of a variety of transformations that convert a non-negative number to the range 0.1, e.g.,

Sᵢⱼ=1/(1+dᵢⱼ)

A similarity measure may be converted into a distance by, e.g., dᵢⱼ=1-sᵢⱼ.

If there is a theoretical maximum distance (dₜₘₐₓ), based on the theoretically possible ranges for each of the component descriptors, the similarity may be expressed as

Sᵢⱼ=1−(dᵢⱼ/dₜₘₐₓ)

Alternatively, one may calculate the distances between all pairs, and then use the actual maximum distance (dₐₘₐₓ)

Sᵢⱼ=1−(dᵢⱼ/dₐₘₐₓ)

Instead of using the ratio of the actual distance to the actual or theoretical maximum distance, one may express sᵢⱼ as the fraction of the pairs for which the distance is areater than or equal to dᵢⱼ. This is a measure of relative similarity.

Descriptors may be weighted (or otherwise transformed) for any of several reasons, including:

(a) to reflect the perceived value of the descriptor for determining whether two proteins will be modulated by structurally similar drugs;

(b) to reflect the perceived reliability of the descriptor data;

(c) to correct for differences in scale between descriptors, so that a descriptor does not dominate a similarity or distance calculation merely because its values are of higher magnitude or are spread over a greater range; and

(d) to correct for correlations between descriptors.

The raw descriptor values may be, but need not be, transformed prior to use in calculating distances. Typical transformations are (a) presence (1)/absence (0), (b) 1n (x+1), (c) frequency in sample, (d) root, and (e) relative range, i.e., (value-min.)/(max-min).

The raw descriptor values may be standardized (normalized) to have zero mean (x′=x−μₓ) and/or unit variance (x′=x/σₓ), possibly both (x′=(x−μₓ)/σₓ) or they be standardized (unitized) to fall into the range 0 to 1.

Descriptor weights may be adjusted empirically on the basis of specially designed test sets. A training set of proteins is identified. Descriptors are evaluated for each protein in the set. A training set of compounds, including are also tested against each compound in the set. These compounds are chosen so that, for any protein in the set, there is at least one compound which is an agonist or antagonist for it. A neural net, with the descriptor weights as inputs, is used to predict the activity of each compound against each protein, using the calculated protein similarities. For example, it will calculate the similarity of protein x to all other proteins, then treat the activities of the compounds against the other proteins as “knowns” and use it to predict the activity of the compounds against protein x. This is done repeatedly, with each protein taking on the role of protein x, in turn.

The coefficient of variation may be useful in comparing descriptors; it is the standard deviation divided by the mean. If there is no information available about the ultimate significance of a descriptor, one may give a greater weight to descriptors which have a larger CV and hence a more uniform distribution.

It must be emphasized that we do not require use of weighted descriptors, let alone of any particular method of deriving weights.

It is likely that some degree of correlation will exist among the descriptors. Standard mathematical methods, such as cluster analysis, principal components analysis, or partial least squares analysis, may be used to determine which descriptors are strongly correlated and to replace them with a new descriptor which is a weighted sum of the original correlated descriptors. One may alternatively choose (perhaps randomly) one of each pair of highly correlated descriptors and simply prune it, thereby reducing the amount of data which must be collected.

One way of correcting for correlation among the descriptors is for each descriptor m, calculate the average of its squared correlation coefficients with all descriptors n (including m=n, for which the coefficient is necessarily unity), and subtract this number from one to obtain a weight representing the fraction of the variation in descriptor m which is not explained by the “average” descriptor n. With this “average r²” method, if we have four descriptors, and two are perfectly correlated to each other, and the descriptors are otherwise completely uncorrelated, the correlated descriptors will have weights of 0.5 each, and the other two will have weights of 1.0 each.

The diversity of a set of compounds, as measured by a set of descriptors, may be calculated in several ways.

A purely geometric method involves assuming that each compound sweeps out a hypersphere in descriptor space, the hypersphere having a radius known as the similarity radius. The total hypervolume in descriptor space of points within a unit similarity radius of one or more of the compounds is calculated. This is compared to the hypervolume achievable if none of hypersphere's overlap; i.e., to n * volume of a single hypersphere, where n is the number of compounds in the set. The swept hypervolume may be determined exactly, or by Monte Carlo methods. The ratio of the swept hypervolume to the maximum hypervolume is a measure of compound set diversity, ranging from 1 (maximum) to 1/n (minimum).

Another approach is to calculate all of the pairwise distances between compounds in descriptor space. The mean distance is a measure of diversity. If desired, this can be scaled by calculating the ratio of the mean distance to the maximum theoretical distance.

A third approach is to apply cluster analysis to the set of compounds. The method used should be one which does not set the number of clusters arbitrarily, but rather decides the number based on some goodness-of-fit criterion. The resulting number of cluster is a measure of diversity, as is the ratio of the number of clusters to the number of compounds.

One may calculate a measure of disorder for a descriptor as

$$H ( k ) = - {\sum }_{g = 1}^{{m}_{k}} {P}_{k g} ln {P}_{k g}$$

where mₖ is the number of different states in descriptor k, and P_{kg} is the observed proportion of individuals exhibiting state g for descriptor k. For uncorrelated descriptors, the sum of H(k) for all k is a measure of overall diversity. Standard techniques may be used to correct for correlation.

Preliminary Screening Assays

The invention contemplates at least three occasions for preliminary screening during “Fingerprinting”:

(a) screening for potential “BioKeys”, using a known receptor (or ligand-binding moiety thereof) and one or more known pharmacological modulators of the receptor (see General Method of Fingerprinting, step (I)),

(b) screening reference compounds, having a known receptor-mediated bioactivity using a known receptor and an established BioKey panel, to obtain reference fingerprints (see General Method of Fingerprinting, step (II), and

(c) screening test compounds for their ability to alter the binding of a panel of BioKeys to the receptor, thereby obtaining a test fingerprint (see General Method of Fingerprinting, step (III)).

The same or different screening methods may be used on each occasion.

Preliminary screening assays will typically be either in vitro (cell-free) assays (for binding to an immobilized receptor) or cell-based assays (for alterations in the phenotype of the cell). They will not involve screening of whole multicellular organisms, or isolated organs. The comments on biological assays apply mutatis mutandis to preliminary screening cell-based assays.

Thus, in screening for each of (a)-(c) above, a target receptor, one may use either an in vitro assay or a cell-based assay. In the latter case, yeast and mammalian assays are of particular interest. In (a), any of these assays may be used to screen a combinatorial peptide library.

BioKeys are identified by screening receptor-binding molecules for the ability to bind the receptor more strongly in one receptor conformation than in another receptor conformation. The possible receptor conformations include the unliganded receptor, receptor:agonist or receptor:antagonist pairs, and other receptor:binding molecule complexes. Some receptors may participate in ternary or higher complexes which yield additional conformations. Thus, BioKeys may be identified by a Plurality of screens where the same peptides are screened for binding to the same receptor, but the receptor conformation varies from screen to screen. The screens may be simultaneous or sequential. The screens may be carried out each time on the same library, as a whole. Or the first screen may be performed on the whole library but the later screens on only a subset thereof, e.g., some or all of the successful binding molecules from the first screen.

While screening for potential BioKeys may be carried out in vitro or in a cell-based assay, the latter have the advantage that the receptor (if a cellular receptor) is in a more natural environment, and there is preselection for peptides that are stable intracellularly.

In the present application, it is contemplated that at least one BioKey of a panel will have been identified in step (I) by screening a combinatorial peptide library by a cell-based assay.

However, other members may have been identified by alternative screening assays.

Preferred In Vitro Screening Assays

Scintillation Proximity Assay (SPA):

An SPA is a homogeneous assay which relies on the short penetration range in solution of beta particles from certain isotopes, such as ³H, ²⁵I, ³³P and ³⁵S.

In a competitive SPA, the scintillant (which emits light when a beta particle passes close by) is conjugated to an analyte binding molecule. The analyte is allowed to compete with a short range beta particle-emitting radiolabeled analyte analogue for binding to the ABM. If the analyte analogue binds, the beta particles emitted by its label come close enough to stimulate the scintillant.

Usually, the scintillant is embedded in beads, or in the walls of the wells of a microtiter plate.

In a sandwich SPA, the scintillant-ABM conjugate binds the analyte, and a second radiolabeled ABM also binds the analyte, thereby forming a ternary complex.

There are practical reasons for using, instead of a scintillant-ABM conjugate, a primary simple ABM reagent, and a scintillant-(anti-ABM) conjugate acting as a secondary reagent which binds the primary reagent. The ABM of the primary reagent could then be a mouse monoclonal antibody, and the anti-ABM of the secondary reagent a cheaper polyclonal anti-mouse antibody, usable in assays for different analytes.

Fluorescence Polarization (FP): A method for detection of ligand binding that results in a change of the rotational relaxation time of the fluorescent label reflecting in a change in the total molecular mass of the complex containing the fluorescent ligand. A measurement is taken by excitation of the fluorescent moiety on the ligand by light of the proper wavelength that has passed through a polarizing filter and performing two measurements on the emitted light. The first measurement is performed by passing the light through a polarizing filter that is parallel to the polarization of the excitation polarizer. The second measurement is performed by passing the light through a polarizing filter that is perpendicular to the polarization of the excitation polarizer. The intensities of the emitted light from the parallel and perpendicular measurements are used to determine the polarization of the fluorescent ligand by the following equation mP=[(Iₚₐᵣₐₗₗₑₗ−I_{perpendicular})/(_{Iparallel}+I_{perpendicular})×1000] An increase in mP indicates that more polarized light is being emitted and corresponds to the formation of a complex.

Fluorescence Resonance Energy Transfer (FRET): A method for detection of complex formation, such as ligand-receptor binding, that relies upon the through-space interactions between two fluorescent groups. A fluorescent molecule has a specific wavelength for excitation and another wavelength for emission. Pairs of fluorophores are selected that have an overlapping emission and excitation wavelength. Paired fluorophores are detected by a through-space interaction referred to as resonance energy transfer. When a donor fluorophore is excited by light, it would normally emit light at a higher wavelength; however, during FRET energy is transferred from the donor to the acceptor fluorophore allowing the excited donor to relax to the ground-state without emission of a photon. The acceptor fluorophore becomes excited and release energy by emitting light at its emission wavelength. This means that when a donor and an acceptor fluorophore are held in close proximity (<100 Angstroms), such as when one fluorophore is attached to a ligand and one is attached to a receptor and the ligand binds to the receptor, excitation of the donor is coupled with emission from the acceptor. Conversely, if no complex is formed the excitation of the donor results in no emission from the acceptor. A common modification of this technique, sometimes referred to as fluorescence quenching, is accomplished using an acceptor group that is not fluorescent but efficiently accepts the energy from the donor fluorophore. In this case, when a complex is formed the excitation of the donor fluorophore is not accompanied by light emission at any wavelength. When this complex is dissociated the excitation of the donor results in emission of light at the wavelength of the donor.

Time-Resolved Fluorescence

The basic fluorescence assays can be modified to increase the signal to noise ratio. If there is a difference in the temporal behavior of signal fluorescence and background fluoresence, then “time-resolved fluorescence” may be used to better distinguish the two.

One may measure the decay of the total fluorescence intensity, or the decay of the polarization anisotropy.

In a time-resolved form of a FRET assay, Europium cryptate (EuK) serves as the donor fluorophore. The cryptate protects the europium ion from fluorescence quenching. The acceptor fluorophore is XL665, a modified allopycocyanine. The efficiency of FRET is 50% at a distance of 9 nm in serum, and the emission is at 665 nm. The XL665 emission is measured after a 50 microsec time delay (hence the name) which eliminates background (e.g., from free XL665 not stimulated by EuK). This is possible because the XL665 emission is relatively long-lived.

Fluorescence assays may be used in both cell-free and cell-based formats. Of course, for cell-based assays, the fluorophore labeled probes must be introduced into the cells in question.

For more information on fluorescence assays, see Szollosi, et al., Comm. Clin. Cytometry, 34:159-179 (1998); Millar, Curr. Op. Struct. Biol., 6:637-42 (1996); Mitra, et al., Gene, 173:13-17 (1996), Alfano, et al., Ann. N.Y. Acad. Sci., 838:14-28 (1998); Lundblad, et al., Mol. Endocrinol., 10:607-12 (1996); Gonzalez and Negulescu, Curr. Op. Biotechnology, 9:624-31 (1998). For bioluminescence assays, see Stables, et al., Anal. Biochem., 252:115-126 (1997).

Drug Leads

The term “drug lead”, as used herein, refers to a compound which is a member of a structural class which is generally suitable, in terms of physical characteristics (e.g., solubility), as a source of drugs, and which has at least some useful pharmacological activity, and which therefore could serve effectively as a starting point for the design of analogues and derivatives which are useful as drugs. The drug leads may be former test substances identified as active by the methods described herein.

The “drug lead” may be a useful drug in its own right, or it may be a compound which is deficient as a drug because of inadequate potency or undesirable side effects. In the latter case, analogues and derivatives are sought which overcome these deficiencies. In the former case, one seeks to improve the already useful drug.

Such analogues and derivatives may be identified by rational drug design, or by screening of combinatorial or noncombinatorial libraries of analogues and derivatives.

Preferably, a drug lead is a compound with a molecular weight of less than 1,000, more preferably, less than 750, still more preferably, less than 600, most preferably, less than 500. Preferably, it has a computed log octanol-water partition coefficient in the range of −4 to +14, more preferably, −2 to +7.5.

Test Substances

Test substances are usually potential pharmacological agonists or antagonists for the receptor in question. Thus, they are usually drug leads as described above.

Preferably the test substances are small organic molecules, e.g., molecules with a molecular weight of less than 500 daltons, which are pharmaceutically acceptable.

The test substances may be substances which have already been identified as having the ability to specifically bind the receptor. If the test substances are initially chosen on this basis, then it is preferably that they be derived from a combinatorial library so screened.

Additionally or alternatively they may be analogues of substances known to bind the receptor, especially substances known to mediate the biological activity of the receptor. These may include analogues of the peptides of the present invention.

Preferably, the test substances are:

(1) analogues of known pharmacological agonists or antagonists of the receptor of interest;

(2) pharmacological agonists or antagonists with receptors structurally (at least 25% identical in amino acid sequence in a statistically significant (≧6 sigma) alignment) or functionally similar to the receptor of interest; and/or

(3) ligands known to bind the receptor of interest in vitro (these ligands may be peptides identified by the cell-based screening of the present invention), or analogues of same.

In some preferred embodiments the test substances are of chemical classes amenable to synthesis as a combinatorial library. This facilitates identification of test compounds which bind the receptor in vitro (a Pre-screen) and the subsequent proliferation of related compounds for testing if a test substance proves of interest.

Chemical Nature of Test Substances

Many drugs fall into one or more of the following categories: acetals, acids, alcohols, amides, amidines, amines, amino acids, amino alcohols, amino ethers, amino ketenes, ammonium compounds, azo compounds, enols, esters, ethers, glycosides, guanidines, halogenated compounds, hydrocarbons, ketones, lactams, lactones, mustards, nitro compounds, nitroso compounds, organo minerals, phenones, quinones, semicarbazones, stilbenes, sulfonamides, sulfones, thiols, thioamides, thioureas, ureas, ureides, and urethans.

Without attempting to exhaustively recite all pharmacological classes of drugs, or all drug structures, one or more compounds of the chemical structures listed below have been found to exhibit the indicated pharmacological activity, and these structures, or derivatives, may be used as design elements in screening for further compounds of the same or different activity. (In some cases, one or more lead drugs of the class are indicated.)

hypnotics
higher alcohols (clomethiazole)
aldehydes (chloral hydrate)
carbamates (meprobamate)
acyclic ureides (acetylcarbromal)
barbiturates (barbital)
benzodiazepine (diazepam)
anticonvulsants
barbiturates (phenobarbital)
hydantoins (phenytoin)
oxazolidinediones (trimethadione)
succinimides (phensuximide)
acylureides (phenacemides)
narcotic analgesics
morphines
phenylpiperidines (meperidine)
diphenylpropylamines (methadone)
phenothiazihes (methotrimeprazine)
analgesics, antipyretics, antirheumatics
salicylates (acetylsalicylic acid)
p-aminophenol (acetaminophen)
5-pyrazolone (dipyrone)
3,5-pyrazolidinedione (phenylbutazone)
arylacetic acid (indomethacin)
adrenocortical steroids (cortisone, dexamethasone,
prednisone, triamcilone)
athranilic acids
neuroleptics
phenothiazine (chlorpromazine)
thioxanthene (chlorprothixene)
reserpine
butyrophenone (halopendol)
anxiolytics
propandiol carbamates (meprobamate)
benzodiazepines (chlordiazepoxide, diazepam,
oxazepam)
antidipressants
tricyclics (imipramine)
muscle/relaxants
propanediols and carbamates (mephenesin)
CNS stimulants
xanthines (caffeine, theophylline)
phenylalkylamines (amphetamine)
(Fenetylline is a conjunction of theophylline and
amphetamine)
oxazolidinones (pemoline)
cholinergics
choline esters (acetylcholine)
N,N-dimethylcarbamates
adrenergics
aromatic amines (epinephrine, isoproterenol,
phenylephrine)
alicyclic amines (cyclopentamine)
aliphatic amines (methylhexaneamine)
imidazolines (naphazoline)
anti-adrenergics
indolethylamine alkaloids (dihydroergotamine)
imidazoles (tolazoline)
benzodioxans (piperoxan)
beta-haloalkylamines (phenoxybenzamine)
dibenzazepines (azapetine)
hydrazinophthalazines (hydralazine)
antihistamines
ethanolamines (diphenhydramine)
ethylenediamines (tripelennomine)
alkylamines (chlorpheniramine)
piperazines (cyclizine)
phenothiazines (promethazine)
local anesthetics
benzoic acid
esters (procaine, isobucaine, cyclomethycaine)
basic amides (dibucaine)
anilides, toluidides, 2, 6-xylidides (lidocaine)
tertiary amides (oxetacaine)
vasodilators
polyol nitrates (nitroglycerin)
diuretics
xanthines
thiazides (chlorothiazide)
sulfonamides (chlorthalidone)
antihelmintics
cyanine dyes
antimalarials
4-aminoquinolines
8-aminoquinolines
pyrimidines
biguanides
acridines
dihydrotriazines
sulfonamides
sulfones
antibacterials
antibiotics
penicillins
cephalosporins
octahydronapthacenes (tetracycline)
sulfonamides
nitrofurans
cyclic amines
naphthyridines
xylenols
antitumor
alkylating agents
nitrogen mustards
aziridines
methanesulfonate esters
epoxides
amino acid antagonists
folic acid antagonists
pyrimidine antagonists
purine antagonists
antiviral
adamantanes
nucleosides
thiosemicarbazones
inosines
amidines and guanidines
isoquinolines
benzimidazoles
piperazines

For pharmacological classes, see, e.g., Goth, Medical Pharmacology: Principles and Concepts (C.V. Mosby Co.: 8th ed. 1976); Korolkovas and Burckhalter, Essentials of Medicinal Chemistry (John Wiley & Sons, Inc.: 1976). For synthetic methods, see, e.g., Warren, Organic Synthesis: The Disconnection Approach (John Wiley & Sons, Ltd.: 1982); Fuson, Reactions of Organic Compounds (John Wiley & Sons: 1966); Payne and Payne, How to do an Organic Synthesis (Allyn and Bacon, Inc.: 1969); Greene, Protective Groups in Organic Synthesis (Wiley-Interscience). For selection of substituents, see e.g., Hansch and Leo, Substituent Constants for Correlation Analysis in Chemistry and Biology (John Wiley & Sons: 1979).

Small Organic Compound Combinatorial Library

The small organic compound combinatorial library (“compound library”, for short) is a combinatorial library whose members are suitable for use as drugs if, indeed, they have the ability to mediate a biological activity of the target protein.

Peptides have certain disadvantages as drugs. These include susceptibility to degradation by serum proteases, and difficulty in penetrating cell membranes. Preferably, all or most of the compounds of the compound library avoid, or at least do not suffer to the same degree, one or more of the pharmaceutical disadvantages of peptides.

The design of a library may be illustrated by the example of the benzodiazepines. Several benzodiazepine drugs, including chlordiazepoxide, diazepam and oxazepam, have been used on anti-anxiety drugs. Derivatives of benzodiazepines have widespread biological activities; derivatives have been reported to act not only as anxiolytics, but also as anticonvulsants, cholecystokinin (CCK) receptor subtype A or B, kappa opioid receptor, platelet activating factor, and HIV transactivator Tat antagonists, and GPIIbIIa, reverse transcriptase and ras farnesyltransferase inhibitors.

The benzodiazepine structure has been disjoined into a 2-aminobenzophenone, an amino acid, and an alkylating agent. See Bunin, et al., Proc. Nat. Acad. Sci. USA, 91:4708 (1994). Since only a few 2-aminobenzophenone derivatives are commercially available, it was later disjoined into 2-aminoarylstannane, an acid chloride, an amino acid, and an alkylating agent. Bunin, et al., Meth. Enzymol., 267:448 (1996). The arylstannane may be considered the core structure upon which the other moieties are substituted, or all four may be considered equals which are conjoined to make each library member.

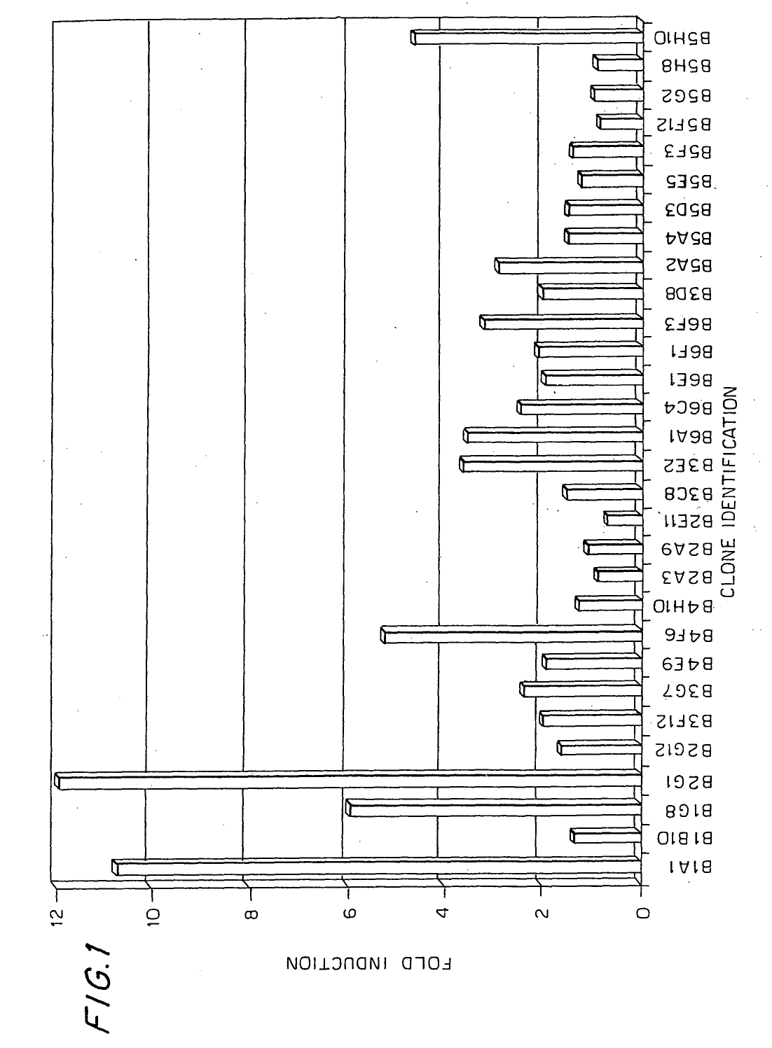

A basic library synthesis plan and member structure is shown in FIG. 1 of Fowlkes, et al., U.S. Ser. No. 08/740,671, incorporated by reference in its entirety. The acid chloride building block introduces variability at the R¹ site. The R² site is introduced by the amino acid, and the R³ site by the alkylating agent. The R⁴ site is inherent in the arylstannane. Bunin, et al. generated a 1,4-benzodiazepine library of 11,200 different derivatives prepared from 20 acid chlorides, 35 amino acids, and 16 alkylating agents. (No diversity was introduced at R⁴; this group was used to couple the molecule to a solid phase.) According to the Available Chemicals Directory (HDL Information Systems, San Leandro Calif.), over 300 acid chlorides, 80 Fmoc-protected amino acids and 800 alkylating agents were available for purchase (and more, of course, could be synthesized). The particular moieties used were chosen to maximize structural dispersion, while limiting the numbers to those conveniently synthesized in the wells of a microtiter plate. In choosing between structurally similar compounds, preference was given to the least substituted compound.

The variable elements included both aliphatic and aromatic groups. Among the aliphatic groups, both acyclic and cyclic (mono- or poly-) structures, substituted or not, were tested. (While all of the acyclic groups were linear, it would have been feasible to introduce a branched aliphatic). The aromatic groups featured either single and multiple rings, fused or not, substituted or not, and with heteroatoms or not. The secondary substitutents included —NH₂, —OH, —OMe, —CN, —Cl, —F, and —COOH. While not used, spacer moieties, such as —O—, —S—, —OO—, —CS—, —NH—, and —NR—, could have been incorporated.

Bunin et al. suggest that instead of using a 1, 4-benzodiazepine as a core structure, one may instead use a 1, 4-benzodiazepine-2,5-dione structure.

As noted by Bunin et al., it is advantageous, although not necessary, to use a linkage strategy which leaves no trace of the linking functionality, as this permits construction of a more diverse library.

Other combinatorial nonoligomeric compound libraries known or suggested in the art have been based on carbamates, mercaptoacylated pyrrolidines, phenolic agents, aminimides, -acylamino ethers (made from amino alcohols, aromatic hydroxy acids, and carboxylic acids), N-alkylamino ethers (made from aromatic hydroxy acids, amino alcohols and aldehydes) 1, 4-piperazines, and 1,4-piperazine-6-ones.

DeWitt, et al., Proc. Nat. Acad. Sci. (USA), 90:6909-13 (1993) describes the simultaneous but separate, synthesis of 40 discrete hydantoins and 40 discrete benzodiazepines. They carry out their synthesis on a solid support (inside a gas dispersion tube), in an array format, as opposed to other conventional simultaneous synthesis techniques (e.g., in a well, or on a pin). The hydantoins were synthesized by first simultaneously deprotecting and then treating each of five amino acid resins with each of eight isocyanates. The benzodiazepines were synthesized by treating each of five deprotected amino acid resins with each of eight 2-amino benzophenone imines.

Chen, et al., J. Am. Chem. Soc., 116:2661-62 (1994) described the preparation of a pilot (9 member) combinatorial library of formate esters. A polymer bead-bound aldehyde preparation was “split” into three aliquots, each reacted with one of three different ylide reagents. The reaction products were combined, and then divided into three new aliquots, each of which was reacted with a different Michael donor. Compound identity was found to be determinable on a single bead basis by gas chromatography/mass spectroscopy analysis.

Holmes, U.S. Pat. No. 5,549,974 (1996) sets forth methodologies for the combinatorial synthesis of libraries of thiazolidinones and metathiazanones. These libraries are made by combination of amines, carbonyl compounds, and thiols under cyclization conditions.

Ellman, U.S. Pat. No. 5,545,568 (1996) describes combinatorial synthesis of benzodiazepines, prostaglandins, beta-turn mimetics, and glycerol-based compounds. See also Ellman, U.S. Pat. No. 5,288,514.

Summerton, U.S. Pat. No. 5,506,337 (1996) discloses methods of preparing a combinatorial library formed predominantly of morpholino subunit structures.

Heterocylic combinatorial libraries are reviewed generally in Nefzi, et al., Chem. Rev., 97:449-472 (1997).

The library is preferably synthesized so that the individual members remain identifiable so that, if a member is shown to be active, it is not necessary to analyze it. Several methods of identification have been proposed, including:

(1) encoding, i.e., the attachment to each member of an identifier moiety which is more readily identified than the member proper. This has the disadvantage that the tag may itself influence the activity of the conjugate.

(2) spatial addressing, e.g., each member is synthesized only at a particular coordinate on or in a matrix, or in a particular chamber. This might be, for example, the location of a particular pin, or a particular well on a microtiter plate, or inside a “tea bag”.

The present invention is not limited to any particular form of identification.

However, it is possible to simply characterize those members of the library which are found to be active, based on the characteristic spectroscopic indicia of the various building blocks.

Solid phase synthesis permits greater control over which derivatives are formed. However, the solid phase could interfere with activity. To overcome this problem, some or all of the molecules of each member could be liberated, after synthesis but before screening.

Examples of candidate simple libraries which might be evaluated include derivatives of the following:

Cyclic Compounds Containing One Hetero Atom

Heteronitrogen
pyrroles
pentasubstituted pyrroles
pyrrolidines
pyrrolines
prolines
indoles
beta-carbolines
pyridines
dihydropyridines
1,4-dihydropyridines
pyrido[2,3-d]pyrimidines
tetrahydro-3H-imidazo[4,5-c] pyridines
Isoquinolines
tetrahydroisoquinolines
quinolones
beta-lactams
azabicyclo[4.3.0]nonen-8-one amino acid
Heterooxygen
furans
tetrahydrofurans
2,5-disubstituted tetrahydrofurans
pyrans
hydroxypyranones
tetrahydroxypyranones
gamma-butyrolactones
Heterosulfur
sulfolenes

Cyclic Compounds with Two or More Hetero atoms

Multiple heteronitrogens
imidazoles
pyrazoles
piperazines
diketopiperazines
arylpiperazines
benzylpiperazines
benzodiazepines
1,4-benzodiazepine-2,5-diones
hydantoins
5-alkoxyhydantoins
dihydropyrimidines
1,3-disubstituted-5,6-dihydopyrimidine-2,4-
diones
cyclic ureas
cyclic thioureas
quinazolines
chiral 3-substituted-quinazoline-2,4-diones
triazoles
1,2,3-triazoles
purines
Heteronitrogen and Heterooxygen
dikelomorpholines
isoxazoles
isoxazolines
Heteronitrogen and Heterosulfur
thiazolidines
N-axylthiazolidines
dihydrothiazoles
2-methylene-2,3-dihydrothiazates
2-aminothiazoles
thiophenes
3-amino thiophenes
4-thiazolidinones
4-melathiazanones
benzisothiazolones
For details on synthesis of libraries, see Nefzi, et al., Chem. Rev., 97: 449-72 (1997), and references cited therein.

Nonbiogenic and Other Mutant Peptides

While the peptides of the combinatorial library screened by the contemplated cell-based assay are expressed by that cell, and hence must be biogenic (composed of the 20 genetically encoded amino acids), once a binding peptide is so identified, one may prepare similar, nonbiogenic peptides and test them for activity.

Amino acids are the basic building blocks with which peptides and proteins are constructed. Amino acids possess both an amino group (—NH₂) and a carboxylic acid group (—COOH). Many amino acids, but not all, have the structure NH₂—CHR—COOH, where R is hydrogen, or any of a variety of functional groups.

Of the genetically encoded AAs, all save Glycine are optically isomeric, however, only the L-form is found in humans. Nevertheless, the D-forms of these amino acids do have biological significance; D-Phe, for example, is a known analgesic.

Many other amino acids are also known, including: 2-Aminoadipic acid; 3-Aminoadipic acid; beta-Aminopropionic acid; 2-Aminobutyric acid; 4-Aminobutyric acid (Piperidinic acid); 6-Aminocaproic acid; 2-Aminoheptanoic acid; 2-Aminoisobutyric acid, 3-Aminoisobutyric acid; 2-Aminopimelic acid; 2,4-Diaminobutyric acid; Desmosine; 2,2′-Diaminopimelic acid; 2,3-Diaminopropionic acid; N-Ethylglycine; N-Ethylasparagine; Hydroxylysine; allo-Hydroxylysine; 3-Hydroxyproline; 4-Hydroxyproline; Isodesmosine; allo-Isoleucine; N-Methylglycine (Sarcosine); N-Methylisoleucine; N-Methylvaline; Norvaline; Norleucine; and Ornithine.

Peptides are constructed by condensation of amino acids and/or smaller peptides. The amino group of one amino acid (or peptide) reacts with the carboxylic acid group of a second amino acid (or peptide) to form a peptide (—NHCO—) bond, releasing one molecule of water. Therefore, when an amino acid is incorporated into a peptide, it should, technically speaking, be referred to as an amino acid residue.

The core of that residue is the moiety which excludes the —NH and —CO linking functionalities which connect it to other residues. This moiety consists of one or more main chain atoms (see below) and the attached side chains.

The main chain moiety of each AA consists of the —NH and —CO linking functionalities and a core main chain moiety. Usually the latter is a single carbon atom. However, the core main chain moiety may include additional carbon atoms, and may also include nitrogen, oxygen or sulfur atoms, which together form a single chain. In a preferred embodiment, the core main chain atoms consist solely of carbon atoms.

The side chains are attached to the core main chain atoms. For alpha amino acids, in which the side chain is attached to the alpha carbon, the C-1, C-2 and N-2 of each residue form the repeating unit of the main chain, the word “side chain” refers to the C-3 and higher numbered carbon atoms and their substituents. It also includes H atoms attached to the main chain atoms.

Amino acids may be classified according to the number of carbon atoms which appear in the main chain in between the carbonyl carbon and amino nitrogen atoms which participate in the peptide bonds. Among the 150 or so amino acids which occur in nature, alpha, beta, gamma and delta amino acids are known. These have 1-4 intermediary carbons. Epsilon amino acids (5 intermediary carbons) are commercially available. Only alpha amino acids occur in proteins. Proline is a special case of an alpha amino acid; its side chain also binds to the peptide bond nitrogen.

For beta and higher order amino acids, there is a choice as to which main chain core carbon a side chain other than H is attached to. The preferred attachment site is the C-2 (alpha) carbon, i.e., the one adjacent to the carboxyl carbon of the —CO linking functionality. It is also possible for more than one main chain atom to carry a side chain other than H. However, in a preferrred embodiment, only one main chain core atom carries a side chain other than H.

A main chain carbon atom may carry either one or two side chains; one is more common. A side chain may be attached to a main chain carbon atom by a single or a double bond; the former is more common.

A peptide is composed of a plurality of amino acid residues joined together by peptidyl (—NHCO—) bonds. A biogenic peptide is a peptide in which the residues are all genetically encoded amino acid residues; it is not necessary that the biogenic peptide actually be produced by gene expression.

The peptides of the present invention include peptides whose sequences are disclosed in this specification, or sequences differing from the above solely by no more than one nonconservative substitution and/or one or more conservative substitutions, preferably no more than a single conservative substitution. The substitutions may be of non-genetically encoded (exotic) amino acids, in which case the resulting peptide is nonbiogenic. Preferably, the peptides are biogenic.

If the peptide is being expressed in a cell, all of its amino acids must be biogenic (unless the cell is engineered to alter certain amino acids post-expression, or the peptide is recovered and modified in vitro). If it is produced nonbiologically (e.g., Merrifield-type synthesis) or by semisynthesis, it may include nonbiogenic amino acids.

Additional peptides within the present invention may be identified by systematic mutagenesis of the lead peptides, e.g.

(a) separate synthesis of all possible single substitution (especially of genetically encoded AAs) mutants of each lead peptide, and/or

(b) simultaneous binomial random alanine-scanning mutagenesis of each lead peptide, so each amino acids position may be either the original amino acid or alanine (alanine being a semi-conservative substitution for all other amino acids), and/or

(c) simultaneous random mutagenesis sampling conservative substitutions of some or all positions of each lead peptide, the number of sequences in total sequences space for a given experiment being such that any sequence, if active, is within detection limits (typically, this means not more than about 10¹⁰ different sequences).

Substitutions are preferably at sites shown to tolerate mutation by the mutagenic strategies set forth above.

The mutants are tested for activity, and, if active, are considered to be within “peptides of the present invention”. Even inactive mutants contribute to our knowledge of structure-activity relationships and thus assist in the design of peptides, peptoids, and peptidomimetics.

The core sequences of the peptides may be identified by systematic truncation, starting at the N-terminal, the C-terminal, or both simultaneously or sequentially. The truncation may be one amino acid at a time, but preferably, to speed up the process, is of 10-50% of the molecule at one time. If a given truncation is unsuccessful, one retreats to a less dramatic truncation intermediate between the last successful truncation and the last unsuccessful truncation.

Most extensions should be tolerated. However, if one is not, it may be helpful to introduce a linker, such as one made primarily of amino acids such as Glycine (introduces flexibility), and Proline (introduce a rigid extension), or other amino acids favored in protein turns, loops and interdomain boundaries. Indeed, the sequences of such segments may be used directly as linkers.

Preferably, substitutions of exotic amino acids for the original amino acids take the form of

(I) replacement of one or more hydrophilic amino acid side chains with another hydrophilic organic radical, not more than twice the volume of the original side chain, or

(II) replacement of one or more hydrophobic amino acid side chains with another hydrophobic organic radical, not more than twice the volume of the original side chain.

The exotic amino acids may be alpha or non-alpha amino acids (e.g., beta alanine). They may be alpha amino acids with 2 R groups on the Ca, which groups may be the same or different. They may be dehydro amino acids (HOOC—C(NH₂)═CHR).

Cyclic Peptides

Many naturally occurring peptide are cyclic. Cyclization is a common mechanism for stabilization of peptide conformation thereby achieving improved association of the peptide with its ligand and hence improved biological activity. Cyclization is usually achieved by intra-chain cystine formation, by formation of peptide bond between side chains or between - and C-terminals.

Peptoid

A peptoid is an analogue of a peptide in which one or more of the peptide bonds are replaced by pseudopeptide bonds, which may be the same or different.

Such pseudopeptide bonds may be:

Carba Ψ(CH₂—CH₂)

Depsi Ψ(CO—O)

Hydroxyethylene Ψ(CHOH—CH₂)

Ketomethylene Ψ(CO—CH₂)

Methylene-ocy CH₂—O—

Reduced CH₂—NH

Thiomethylene CH₂—S—

Thiopeptide CS—NH

N-modified —NRCO—

Retro-Inverso —CO—NH—

A single peptoid molecule may include more than one kind of pseudopeptide bond. It may include normal peptide bonds.

A peptoid library, composed for peptoids related to one or more lead peptides, may be synthesized and screened. A peptoid library may comprise true peptides, too. For the purposes of introducing diversity into a peptoid library, one may vary (1) the side chains attached to the core main chain atoms of the monomers linked by the pseudopeptide bonds, and/or (2) the the side chains (e.g., the —R of an —NRCO—) of the pseudopeptide bonds. Thus, in one embodiment, the monomeric units which are not amino acid residues are of the structure —NR1-CR2-CO—, where at least one of R1 and R2 are not hydrogen. If there is variability in the pseudopeptide bond, this is most conveniently done by using an —NRCO— or other pseudopeptide bond with an R group, and varying the R group. In this event, the R group will usually be any of the side chains characterizing the amino acids of peptides, as previously discussed.

If the R group of the pseudopeptide bond is not variable in the library, it will usually be small, e.g., not more than 10 atoms (e.g., hydroxyl, amino, carboxyl, methyl, ethyl, propyl).

Peptidomimetic

A peptidomimetic is a molecule which mimics the biological activity of a peptide, by substantially duplicating the pharmacologically relevant portion of the conformation of the peptide, but is not a peptide or peptoid as defined above. Preferably the peptidomimetic has a molecular weight of less than 700 daltons.

Designing a peptidomimetic usually proceeds by:

(a) identifying the pharmacophoric groups responsible for the activity;

(b) determining the spatial arrangements of the pharmacophoric groups in the active conformation of the peptide; and

(c) selecting a pharmaceutically acceptable template upon which to mount the pharmacophoric groups in a manner which allows them to retain their spatial arrangement in the active conformation of the peptide.

Step (a) may be carried out by preparing mutants of the active peptide and determining the effect of the mutation on activity. One may also examine the 3D structure of a complex of the peptide and the receptor for evidence of interactions, e.g., the fit of a side chain of the peptide into a cleft of the receptor; potential sites for hydrogen bonding, etc.).

Step (b) generally involves determining the 3D structure of the active peptide, in the complex, by NMR spectroscopy or X-ray diffraction studies. The initial 3D model may be refined by an energy minimization and molecular dynamics simulation.

Step (c) may be carried out by reference to a template database, see Wilson, et al. Tetrahedron, 49:3655-63 (1993). The templates will typically allow the mounting of 2-8 pharmacophores, and have a relatively rigid structure. For the latter reason, aromatic structures, such as benzene, biphenyl, phenanthrene and benzodiazepine, are preferred. For orthogonal protection techniques, see Tuchscherer, et al., Tetrahedron, 17:3559-75 (1993).

For more information on peptoids and peptidomimetics, see U.S. Pat. No. 5,811,392, U.S. Pat. No. 5,811,512, U.S. Pat. No. 5,578,629, U.S. Pat. No. 5,817,879, U.S. Pat. No. 5,817,757, U.S. Pat. No. 5,811,515.

Analogues

Also of interest are analogues of the disclosed peptides, and other compounds with activity of interest.

Analogues may be identified by assigning a hashed bitmap structural fingerprint to the compound, based on its chemical structure, and determining the similarity of that fingerprint to that of each compound in a broad chemical database. The fingerprints are determined by the fingerprinting software commercially distributed for that purpose by Daylight Chemical Information Systems, Inc., according to the software release current as of Jan. 8, 1999. In essence, this algorithm generates a bit pattern for each atom, and for its nearest neighbors, with paths up to 7 bonds long. Each pattern serves as a seed to a pseudorandom number generator, the output of which is a set of bits which is logically ored to the developing fingerprint. The fingerprint may be fixed or variable size.

The database may be SPRESI'95 (InfoChem GmbH), Index Chemicus (ISI), MedChem (Pomona/Biobyte), World Drug Index (Derwent), TSCA93(EPA) May bridge organic chemical catalog (Maybridge), Available Chemicals Directory (MDLIS Inc.), NCI96 (NCI), Asinex catalog of organic compounds (Asinex Ltd.), or IBIOScreen SC and NP (Inter BioScreen Ltd.), or an inhouse database.

A compound is an analogue of a reference compound if it has a daylight fingerprint with a similarity (Tanamoto coefficient) of at least 0.85 to the Daylight fingerprint of the reference compound.

A compound is also an analogue of a reference compound id it may be conceptually derived from the reference compound by isosteric replacements.

Homologues are compounds which differ by an increase or decrease in the number of methylene groups in an alkyl moiety.

Classical isosteres are those which meet Erlenmeyer's definition: “atoms, ions or molecules in which the peripheral layers of electrons can be considered to be identical”. Classical isosteres include

| Monovalents | Bivalents | Trivalents | Tetra | Annular |
|---|---|---|---|---|
| F, OH, NH₂, CH₃ | —O— | —N═ | ═C═ | —CH═CH— |
| | | | ═Si═ | |
| Cl, SH, PH₂ | —S— | —P═ | —N+═ | —S— |
| Br | —Se— | —As— | ═P+═ | —O— |
| i | —Te— | —Sb— | ═As+═ | —NH— |
| | | —CH═ | ═Sb+═ | |

Nonclassical isosteric pairs include —CO— and —SO₂—, —COOH and —SO₃H, —SO₂NH₂ and —PO(OH)NH₂, and —H and —F, —OC(═O)— and C(═O)O—, —OH and —NH₂.

Bioloqical Assays

While a major purpose of the invention is to minimize the need for biological assays, they cannot be altogether avoided. In order to predict the biological activity of a substance, one must know the biological activities of a reasonable number of reference substances.

A biological assay measures or detects a biological response of a biological entity to a substance. The present invention is concerned with responses which are, at least in part, mediated by a receptor.

The biological entity may be a whole organism, an isolated organ or tissue, freshly isolated cells, an immortalized cell line, or a subcellular component (such as a membrane; this term should not be construed as including an isolated receptor) The entity may be, or may be derived from, an organism which occurs in nature, or which is modified in some way. Modifications may be genetic (including radiation and chemical mutants, and genetic engineering) or somatic (e.g., surgical, chemical, etc.). In the case of a multicellular entity, the modifications may affect some or all cells. The entity need not be the target organism, or a derivative thereof, if there is a reasonable correlation between bioassay activity in the assay entity and biological activity in the target organism.

The entity is placed in a particular environment, which may be more or less natural. For example, a culture medium may, but need not, contain serum or serum substitutes, and it may, but need not, include a support matrix of some kind, it may be still, or agitated. It may contain particular biological or chemical agents, or have particular physical parameters (e.g., temperature), that are intended to nourish or challenge the biological entity.

There must also be a detectable biological marker for the response. At the cellular level, the most common markers are cell survival and proliferation, cell behavior (clustering, motility), cell morphology (shape, color), and biochemical activity (overall DNA synthesis, overall protein synthesis, and specific metabolic activities, such as utilization of particular nutrients, e.g., consumption of oxygen, production of CO₂, production of organic acids, uptake or discharge of ions).

The direct signal produced by the biological marker may be transformed by a signal producing system into a different signal which is more-observable, for example, a fluorescent or colorimetric signal.

The entity, environment, marker and signal producing system are chosen to achieve a clinically acceptable level of sensitivity, specificity and accuracy.

Reference substances should be tested in the appropriate assays relevant to the tissue distribution of the targeted receptor. For instance, for the estrogen receptor which is expressed in breast epithelium, liver mesenchymal cells, osteoclasts and uterine epithelium (among others) appropriate assays would include, among others, breast and uterine epithelial cell proliferation, osteoclast apoptosis, and hepatocyte production of lipids such as triglycerides and cholesterol and lipoproteins such as high density lipoproteins and low density lipoproteins.

If one were to utilize the androgen receptor which is expressed in, among others, prostate epithelium, hepatocytes, striated muscle cells, then one would might chose to carry out assays of the reference substance set for, among others, prostate hypertrophy, hyperplasia or prostate epithelial cell proliferation, muscle cell hyperplasia or hypertrophy and heptotoxicity etc.

As another example, if one were to utilize the beta-2-adrenergic receptor, which is expressed in, among others, the heart, brain and peripheral vasculature, then one may chose to test reference substances in cardiac function assays (such as cardiac rate and eletrocardiographic changes), assays for their impact on blood pressure and assays to evaluate their impact on neuronal activity within the central nervous system.

General Uses

In addition to use as Biokeys, the oligomers identified by the screening assays of the present invention, as binding molecules, may also be used as pharmaceuticals or in diagnostic reagents as described below.

Pharmaceutical Methods and Preparations

The preferred animal subject of the present invention is a mammal. By the term “mammal” is meant an individual belonging to the class Mammalia. The invention is particularly useful in the treatment of human subjects, although it is intended for veterinary uses as well. Preferred nonhuman subjects are of the orders Primata (e.g., apes and monkeys), Artiodactyla or Perissodactyla (e.g., cows, pigs, sheep, horses, goats), Carnivora (e.g., cats, dogs), Rodenta (e.g., rats, mice, guinea pigs, hamsters), Lagomorpha (e.g., rabbits) or other pet, farm or laboratory mammals.

The term “protection”, as used herein, is intended to include “prevention,” “suppression” and “treatment.” “Prevention” involves administration of the protein prior to the induction of the disease (or other adverse clinical condition). “Suppression” involves administration of the composition prior to the clinical apoearance of the disease. “Treatment” involves administration of the protective composition after the appearance of the disease. Protection, including prevention, need not be absolute.

It will be understood that in human and veterinary medicine, it is not always possible to distinguish between “preventing” and “suppressing” since the ultimate inductive event or events may be unknown, latent, or the patient is not ascertained until well after the occurrence of the event or events. Therefore, it is common to use the term “prophylaxis” as distinct from “treatment” to encompass both “preventing” and “suppressing” as defined herein. The term “protection,” as used herein, is meant to include “prophylaxis.” It should also be understood that to be useful, the protection provided need not be absolute, provided that it is sufficient to carry clinical value. An agent which provides protection to a lesser degree than do competitive agents may still be of value if the other agents are ineffective for a particular individual, if it can be used in combination with other agents to enhance the level of protection, or if it is safer than competitive agents. The drug may provide a curative effect, an ameliorative effect, or both.

At least one of the drugs of the present invention may be administered, by any means that achieve their intended purpose, to protect a subject against a disease or other adverse condition. The form of administration may be systemic or topical. For example, administration of such a composition may be by various parenteral routes such as subcutaneous, intravenous, intradermal, intramuscular, intraperitoneal, intranasal, transdermal, or buccal routes. Alternatively, or concurrently, administration may be by the oral route. Parenteral administration can be by bolus injection or by gradual perfusion over time.

A typical regimen comprises administration of an effective amount of the drug, administered over a period ranging from a single dose, to dosing over a period of hours, days, weeks, months, or years.

It is understood that the suitable dosage of a drug of the present invention will be dependent upon the age, sex, health, and weight of the recipient, kind of concurrent treatment, if any, frequency of treatment, and the nature of the effect desired. However, the most preferred dosage can be tailored to the individual subject, as is understood and determinable by one of skill in the art, without undue experimentation. This will typically involve adjustment of a standard dose, e.g., reduction of the dose if the patient has a low body weight.

Prior to use in humans, a drug will first be evaluated for safety and efficacy in laboratory animals. In human clinical studies, one would begin with a dose expected to be safe in humans, based on the preclinical data for the drug in question, and on customary doses for analogous drugs (if any). If this dose is effective, the dosage may be decreased, to determine the minimum effective dose, if desired. If this dose is ineffective, it will be cautiously increased, with the patients monitored for signs of side effects. See, e.g., Berkow et al, eds., The Merck Manual, 15th edition, Merck and Co., Rahway, N.J., 1987; Goodman et al., eds., Goodman and Gilman's The Pharmacological Basis of Therapeutics, 8th edition, Pergamon Press, Inc., Elmsford, N.Y., (1990); Avery's Drug Treatment: Principles and Practice of Clinical Pharmacology and Therapeutics, 3rd edition, ADIS Press, LTD., Williams and Wilkins, Baltimore, Md. (1987), Ebadi, Pharmacology, Little, Brown and Co., Boston, (1985), which references and references cited therein, are entirely incorporated herein by reference.

The total dose required for each treatment may be administered by multiple doses or in a single dose. The protein may be administered alone or in conjunction with other therapeutics directed to the disease or directed to other symptoms thereof.

The appropriate dosage form will depend on the disease, the protein, and the mode of administration; possibilities include tablets, capsules, lozenges, dental pastes, suppositories, inhalants, solutions, ointments and parenteral depots. See, e.g., Berker, supra, Goodman, supra, Avery, supra and Ebadi, supra, which are entirely incorporated herein by reference, including all references cited therein.

In the case of peptide drugs, the drug may be administered in the form of an expression vector comprising a nucleic acid encoding the peptide, such a vector, after in corporation into the genetic complement of a cell of the patient, directs synthesis of the peptide. Suitable vectors include genetically engineered poxviruses (vaccinia), adenoviruses, adeno-associated viruses, herpesviruses and lentiviruses which are or have been rendered nonpathogenic.

In addition to at least one drug as described herein, a pharmaceutical composition may contain suitable pharmaceutically acceptable carriers, such as excipients, carriers and/or auxiliaries which facilitate processing of the active compounds into preparations which can be used pharmaceutically. See, e.g., Berker, supra, Goodman, supra, Avery, supra and Ebadi, supra, which are entirely incorporated herein by reference, included all references cited therein.

Diagnostic Assays

While preliminary screening assays are used to determined the activity of a compound of uncertain activity, diagnostic assays employ a binding molecule of known binding activity, or a conjugate or derivative thereof, as a diagnostic reagent.

For the purpose of the discussion of diagnostic methods and agents which follows, the “binding molecule” may be a peptide, peptoid, peptidomimetic or other analogue of the present invention, or an oligonucleotide of the present invention, which binds the analyte or a binding partner of the analyte. The analyte is a target protein.

In Vitro Assay Methods and Reagents

In vitro assays may be diagnostic assays (using a known binding molecule to detect or measure an analyte) or screening assays (determining whether a potential binding molecule in fact binds a target). The format of these two types of assays is very similar and, while the description below refers to 112 diagnostic assays for analytes, it applies, mutatis mutandis, to the screening of molecules for binding to targets. The in vitro assays of the present invention may be applied to any suitable analyte-containing sample, and may be qualitative or quantitative in nature.

In order to detect the presence, or measure the amount, of an analyte, the assay must provide for a signal producing system (SPS) in which there is a detectable difference in the signal produced, depending on whether the analyte is present or absent (or, in a quantitative assay, on the amount of the analyte). This signal is, or is derived from, one or more observable raw signals.

The raw signal for a particular state (e.g., presence or amount of analyte) is the level of an observable parameter, or of a function dependent on the level(s) of one or more observable parameters. The signal is a difference in raw signals, depending on the states to be differentiated by the assay.

The signal may be direct (increased if the amount of analyte increases) or inverse (decreased raw signal if the amount of analyte increases). The signal may be absolute (in one state, there is no detectable raw signal at all) or relative (a change in the level of the raw signal, or of the rate of change in the level of the raw signal). The signal may be discrete (yes or no, depending on the level of the raw signal relative to some threshold) or continuous in value. The signal may be simple (based on a single raw signal) or composite (based on a plurality of raw signals).

The detectable raw signal may be one which is visually detectable, or one detectable only with instruments. Possible raw signals include production of colored or luminescent products, alteration of the characteristics (including amplitude or polarization) of absorption or emission of radiation by an assay component or product, and precipitation or agglutination of a component or product. The raw signal may be monitored manually or automatically.

The component of the signal producing system which is most intimately associated with the diagnostic reagent is called the “label”. A label may be, e.g., a radioisotope, a fluorophore, an enzyme, a co-enzyme, an enzyme substrate, an electron-dense compound, or an agglutinable particle. One diagnostic reagent is a conjugate, direct or indirect, or covalent or noncovalent, of a label with a binding molecule of the invention.

The radioactive isotope can be detected by such means as the use of a gamma counter or a scintillation counter or by autoradiography. Isotopes which are particularly useful for the purpose of the present invention are ³H, ¹²⁵I, ¹³¹I, ³⁵S, ¹⁴C, and, preferably, ¹²⁵I.

It is also possible to label a compound with a fluorescent compound. When the fluorescently labeled antibody is exposed to light of the proper wave length, its presence can then be detected due to fluorescence. Among the most commonly used fluorescent labelling compounds are fluorescein isothiocyanate, rhodamine, phycoerythrin, phycocyanin, allophycocyanin, o-phthaldehyde and fluorescamine.

Alternatively, fluorescence-emitting metals such as ¹²⁵Eu, or others of the lanthanide series, may be attached to the binding protein using such metal chelating groups as diethylenetriaminepentaacetic acid (DTPA) of ethylenediamine-tetraacetic acid (EDTA).

The binding molecules also can be detectably labeled by coupling to a chemiluminescent compound. The presence of the chemiluminescent compound is then determined by detecting the presence of luminescence that arises during the course of a chemical reaction after a suitable reactant is provided. Examples of particularly useful chemiluminescent labeling compounds are luminol, isolumino, theromatic acridinium ester, imidazole, acridinium salt and oxalate ester.

Likewise, a bioluminescent compound may be used to label the binding molecule. Bioluminescence is a type of chemiluminescence found in biological systems in which a catalytic protein increases the efficiency of the chemiluminescent reaction. The presence of a bioluminescent protein is determined by detecting the presence of luminescence. Important bioluminescent compounds for purposes of labeling are luciferin, luciferase and aequorin.

Enzyme labels, such as horseradish peroxidase and alkaline phosphatase, are preferred. When an enzyme label is used, the signal producing system must also include a substrate for the enzyme. If the enzymatic reaction product is not itself detectable, the SPS will include one or more additional reactants so that a detectable product appears.

Assays may be divided into two basic types, heterogeneous and homogeneous. In heterogeneous assays, the interaction between the affinity molecule and the analyte does not affect the label, hence, to determine the amount or presence of analyte, bound label must be separated from free label. In homogeneous assays, the interaction does affect the activity of the label, and therefore analyte levels can be deduced without the need for a separation step.

In general, a target-binding molecule of the present invention may be used diagnostically in the same way that a target-binding antibody is used. Thus, depending on the assay format, it may be used to assay the target, or by competitive inhibition, other substances which bind the target. The sample will normally be a biological fluid, such as blood, urine, lymph, semen, milk, or cerebrospinal fluid, or a fraction or derivative thereof, or a biological tissue, in the form of, e.g., a tissue section or homogenate. However, the sample conceivably could be (or derived from) a food or beverage, a pharmaceutical or diagnostic composition, soil, or surface or ground water. If a biological fluid or tissue, it may be taken from a human or other mammal, vertebrate or animal, or from a plant. The preferred sample is blood, or a fraction or derivative thereof.

In one embodiment, the binding molecule is insolubilized by coupling it to a macromolecular support, and target in the sample is allowed to compete with a known quantity of a labeled or specifically labelable target analogue. (The conjugate of the binding molecule to a macromolecular support is another diagnostic agent within the present invention.) The “target analogue” is a molecule capable of competing with target for binding to the binding molecule, and the term is intended to include target itself. It may be labeled already, or it may be labeled subsequently by specifically binding the label to a moiety differentiating the target analogue from authentic target. The solid and liquid phases are separated, and the labeled target analogue in one phase is quantified. The higher the level of target analogue in the solid phase, i.e., sticking to the binding molecule, the lower the level of target analyte in the sample.

In a “sandwich assay”, both an insolubilized target-binding molecule, and a labeled target-binding molecule are employed. The target analyte is captured by the insolubilized target-binding molecule and is tagged by the labeled target-binding molecule, forming a tertiary complex. The reagents may be added to the sample in either order, or simultaneously. The target-binding molecules may be the same or different, and only one need be a target-binding molecule according to the present invention (the other may be, e.g., an antibody or a specific binding fragment thereof). The amount of labeled target-binding molecule in the tertiary complex is directly proportional to the amount of target analyte in the sample.

The two embodiments described above are both heterogeneous assays. However, homogeneous assays are conceivable. The key is that the label be affected by whether or not the complex is formed.

A label may be conjugated, directly or indirectly (e.g., through a labeled anti-target-binding molecule antibody), covalently (e.g., with SPDP) or noncovalently, to the target-binding molecule, to produce a diagnostic reagent. Similarly, the target binding molecule may be conjugated to a solid-phase support to form a solid phase (“capture”) diagnostic reagent. Suitable supports include glass, polystyrene, polypropylene, polyethylene, dextran, nylon, amylases, natural and modified celluloses, polyacrylamides, agaroses, and magnetite. The nature of the carrier can be either soluble to some extent or insoluble for the purposes of the present invention. The support material may have virtually any possible structural configuration so long as the coupled molecule is capable of binding to its target. Thus the support configuration may be spherical, as in a bead, or cylindrical, as in the inside surface of a test tube, or the external surface of a rod. Alternatively, the surface may be flat such as a sheet, test strip, etc.

In Vivo Diagnostic Uses

Analyte-binding molecules can be used for in vivo imaging.

Radio-labelled binding molecule may be administered to the human or animal subject. Administration is typically by injection, e.g., intravenous or arterial or other means of administration in a quantity sufficient to permit subsequent dynamic and/or static imaging using suitable radio-detecting devices. The preferred dosage is the smallest amount capable of providing a diagnostically effective image, and may be determined by means conventional in the art, using known radio-imaging agents as a guide.

Typically, the imaging is carried out on the whole body of the subject, or on that portion of the body or organ relevant to the condition or disease under study. The radio-labelled binding molecule has accumulated. The amount of radio-labelled binding molecule accumulated at a given point in time in relevant target organs can then be quantified.

A particularly suitable radio-detecting device is a scintillation camera, such as a gamma camera. A scintillation camera is a stationary device that can be used to image distribution of radio-labelled binding molecule. The detection device in the camera senses the radioactive decay, the distribution of which can be recorded. Data produced by the imaging system can be digitized. The digitized information can be analyzed over time discontinuously or continuously. The digitized data can be processed to produce images, called frames, of the pattern of uptake of the radio-labelled binding protein in the target organ at a discrete point in time. In most continuous (dynamic) studies, quantitative data is obtained by observing changes in distributions of radioactive decay in target organs over time. In other words, a time-activity analysis of the data will illustrate uptake through clearance of the radio-labelled binding molecule by the target organs with time.

Various factors should be taken into consideration in selecting an appropriate radioisotope. The radioisotope must be selected with a view to obtaining good quality resolution upon imaging, should be safe for diagnostic use in humans and animals, and should preferably have a short physical half-life so as to decrease the amount of radiation received by the body. The radioisotope used should preferably be pharmacologically inert, and, in the quantities administered, should not have any substantial physiological effect.

The binding molecule may be radio-labelled with different isotopes of iodine, for example ₁₂₃I, ¹²⁵I, or ¹³¹I (see for example, U.S. Pat. No. 4,609,725). The extent of radio-labeling must, however be monitored, since it will affect the calculations made based on the imaging results (i.e. a diiodinated binding molecule will result in twice the radiation count of a similar monoiodinated binding molecule over the same time frame).

In applications to human subjects, it may be desirable to use radioisotopes other than ¹²⁵I for labelling in order to decrease the total dosimetry exposure of the human body and to optimize the detectability of the labelled molecule (though this radioisotope can be used if circumstances require). Ready availability for clinical use is also a factor. Accordingly, for human applications, preferred radio-labels are for example, ^{99m} Tc, ⁶⁷Ga, ⁶⁸Ga, ⁹⁰y, ¹¹¹In, ^{113m}In, ¹²³I, ¹⁸⁶Re, ¹⁸⁸Re or ²¹¹At.

The radio-labelled binding molecule may be prepared by various methods. These include radio-halogenation by the chloramine—T method or the lactoperoxidase method and subsequent purification by HPLC (high pressure liquid chromatography), for example as described by J. Gutkowska et al in “Endocrinology and Metabolism Clinics of America: (1987) 16 (1):183. Other known method of radio-labelling can be used, such as IODOBEADS™.

There are a number of different methods of delivering the radio-labelled binding molecule to the end-user. It may be administered by any means that enables the active agent to reach the agent's site of action in the body of a mammal. If the molecule is digestible when administered orally, parenteral administration, e.g., intravenous, subcutaneous, or intramuscular, would ordinarily be used to optimize absorption.

Other Uses

The binding molecules of the present invention may also be used to purify target from a fluid, e.g., blood. For this purpose, the target-binding molecule is preferably immobilized on a solid-phase support. Such supports include those already mentioned as useful in preparing solid phase diagnostic reagents.

Peptides, in general, can be used as molecular weight markers for reference in the separation or purification of peptides by electrophoresis or chromatography. In many instances, peptides may need to be denatured to serve as molecular weight markers. A second general utility for peptides is the use of hydrolyzed peptides as a nutrient source. Hydrolyzed peptide are commonly used as a growth media component for culturing microorganisms, as well as a food ingredient for human consumption. Enzymatic or acid hydrolysis is normally carried out either to completion, resulting in free amino acids, or partially, to generate both peptides and amino acids. However, unlike acid hydrolysis, enzymatic hydrolysis (proteolysis) does not remove non-amino acid functional groups that may be present. Peptides may also be used to increase the viscosity of a solution.

The peptides of the present invention may be used for any of the foregoing purposes, as well as for therapeutic and diagnostic purposes as discussed further earlier in this specification.

### EXAMPLES

Intracellular Screening of Peptide Libraries for Peptides Whose Binding Mediates Activation of Cellular Receptors

In these Examples, we show that it is feasible to use a cell-based assay to screen for peptides which bind receptors. Moreover, in these assay examples, the receptor is liganded, and hence the Example shows that it is feasible to use a cell-based assay to screen for potential BioKeys. We call this intracellular screening because the receptor is, at the time of binding, inside a cell, a more natural context than free in solution or immobilized on a nonliving support.

### Example 501

### Estrogen Receptor

A directed peptide library (X₅LXXLLX₅, SEQ ID NO:264) containing the LXXLL motif (previously identified as common in peptides binding estrogen receptor) as a carboxy-terminal fusion to the Gal4 DNA binding domain was constructed with an overall complexity of ˜1×10⁷ independent peptides. The LXXLL motif library peptides were encoded by the degenerate DNA sequence (NNK)₅TTA(NNK)₂(TTA)₂(NNK)₅ (SEQ ID NO:265); TTA is the preferred Leu codon in yeast. NNK encodes a stop codon so a few of the peptides generated will be shorter than 15 a.a. The shortest interacting peptides will likely be 10-mers. This library was transformed into the Saccharomyces cerevisiae PJ69α yeast strain expressing estrogen receptor (ER) α as a carboxy-terminal fusion to the Gal4-activation domain. Following transformation with the plasmid library, interaction between peptides and ER α were selected using media containing 100 nM estradiol and 25 mM 3-aminotriazole but lacking leucine, tryptophan, and histidine. Those colonies able to grow by inducing the integrated GAL4 driven HIS3 reporter were transferred into 0.25 ml of rich media in a 0.5 ml block. 0.25 μl of this suspension was plated onto the same selective media as above, with or without 100 nM estradiol.

The colonies that displayed estradiol-dependent growth on media lacking histidine were subjected to a second screen using β-galactosidase activity to confirm ligand dependence. Dilutions (1-10) of cell suspensions were made into 200 μl rich media with and without 500 nM estradiol in 96-well plates for growth overnight at 30 degrees. Cells were pelleted by centrifugation, the media removed and the cells lysed with buffer containing 2.5% CHAPS (3-(3-Cholamidopropyl) dimethyl ammonio-1-propanesulfonate) detergent. A preliminary OD₆₀₀ was determined to normalize β-galactosidase activity of each well with cell density. Buffer containing substrate (chlorophenol red-β-galactopyranoside, CPRG, Boehringer Mannheim) was added and the reaction monitored by the increase in OD₅₉₅ due to product development. β-galactosidase activity for each well was normalized to the initial OD₆₀₀ and the change induced by ligand determined using the normalized β-galactosidase activity in the presence and absence of 500 nM estradiol. Yeast clones that generated a value equal to or greater than 0.5 were verified using the same method. Verified clones were then subjected to a final test of ligand induced β-galactosidase activity. Yeast protein extracts were prepared and activity assessed using equal amounts of protein from cultures with and without estradiol (FIG. 1). Ten clones displaying the greatest ligand induced β-galactosidase activity were studied further.

Plasmids containing the peptides were isolated from the yeast cells and the amino acid sequences (Table 501) deduced by DNA sequencing of the library inserts. In Table 501, the underlined sequence is encoded by vector DNA, and the “*” represents the endogenous stop codon of the vector, and is downstream of the cloning site. Several peptides contain the vector-encoded LDLQPS (SEQ ID NO:266) sequence. B5H10 is believed to be the result of a double insert, with the second part being encoded by the complementary sequence. Peptides B1A1 and B1G8 were shorter than the other isolates because the clones encoding them carry a premature stop codon, which is inherent in the NNK library. Because of a frame shift, neither peptide B6A1 nor B3E2 contained the sequence encoded by vector DNA.

The newly isolated peptides had a high level of similarity to those isolated previously using phage display.

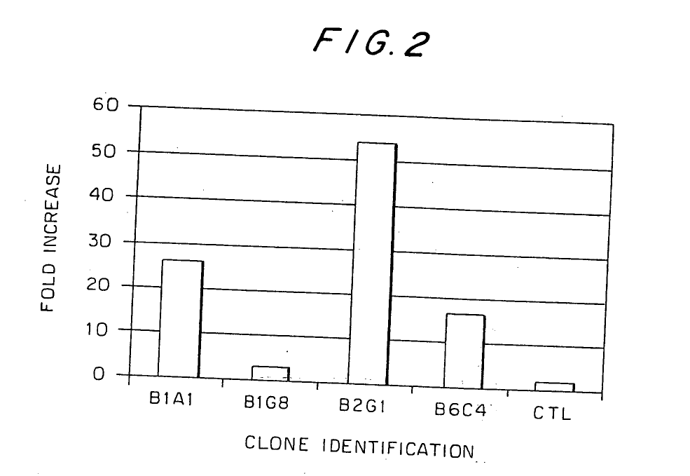

These peptide library member sequences were further tested in a modified mammalian two-hybrid system for their ability to interact specifically with ERα in Huh-7 human hepatoma cells. The term “modified” is used because (1) the system is dependent on ligand, and (2) a nuclear receptor-activation domain construct (of yeast Gal4) is used instead of the more usual (library peptide)-(AD) construct. Isolated library plasmid inserts were PCR amplified using primers with convenient restriction sites to allow subcloning of the products into the yeast Gal4 DBD mammalian two-hybrid vector (pM, Clonetech) (cloning procedure). FIG. 2 shows that these peptides interact with ER α to a similar or greater extent then in the yeast system and confirms the suitability of the use of this peptide identification method. Our success in finding specific ER peptides using the yeast two-hybrid system suggests that it will be possible to identify peptides which bind other receptors utilizing this same system, or other yeast or mammalian two-hybrid assay systems.

It is noteworthy that this system was successful even though nuclear receptors contain their own DNA-binding and activation domains, and therefore the possibility of interference with the exogenous DBD and AD existed.

### Example 502

### Androgen Receptor

We generated unbiased libraries of peptides (X₁₅) fused to the Gal4 DBD for expression in yeast. Our library contained predominantly 15 random amino acid residues/peptide in order to identify motifs that might interact with nuclear receptor domains. If ligand is provided to the cells, one can screen for peptides that interact with the ligand-bound receptor. Some of these peptides will be ligand-specific in their binding activity. The random library was made using synthetic oligonucleotides of the sequence (NNK)₁₅ (SEQ ID NO:267), where K=G/T. NNK encodes one stop codon; hence, a few peptides will be shorter than 15 a.a.; peptides as short as 5 a.a. may have binding activity. Included at the ends of the oligonucleotide were the synthetic restriction endonuclease cleavage sites, EcoRI or Mfe I (5′ end) and Sal I (3′ end). Oligonucleotides were annealed, then cleaved with excess restriction endonuclease and purified on agarose gels. The purified set of oligonucleotides was subcloned as an in-frame carboxy-terminal fusion to a Gal4 DNA binding domain (plasmid vector pMA424 derivative). The library plasmid bank was transformed into a strain of yeast [Saccharomyces cerevisiae PJ69-4α MATα trp1-901 leu2-3, 112 ura3-52 his3-200 gal4Δ gal80Δ LYS2::GAL1-HIS3 GAL2-ADE2 met2::GAL7-lacZ] containing a Gal4 DNA binding element upstream of an integrated HIS3 (Sequence deposit GI:3780 imidazole glycerol phosphate dehydratase CAA 27003) and plasmid expressing the Gal4 transcriptional activation domain (AD) fused in frame with AR (in plasmid vector pGAD-C2, sequence deposit GI:1595843 U70025) by the lithium acetate method. The transformation mix was divided and grown on plates of selective media containing ligand and 25 mM 3-aminotriazole at 30° C. until colonies appeared in 3-7 days. The selective media contained dextrose as the sugar source and lacked the amino acids tryptophan, histidine and leucine to ensure maintenance of the peptide library and receptor fusion plasmids (TRP1 and LEU2 gene products encoded on the plasmids) and requiring Gal4-HIS3 reporter activity. Dihydrotestosterone (DHT) and medroxyprogesterone (MPA) were added to the media at 100 nM to identify peptides that interact with the receptor in the presence of ligand. After colonies appeared they were picked and dispersed into individual wells of a 96-well plate containing rich media (YPD).

To determine ligand dependence, two microliter aliquots were removed from the cell suspension, and arrayed and spotted onto each of two rectangular agar plates, one with 100 nM ligand and one without ligand. Growth was monitored daily.

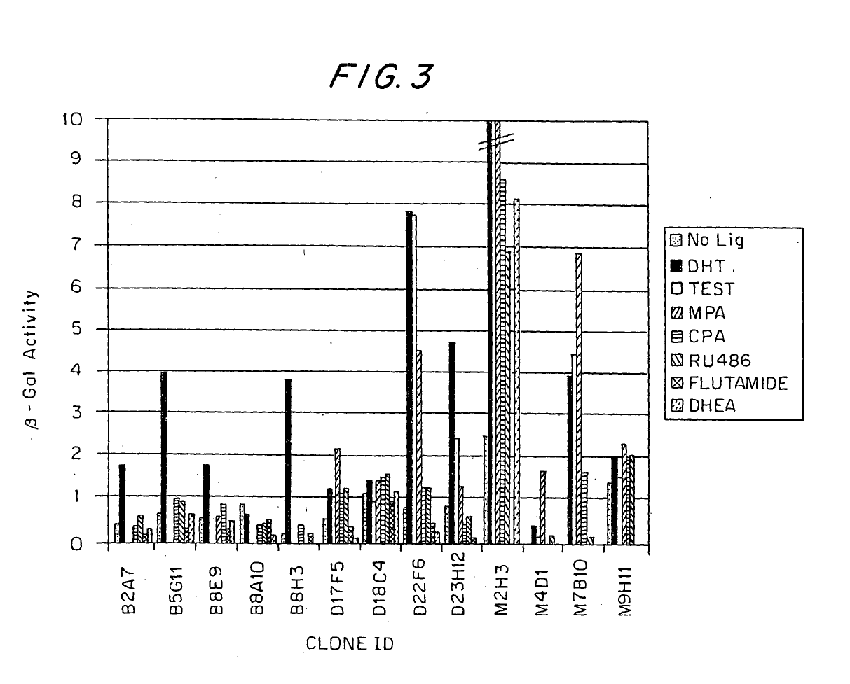

A secondary test of ligand dependent activation was performed using the integrated GAL4-driven lacZ gene. Cell suspensions of the initial positive clones were diluted 20-fold into 20 microliters of rich media in a microplate and grown overnight at 30° C. in the presence and absence of ligand. Microplates were centrifuged for 5 minutes at 3000 rpm in a swinging table top centrifuge to pellet the yeast cells, and the overlying media was aspirated. Cells were then lysed with 10 microliters of buffer containing 2.5% CHAPS. An OD₆₅₀ density determination was made to normalize the β-galactosidase activity. β-galactosidase activity was monitored following the addition of buffer containing chlorophenol red-1-galactopyranoside (CPRG) (Boehringer Mannheim) substrate at a final concentration of 0.5 mM. Using a Vmax kinetic microplate reader, the difference in OD575 (or OD595) and OD650 was recorded during the 10 minute duration of the experiment. The maximum slope value of this measurement was normalized to cell density from the initial OD650 value, and the relative ligand dependence of β-galactosidase was determined by comparing the values plus and minus ligand. As shown in FIG. 3, the clones which are verified using the microplate assay were then tested for ligand induced β-galactosidase activity (as in FIG. 1) using standard liquid based assays of protein extracts.

Known androgen receptor (AR) ligands include the following:

| | |
|---|---|
| DHT: | dihydrotestosterone |
| Test: | testosterone |
| MPA: | medroxyprogesterone acetate |
| CPA, CYP: | cyproterone acetate |
| RU486: | mifepristone |
| DHEA: | dehydroepiandosterone |
| FLUT: | flutamide |

In FIG. 3, all of these ligands were tested. The plasmids from true positives were extracted using standard yeast extraction protocols and re-transformed to verify the positive phenotype. Peptides from the true positives were deduced by DNA sequence analysis of the rescued plasmid or a PCR product derived from the plasmid (see Tables 502A and 502B).

We have used known AR agonists (for example, testosterone, dihydrotestosterone, mibolerone) and known antagonists (for example, cyproterone acetate, flutamide) to determine the specificities of the peptides isolated using either phage display or the modified two-hybrid yeast expression system. (D30/1269-peptide isolated by phage display, all others by present in vivo selection.) Previously, it was found to be possible to identify conformation-specific Estrogen receptor a binding peptides, screened through phage display, using just three forms of the estrogen receptor: non-liganded, estradiol-bound and 4-hydroxytamoxifen-bound. Isolation of AR peptides using a similar number of receptor conformation should be sufficient to isolate conformation specific probes to modulated AR.

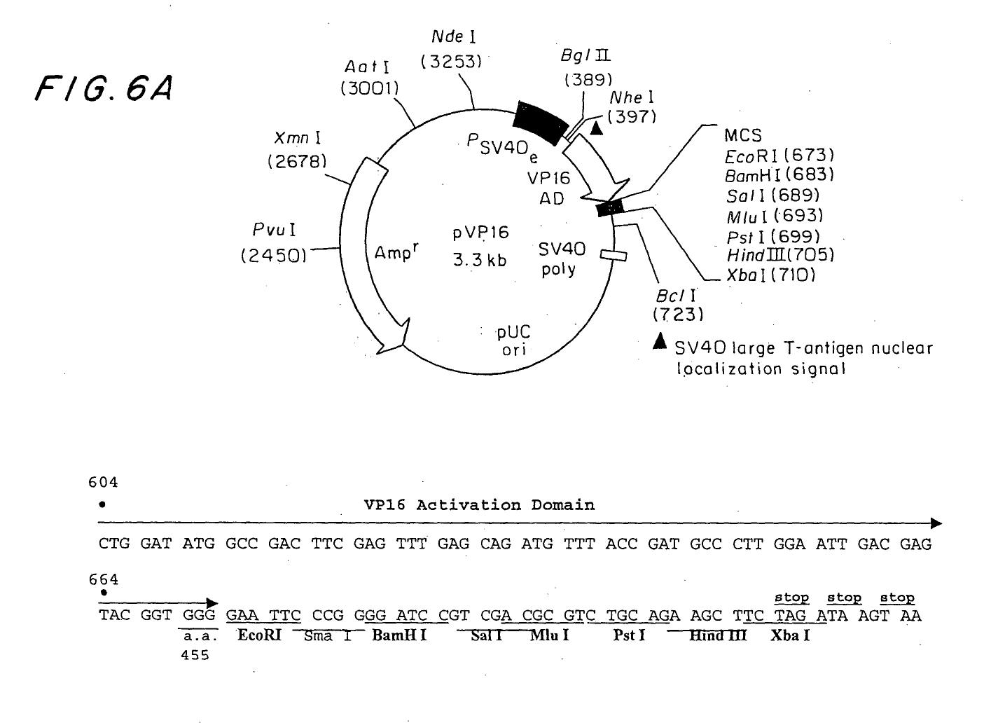

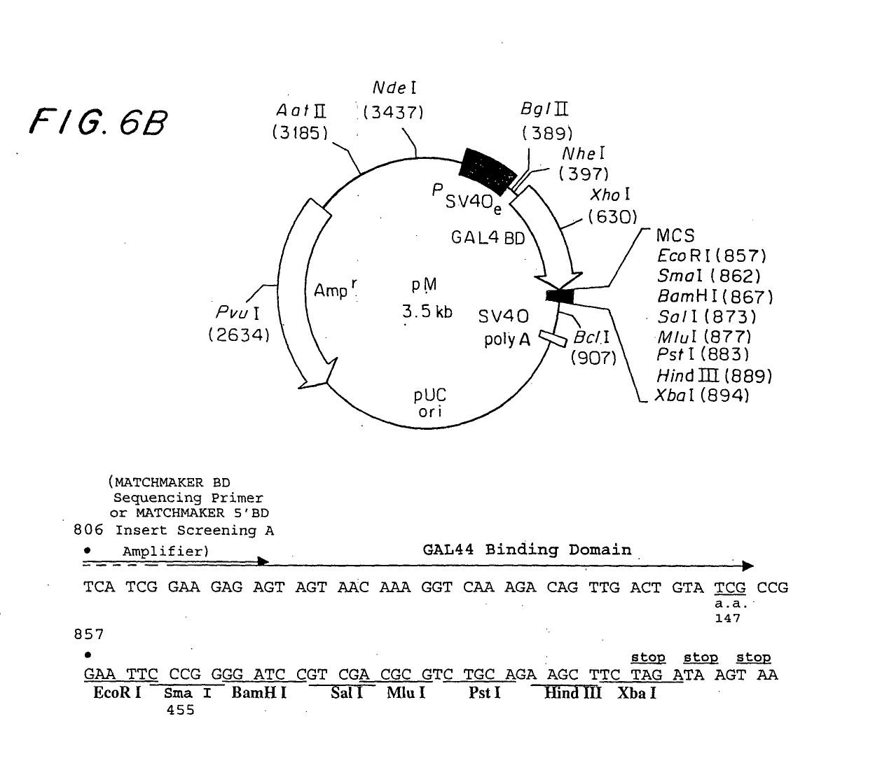

We used a mammalian two-hybrid system to determine specificities of the AR-binding peptides previously identified, through screening of the aforementioned yeast library, as ligand-dependent in their binding activity. Ligand was provided to the cells so the interaction could be observed. The human hepatoma cell line, Huh-7, was used as the recipient of two-hybrid vectors, pM and pVP16 (Clontech) (FIGS. 6A and 6B), containing fusions with peptide and AR, respectively. According to Clontech literature, the Clontech vectors pM and VP16 generate fusions of protein X with the GAL4 DNA-BD and fusions of protein Y with the VP16AD, in the Mammalian MM Two-Hybrid Assay Kit (#K1602-1). (Note: pM and VP16 contain unique cloning sites in the same order and reading frame as the DNA-BD and AD, respectively, vectors in the yeast MATCHMAKER Two-Hybrid Systems.) A third vector, pG5CAT, provides a CAT reporter gene under the control of a GAL4-responsive element and minimal promoter of the adenovirus E1b. The three vectors are cotransfected into any suitable mammalian host cell line by standard methods. The interaction between proteins X and Y is then assayed by measuring CAT gene expression by any standard method. In the absence-of activation (i.e., no protein-protein interaction), the minimal E1b promoter will not express significant levels of CAT.

Unique cloning sites (pM): EcoR1, Sma 1, BamH, Sal 1, Mlu 1, Pst 1, Hind III, Xba 1. Unique cloning site (pVP16): EcoR 1, BamH, Sal 1, Mlu 1, Pst 1, Hind III, Xba 1.

The complete sequence of cloning vector pVP16 is deposited as NCBI U89963. The complete sequence of cloning vector pM is deposited as NCBI U89962.

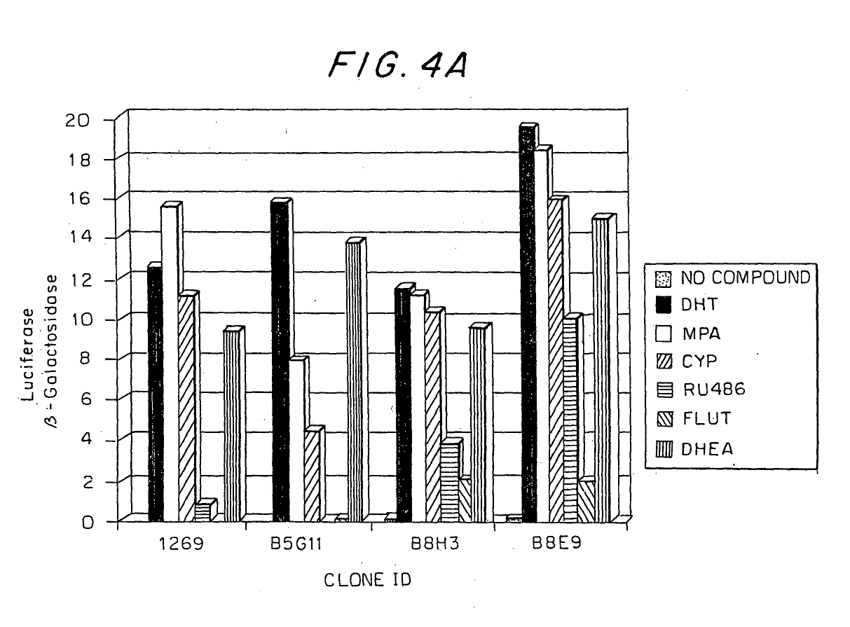

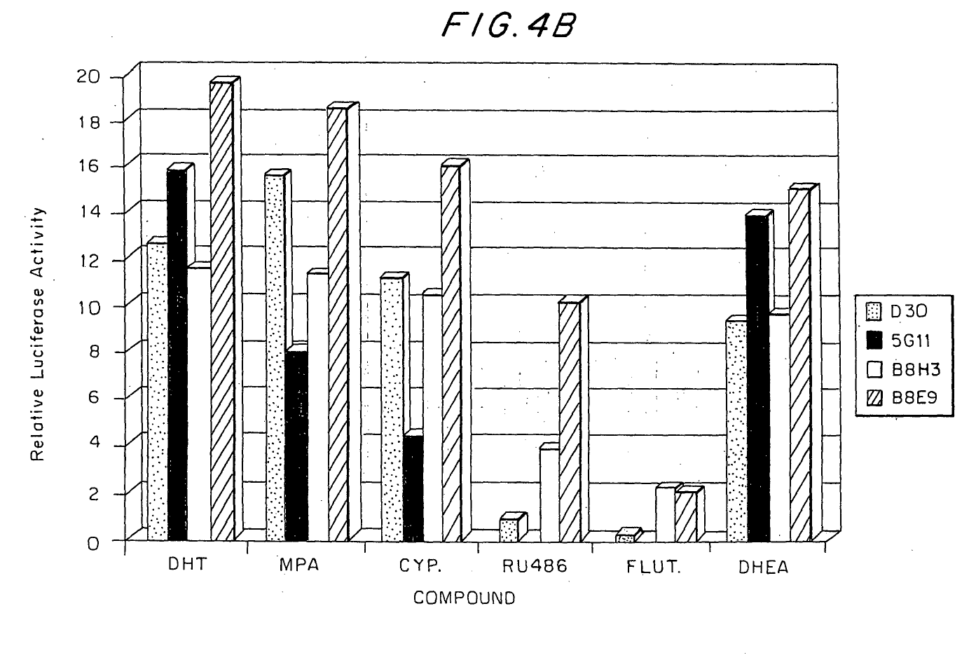

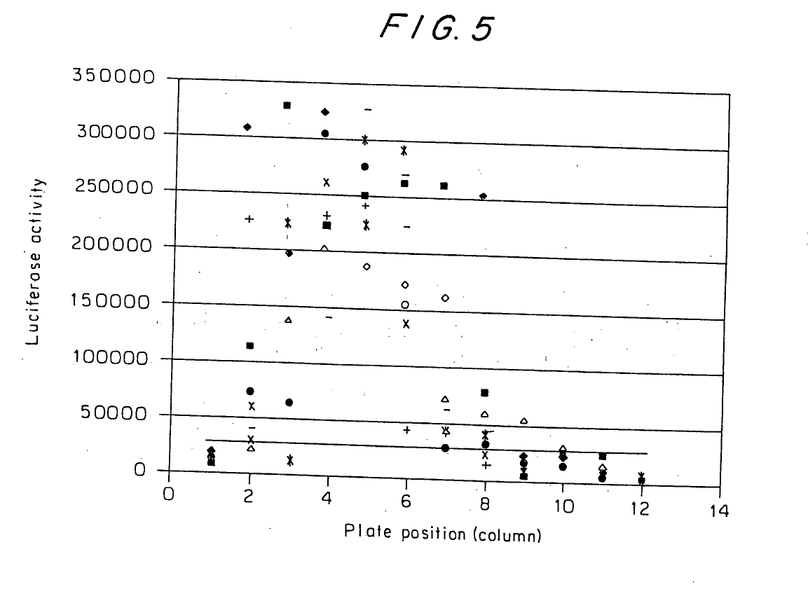

Briefly, cells are seeded in a 24-well culture plate to a density of 50-80% confluence in phenol red-free media containing charcoal-dextran stripped fetal bovine serum (10%). Following incubation overnight, cells are transfected with plasmids pM-peptide, pVP16-AR, pCMV-b-gal, and p5XGAL-Luc3 DNAs using lipofectamine 2000 (LifeTech) according to the manufacturer's protocol. Transfections are allowed to proceed for 6 hours and the transfection media is aspirated and replaced with recovery media for 18 hours. Following the recovery period, cells are treated with compound for 24 hours prior to cell harvesting. Cells are washed with PBS and then lysed using the lysis buffer from the Galacto-Light Plus β-galactosidase assay kit (Tropix). Assays for luciferase and β-galactosidase were performed as described by the manufacturer. Peptide/AR interactions are described in terms of the luciferase activity normalized for transfection efficiency using β-galactosidase activity (see FIG. 4A). Two types of useful information arose from these methods: 1) we generated a panel of useful peptide probes to analyze the conformational state of AR, and 2) we “fingerprinted” AR modulators with respect to AR conformation (FIG. 4B). FIG. 4A compares the ability of peptides 1269, B5G11, B8H3, and B9E9 to interact with the androgen receptor in each of seven different receptor conformations (ligand-free, and DHT-, MPA-, CYP-, RU486-, FLUT- or DHEA-bound). Thus, it shows the conformational specificity of the peptides. FIG. 4B is the converse of 4A. It compares the ability of six ligands (DHT, MPA, CYP, RU486, FLUT and DHEAs to interact with AR in the presence of each of four peptides (D30, 5G11, B8H3 and DHEA). Thus, it is a “fingerprint” of each of the six ligands using a four peptide panel. (Note that “1269” and D30″ refer to the same peptide.)

Once these peptides have been characterized for their ligand specificity (see Tables 502A/B), they can be used in a cell-based screening format to identify ligands for the androgen receptor. We have formatted such a 96-well assay using peptide B8E9 to identify ligands to the androgen receptor from a set of novel steroid compounds. The B8E9 peptide displays binding to the androgen receptor in the presence of both agonists and known antagonists, but not in the absence of ligand. Therefore, it is a useful tool to identify putative agonists and antagonists of AR. To increase the throughput of the existing 24-well assay to a 96-well format, a batch transfection of peptide-, receptor-, and reporter plasmids was performed. Trypsinized Huh-7 cells were transfected with all three plasmids in suspension using lipofection reagents and then seeded into the wells of 96-well plates. The compounds used in this screen were a collection of ˜160 novel steroids dispersed into individual wells of 2,96-well plates. The steroids were added to a final concentration of 1 uM and incubated with the cells overnight (˜18 hours) prior to performing luciferase assays to determine the reporter activity induced by each compound. FIG. 5 represents an example of this screen. Each point represents a unique compound arrayed in each well of a 96-well assay plate. Interaction of the peptide with the androgen receptor is measured by increased signal of the luciferase reporter activity and represents the presence of an androgen receptor ligand in the compound well. There is obvious sensitivity and specificity in this assay as evidenced by the variety of interaction between these steroids and the androgen receptor in the presence of the B8E9 peptide. Other AR conformation-specific peptides could be used in place of B8E9. This example clearly indicates that this peptide-based approach can identify compounds that interact with the androgen receptor in a high throughput and physiologically relevant manner.

We have moved the assay into 384-well plates for a high-throughput screen of compound libraries. To facilitate this, we prefer to follow the protocol below. We trypsinize the Huh-7 cells grown a large flask to make them non-adherent, and transfect the plasmids using lipofectamine 2000 reagent in a batch method prior to seeding the cells into either 96- or 384-well plates. The cells recover for at least 4 hours and compounds added and incubated overnight. The next morning luciferase assays are performed to determine the extent of peptide-protein interactions.

Preferred Procedure for 96-Well Plate:

1) Dilute 21 ul lipofectamine 2000 reagent into 400 ul OptiMEM-1 and incubate at RT for 5 minutes.

2) Dilute all the DNA in another 400 ul OptiMEM-1

3) Combine diluted reagent with diluted DNA, mix gently and incubate at RT for 20 minutes to allow DNA lipid complexes to form.

4) During the incubation time, trypsinize and count cells, spin down cells (20,000×96 per plate) and make a cell suspension so that the appropriate number of cells per well are contained in 100 ul of transfection medium.

5) Add the cell suspension to the DNA-LF2000 reagent complexes, mix gently and seed 100 ul to each well in solid white 96-well TC plates (Costar). Incubate at 37° C. for at least 4 hours (to overnight) in CO₂ incubator.

6) Aspirate DNA lipid reagent from wells and add 100 ul DMEM (phenol red free)+10% Charcoal-dextran treated FBS to each well. (optional: Let cells recover overnight).

7) Aspirate recovery medium and add phenol red-free medium without serum or antibiotics. Add compounds to cells and incubate at 37° C. for 4-24 hours.

8) Remove medium and wash cells 2× with PBS (no Mg++or Ca++) add 40 ul lysis buffer (Tropix Galacto-light Plus +DTT to 1 mM final as per directions) to each well and incubate RT 10 minutes with shaking.

9) Transfer 20 ul from each well to another solid white 96-well plate (non-treaten assay plate) for b-galactosidase assay. The remaining 20 ul is for luciferase assay.

10) Luciferase assay: (Tropix: Luc-Screen kit) Mix buffers 1 and 2 equal volume ahead of time and allow them to warm to RT. Add 20 ul of mixture directly to the 20 ul cell lysate, mix well and incubate RT for 10 minutes then read. (luminometer: 0.1 sec/well).

11) β-galactosidase assay: (Tropix Galacto-light Plus) follow manufacturer's protocol.

Examples of Possible Modifications:

Depending on our needs, we will often not normalize to β-galactosidase. We find good reproducibility in the assay when cells are transfected in a batch manner and then seeded into the wells. If no β-galactosidase assay is to be performed, step 8 may be changed to aspirating, washing and adding 20 ul of PBS and then going directly to step 10.

We have found that we can incubate the transfection reaction for 4 hours and then add compound directly to the transfection medium without aspirating or allowing cell recovery. In this case, the compounds are preferably then left on overnight prior to assaying luciferase activity.

Depending on the receptor being assayed, it may be preferably for the cells to be grown 24 hours prior to transfection in phenol red-free medium with charcoal dextran treated serum. We have found that ER is most influenced by this, whereas AR and GR are much less influenced by this treatment.

Cells can be seeded into 384-well plates by using a MultiDrop 384 instrument if performing a large number of assays.

### Example 503

### Glucocorticoid Receptor (GR)

We have performed a limited amount of in vivo screenings for peptides to GR in the yeast based system using full-length receptor and either dexamethasone or medroxyprogesterone acetate as ligands. We have a single peptide sequence that we have followed up on based on its specificity profile in yeast. This peptide was isolated on MPA liganded GR, and is termed GRMPA. Its sequence is as follows: EFVARYGQLLGWRHPCS (SEQ ID NO:268). When tested in yeast, the peptide interacted with GR in the presence of partial agonists (MPA, cortivazol, and deoxycorticosterone) and antagonists (RU486), not full agonists (fluticasone propionate and dexamethasone). However, when tested in mammalian cells the peptide interacted in the presence of all ligands (agonists, partial agonists, and antagonists).

We have isolated numerous peptides using phage display to the GR ligand binding domain and have formatted cell based assays in mammalian cells for screening large compound sets. We have formatted the assay as stated above for 96-well plates and for 384-well plates. To format the assay to 384-well plates the number of cells per well were scaled down to 5,000 Huh-7 cells per well. Similar ratios of DNA constructs and lipection reagents were used in both assays for transfections prior to seeding the cells into the assay plates. We have also carried out a screen of a 60,000 compound set with one of the phage display isolated peptides in this mammalian cell based format. This peptide interacts with the glucocorticoid receptor in the presence of both agonists (e.g. fluticasone propionate and dexamethasone) and antagonists (RU486). For the compound screen cells were transfected in suspension with DNA constructs as described above prior to seeding 5,000 cells per well. Compounds were added to 10 uM 6 hours after transfection and incubated overnight at 37° C. After compound incubation luciferase assays were carried out as per manufacturer's protocol.

Alternative Methods

In addition to the methods outlined above, one can also imagine additional formats as well as other types of cells that might be useful in intracellular peptide screening.

Method 1: Use of unfused nuclear receptor partner that interacts with nuclear receptor fusion and peptide fusion. To date, there are two broad categories of nuclear receptor, those belonging to the steroid family (ER, AR, GR, PR, MR) and those belonging to the heterodimer family (RAR, TR, VDR, LXR, FXR, etc.), but use RXR as the heterodimer partner. In general, members of the steroid receptor family undergo dimerization upon activation, while the other family appears to from functional dimers between an RXR receptor and one of the other members (TR, VDR, etc.). While members of this family can also homodimerize like the steroid receptor family, it appears that the heterodimer form may be the preferred (physiological) form of the receptor. The methods we have described for screening steroid receptors can equally be applied to members of the heterodimer family. The difference lies in the fact that one can identify peptides either to the unique receptor member (i.e. non-RXR) of the heterodimer pair (presumably as a homodimer in the absence of a partner receptor), or to the RXR or heterodimer partner of the receptor pair (but in the context of a heterodimer). This approach can also be extended to novel, as yet undiscovered, receptors. In this method, one can imagine being able to select peptides that interact with a partner of a known (or uncharacterized) nuclear receptor in the presence (or absence) of a ligand. In the example we envisage, a cloned receptor (e.g. TR) is ligated to the pGAD-C2 vector to create a fusion vector. In addition, another vector containing RXR cDNA, for example, is ligated to a eukaryotic promoter, for example, a constitutive actin promoter or an inducible alcohol dehydrogenase promoter. Both plasmids are transformed into yeast to create a stable cell line. A library of expressed peptides fused to the GAL4 DBD is then transformed into the stable line. Ligand, in this case thyroxine, might be added to the coexpressing cells, to select for peptides that interact in the presence of ligand. Ligand dependence is then determined for positive transformants as described above. True ligand dependent positives are extracted to obtain plasmids expressing desired peptides for retesting. These positives may represent selection of peptides either to the RXR or to the TR receptor in the situation described here. In addition to the Gal4-nuclear receptor fusion (e.g., encoded by the pGAD-C2-receptor, Gal4 activation domain fusion, the transformed yeast strain would have another plasmid expressing an unfused receptor partner (e.g. RXR, RAR, or other partner). Likewise, this method could be extended to nuclear receptors that do not generally share a heterodimer partner like RXR. This could include, for example, ER α and ERβ heterodimer pairs, or ERβ and AR heterodimer pairs, that might otherwise be difficult to form as full-length proteins in vitro. Since a receptor partner may assume different conformations depending on the ligand present (or not), peptides to the partner complex (i.e. fused receptor+unfused receptor) may be selected. This may be especially important for discovering new, biologically-relevant peptides that cannot otherwise be obtained by conventional protein production of the individual components of the partner pair.

One could further extend this approach to identifying peptides which bind proteins that interact with nuclear receptors, or any other set of proteins, in a ligand-dependent manner. In this case, one would transform yeast with vector constructs containing a nuclear receptor fused to an activation domain and an expression plasmid that encodes a known receptor binding protein (e.g. coactivators: SRC-1, GRIP-1; corepressors: NcoR; Associated proteins: ARA70, NF-κB, c-jun, TFIIB). The stably transformed yeast are then transformed with a library of peptide sequences that are a fusion with a Gal4 DNA binding domain. Colonies are then selected for ligand- and receptor binding protein-dependent growth. By this approach, conformational probes of biological function can be selected in vivo.

Method 2: Mating-type dependent selection of peptides. In addition to the general transformation protocol described in 502, it should also be possible to carry out a selection for interacting peptides where the nuclear receptor fusion vector is stably integrated into either the MAT a or MAT a mating type strain of yeast and a library of fusion peptide vectors are stably maintained in the other strain. Thus, the peptide library is expressed in one haploid strain mating type and the receptor in the other. When the strains mate, each of the resulting diploid cells coexpresses a library peptide and the receptor. In this way an entire library can be prepared and maintained as frozen stocks. The procedure is similar to that described in 501.2, except that pools of nuclear receptor and peptide-expressing cells would be dispensed into 96- or 384-well plates, and mating would proceed in the presence (and absence) of ligand. Growth in medium would be based on selection of markers requiring the union of both mating types. This method could be used both for selection of peptides to a receptor like AR, or used to identify peptides through a receptor partner, like RXR or RAR, as described in Method 1 above. The disadvantage of the method is the low mating frequency generally achieved. A method based on two-hybrid selection using mating type has been described by Buckholz, R., Simmons, C., Stuart, J. and Weiner, M., “Automation of Yeast Two-Hybrid Screening”, J. Molecular Microbiology and Biotechnology, vol. 1, p. 135-140.

Method 3: Use of other cell types to carry out peptide selections in vivo. While yeast cells are very useful genetic and biological tools for studying specific protein interactions, they lack many of the physiologically-relevant features that other eukaryotic cells contain, particularly as relate to nuclear receptors. Yeast have no nuclear receptor proteins or their associated coactivator proteins, so that peptide interactions must be extensively followed up in other cell types to determine their relevance. Alternatively, some cells, like Drosophila insect cells (S2), have properties more similar to mammalian cells (e.g. steroid receptors, coactivators, etc.) that may make them ideal for generating larger libraries of peptides, and so identifying relevant interactions. In addition, they have the interesting reported property of retaining up to several hundred copies of a transfected gene, rather than just one or two copies as for most mammalian cells.

We envisage a scenario where a large number of pM vector fusions with DNA encoding unbiased or biased random peptides are transfected into Drosophila S2 cells. Because of the relative ease of transfection, and the fact that each cell potentially could retain several hundred different copies of peptide sequences, it should be possible to create substantially larger peptide libraries in these cells than are possible with yeast (e.g. achieve a complexity of 10⁹-10¹⁰ vs. 10⁷). This would create a peptide library. A stable cell line in Drosophila would also be created containing a nuclear receptor, like AR, fused with the activation domain (e.g. pVP16). In addition, a reporter gene would be co-transfected with the receptor construct, preferably one in which the promoter contains a Gal4 (or other) DNA element driving a gene for a selectable marker, for example, a cell-surface protein. Such a stable line would then be used as the recipient of large scale transfections with peptide libraries in a vector that expresses a fusion with the Gal4 DNA binding domain, like pM vector.

In our projected scenario, it should be possible to create libraries of 10⁷-10⁸ (or more) cells each containing several hundred different peptide sequences, thus yielding substantially larger libraries than those available in yeast. After transfection of peptide vector into recipient cells, stable or transient cells expressing non-ligand dependent reporter protein would be removed (since they are auto-activators), and the remaining cells would be treated with ligand. After a treatment period, cells expressing reporter protein are again selected, propagated and their DNA would be extracted. As in conventional cell-based selection methods, the DNA from the cells would again be transfected into the AR/reporter stable cell line previously described. This would likely enrich for DNA demonstrating the desired properties and lead to enriched populations of cells showing desired receptor-peptide interactions. Ligand and non-ligand dependent reporter protein expression would again be used to select cells further enriched for the interacting peptide(s). The procedure is again repeated twice more and individual cells would be ultimately be sorted by FACS (fluorescence-activated cell sorting) or other sorting methodologies. Clones would be tested for ligand dependent production of reporter and the peptide sequence identified by PCR or cloning of the DNA. Peptide sequences could then be converted back to more conventional “Cellular Braille” assays we have described in 502, or the sequences can be converted to synthetic peptides for in vitro analysis.

Citation of documents herein is not intended as an admission that any of the documents cited herein is pertinent prior art, or an admission that the cited documents is considered material to the patentability of any of the claims of the present application. All statements as to the date or representation as to the contents of these documents is based on the information available to the applicant and does not constitute any admission as to the correctness of the dates or contents of these documents.

The appended claims are to be treated as a non-limiting recitation of preferred embodiments.

In addition to those set forth elsewhere, the following references are hereby incorporated by reference, in their most recent editions as of the time of filing of this application: Kay, Phage Display of Peptides and Proteins: A Laboratory Manual; the John Wiley and Sons Current Protocols series, including Ausubel, Current Protocols in Molecular Biology; Coligan, Current Protocols in Protein Science; Coligan, Current Protocols in Immunology; Current Protocols in Human Genetics; Current Protocols in Cytometry; Current Protocols in Pharmacology; Current Protocols in Neuroscience; Current Protocols in Cell Biology; Current Protocols in Toxicology; Current Protocols in Field Analytical Chemistry; Current Protocols in Nucleic Acid Chemistry; and Current Protocols in Human Genetics; and the following Cold Spring Harbor Laboratory publications: Sambrook, Molecular Cloning: A Laboratory Manual; Harlow, Antibodies: A Laboratory Manual; Manipulating the Mouse Embryo: A Laboratory Manual; Methods in Yeast Genetics: A Cold Spring Harbor Laboratory Course Manual; Drosophila Protocols; Imaging Neurons: A Laboratory Manual; Early Development of Xenopus laevis: A Laboratory Manual; Using Antibodies: A Laboratory Manual; At the Bench: A Laboratory Navigator; Cells: A Laboratory Manual; Methods in Yeast Genetics: A Laboratory Course Manual; Discovering Neurons: The Experimental Basis of Neuroscience; Genome Analysis: A Laboratory Manual Series; Laboratory DNA Science; Strategies for Protein Purification and Characterization: A Laboratory Course Manual; Genetic Analysis of Pathogenic Bacteria: A Laboratory Manual; PCR Primer: A Laboratory Manual; Methods in Plant Molecular Biology: A Laboratory Course Manual; Manipulating the Mouse Embryo: A Laboratory Manual; Molecular Probes of the Nervous System; Experiments with Fission Yeast: A Laboratory Course Manual; A Short Course in Bacterial Genetics: A Laboratory Manual and Handbook for Escherichia coli and Related Bacteria; DNA Science: A First Course in Recombinant DNA Technology; Methods in Yeast Genetics: A Laboratory Course Manual; Molecular Biology of Plants: A Laboratory Course Manual.

All references cited herein, including journal articles or abstracts, published, corresponding, prior or otherwise related U.S. or foreign patent applications, issued U.S. or foreign patents, or any other references, are entirely incorporated by reference herein, including all data, tables, figures, and text presented in the cited references. Additionally, the entire contents of the references cited within the references cited herein are also entirely incorporated by reference.

Reference to known method steps, conventional methods steps, known methods or conventional methods is not in any way an admission that any aspect, description or embodiment of the present invention is disclosed, taught or suggested in the relevant art.

The foregoing description of the specific embodiments will so fully reveal the general nature of the invention that others can, by applying knowledge within the skill of the art (including the contents of the references cited herein), readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one of ordinary skill in the art.

Any description of a class or range as being useful or preferred in the practice of the invention shall be deemed a description of any subclass (e.g., a disclosed class with one or more disclosed members omitted) or subrange contained therein, as well as a separate description of each individual member or value in said class or range.

The description of preferred embodiments individually shall be deemed a description of any possible combination of such preferred embodiments, except for combinations which are impossible (e.g, mutually exclusive choices for an element of the invention) or which are expressly excluded by this specification.

If an embodiment of this invention is disclosed in the prior art, the description of the invention shall be deemed to include the invention as herein disclosed with such embodiment excised.

The invention, as contemplated by applicant(s), includes but is not limited to the subject matter set forth in the appended claims, and presently unclaimed combinations thereof. It further includes such subject matter further limited, if not already such, to that which overcomes one or more of the disclosed deficiencies in the prior art. To the extent that any claims encroach on subject matter disclosed or suggested by the prior art, applicant(s) contemplate the invention(s) corresponding to such claims with the encroaching subject matter excised.

All references cited anywhere in this specification are hereby incorporated by reference, as are any references cited by said references.

TABLE A
List of Proteins for Fingerprinting Analysis:

| Receptors | Modulators of Activity¹ |
|---|---|
| Nuclear receptors | |
| Estrogen Receptor α and β | Estradiol (agon), tamoxifen |
| | (antag), ICI 182,780 |
| | (antag), Raloxifene, |
| | (antag), |
| Progesterone | Progestins, estrogens |
| | (agon), RU486 (antag), |
| | ZX98299, (antag), |
| | onapristone (antag) |
| Androgen | Dihydroxytestosterone |
| | (agon), hydroxyflutamide |
| | (antag) |
| Glucocorticoid | Cortisone (agon), |
| | dexamethasone (agon) |
| mineralocorticoid | Aldosterone (agon), |
| | spironolactone (antag) |
| Retinoic acid | 9-cis retinoic acid (agon) |
| Thyroid | Thyroid hormone (agon) |
| Vitamin D3 | Vitamin D3 (agon) |
| PPAR(s) | Eicosinoids (agon), oxidized |
| | LDL (agon) |
| LXR | Oxidized cholesterol |
| | metabolites (agon) |
| FXR | Farnesoid metabolites (agon) |
| BXR | 3-aminoethyl benzoate (agon) |
| SXR | Steroids (agon), |
| | phytoestrogens (agon), |
| | xenobiotics (agon) |
| Orphan Nuclear Receptors | |
| Nurr1 | |
| Nor1 | |
| NGF1-B | |
| ERR1 | |
| SHP | |
| HNF-4 | |
| Coup-TF II | |
| Tyrosine Kinase Receptors | |
| Epidermal | growth EGF (agon), ATP |
| factor | |
| Insulin | Insulin (agon), ATP |
| ¹ Antag = antagonist of receptor | |
| agon = agonist of receptor | |
| Platelet | derived PDGF (agon), ATP |
| growth factor | |
| G-Protein Coupled Receptors | |
| β-adrenergic receptor | Isopreterenol (agon), |
| | alprenolol (antag) |
| Rhodopsin | |
| Dopamine D2 | Dopamine (agon), haloperidol |
| | (antag) |
| opiod | Leu-enkephalin (agon), |
| | Naltrindole (antag) |
| Endothelin | Endothelin 1 (agon), BQ-123 |
| | (antag) |
| Erythropoietin receptor | Erythropoietin |
| FAS ligand receptor | FAS ligand |
| Interleukin receptor | Interferon (agon) IL-6 |
| | (agon) |
| Signal Transduction Proteins | |
| Kinases | |
| Protein Kinases | |
| Protein kinase C | diacylglycerol (agon), |
| | staurosporine (antag) |
| Tyrosine kinase | ATP, genistein (antag) |
| Serine kinase | ATP |
| Threonine kinase | ATP |
| Nucleotide kinase | ATP |
| Polynucleotide kinase | ATP, DNA, PO₄ |
| Phosphatase | |
| Protein Phosphatase | |
| Serine/threonine | |
| Tyrosine | |
| Nucleotide phosphatase | |
| Acid phosphatase | |
| Alkaline phosphatase | |
| pyrophosphatase | |
| Cell Cycle Regulators | |
| Cyclin CDK-2 | |
| CDC2 | |
| CDC25 | |
| p53 | |
| Retinoblastoma | |
| GTPases | |
| Large G proteins | |
| Gαs | suramin (antag), |
| | mastoparin (agon) |
| Small G Proteins | GAPs (ag), GEF (antag) |
| Rac | |
| Rho | |
| Rab | |
| Ras | |
| Proteases | |
| Endoprotease | |
| Exoprotease | |
| Metalloprotease | |
| Serine protease | |
| Cysteine protease | |
| Nucleases | |
| Polymerases | |
| Ion Channels | |
| Chaperonins | |
| Heat shock Proteins | |
| Viral Proteins | |
| Deaminases | |
| Nucleases | |
| Deoxyribonuclease | |
| Ribonuclease | |
| Endonucleases | |
| Exonucleases | |
| Polymerases | |
| DNA dependent RNA polymerase | |
| DNA dependent DNA polymerase | |
| Telomerase | |
| Primase | |
| Helicase | |
| Dehydrogenase | |
| Aminoacyl tRNA synthetases | |
| Transferases | |
| Peptidyl transferase | |
| Transaminase | |
| Glycosyltransferase | |
| Ribosyltransferase | |
| Acetyl transferases | |
| Acyltransferases | |
| Hydrolases | |
| Carboxylases | |
| Isomerases | |
| Dismutase | |
| Rotase | |
| Topoisomerase | |
| Glycosidase | |
| Endoglycosidase | |
| Exoglycosidase | |
| Deaminase | |
| Lipases | |
| Esterases | |
| Sulfatases | |
| Cellulase | |
| Lyases | |
| Reductases | |
| Synthetase | |
| DNA binding proteins | |
| RNA binding proteins | |
| Nuclear receptor coactivators | |
| Ligases | |
| RNA | |
| DNA | |
| Tumor suppressor | |
| Adhesion molecule | |
| Oxygenase | |
| Peroxidase | |
| Transporters | |
| Electron transporters | |
| Protein transporters | |
| Peptide transport | |
| Hormone transport | |
| Serotonin | |
| DOPA | |
| Nucleic acid transport | |
| Transcription factors | |
| Neurotransmitters | |
| Information carrier/storage | |
| Antigen recognition protein | |
| MHC I complex | |
| MHC II complex | |

TABLE B
Target Tissues

Circulatory and Lymphatic Systems
Heart
Walls
Valves
Blood Vessels
Blood Cells
Erythrocytes
Platelets
Leukocytes
Lymph Nodes
Lymphatic Vessels
Spleen
Thymus
Tonsils
Respiratory System
Lungs
Trachea
Bronchi
Bronchioles
Alveoli
Pleura
Pharynx
Larynx
Trachea
Endocrine System
Pituitary Gland
Thyroid Gland
Parathyroid Gland
Adrenal Gland
Adrenal Medulla
Adrenal Cortex
Pancreas
Islets of Langerhans
Liver
Gall Bladder
Mammary Glands
Central Nervous System
Brain
Neurons
Glial Cells
Spinal Cord
Nerves
Peripheral Nervous System
Eye
Retina
Lens
Ear
Eardrum
Ampullae
Spiral organ of Corti
Nose
Olfactory bulbs
Tongue
taste buds
Digestive System
Tongue
Salivary Gland
Pharynx
Esophagus
Stomach
Small Intestine
Large Intestine
Urinary System
Kidney
nephrons
Bladder
Male Reproductive System
testes
prostate gland
bulbourethral (Cowper's) glands
penis
sperm cells
Musculoskeletal System
bones (various)
bone marrow
joints (various)
muscles (various)
ligaments (various)
Female Reproductive System
Ovaries
Uterus
Bartholin's Glands
Paraurethral Glands
Egg Cells
Integumentary System
Skin
epidermis
dermis
hypodermis
sweat glands
sebaceous glands
hair
nails

TABLE 1

Peptides that Bind to the Unligandeci (unactivat-
ed) Estrogen Receptor

| | | |
|---|---|---|
| Sequence | SEQ ID NO: | Phage # |

| | | |
|---|---|---|
| S R W E S P L G T W E W S R | 1 | 4 |
| S A A P R T I S H Y L M G G | 2 | 48 |
| S S W V R L S D F P W G V S R | 3 | 1 |
| S S W D R L S D F P W G V S R | 4 | 2 |
| S S W I R L R D L P W G E S R | 5 | 3 |
| S S W V L L R D L P W G S R | 6 | 31 |
| S S W V V L R D L P W G S R | 7 | 29 |
| S S C K W Y E K C S G L W S R | 8 | 7 |
| S S G I C F F W D G C F E S R | 9 | 35 |
| S R N L C F F W D D E Y C S R | 10 | 41 |
| H H H R H P A H P H T Y G G | 11 | 47 |

TABLE 2

Peptides that Bind to the Estradiol Activated Re-
ceptor

| | | |
|---|---|---|
| Sequence | SEQ ID NO: | Phage # |

| | | |
|---|---|---|
| S R A G L L S D L L E G K S R | 12 | 1/2 |
| S S R S L L R D L L M V D S R | 13 | 6 |
| S S N K L L Y N L L K M E S R | 14 | 22 |
| S S K S L L L N L L S T P S R | 15 | 23 |
| H S F P R E S L L V R L L Q G G | 16 | 42 |
| S R L E M L L R S E T D F S R | 17 | 3 |
| S R L E E L L K W G S V T S R | 18 | 11 |
| S R L E Q L L K E E F S Y S R | 19 | 21 |
| S R L E Q L L R S E P D F S R | 20 | 27 |
| S R L E D L L R A P F T T S R | 21 | 28 |
| S R L E S L L R F G Q L D S R | 22 | 29 |
| S S R L L S L L V G D F N S R | 23 | 19/20 |
| S R L E E L L L G T N R D S R | 24 | 30 |
| S R L K E L L L L P T D L S R | 25 | 15 |
| S R L E C L L E G R L N C S R | 26 | 34 |
| S S K L Y C L L D E S Y C S R | 27 | 35 |
| S R L S C L L M G F E D C S R | 28 | 36 |
| S S K L I R L L T S D E E L S R | 29 | 37 |
| S S R L M E L L Q E G Q G W S R | 30 | 40 |
| S S N H Q S S R L I E L L S R | 31 | 4 |
| S S R L W Q L L A S T D T S R | 32 | 16 |
| S S N S M L W K L L A A P S R | 33 | 13/14 |
| S S K T L W R L L E G E R S R | 34 | 17 |
| S R A G P V L W G L L S E S R | 35 | 32 |
| S S L T S R D F G S W Y A S R | 36 | 5 |
| S S W V R L S D F P W G V S R | 37 | 24/25 |
| S S E Y C F Y D S A H C S R | 38 | 33 |
| S R S L L E C H L M G N C S R | 39 | 7 |
| S S E L L R W H L T R D T S R | 40 | 8 |
| S R L E Y W L K W E P G P S R | 41 | 12 |
| S R S D S I L W R M L S E S R | 42 | 31 |
| S S K G V L W R M L A E P V S R | 43 | 38/39 |
| H S H G P L T L N L L R S S G G | 44 | 41 |
| S S A G G G A P A G S T P S R | 45 | 26 |

Other ER binding peptides include

| | |
|---|---|
| SSKYSYSRSSEGHSR | (SEQ ID NO:46) |
| SSYQWETHSDKWRSR | (SEQ ID NO:47) |
| SSVTKKALTIAKDSR | (SEQ ID NO:48) |

The latter two are weak binders of ER in presence of estradiol.

TABLE 3

Phage/Peptide Classification

| | | |
|---|---|---|
| | SEQ | |
| | ID | |
| method | NO: | #and isolation |

| | | | |
|---|---|---|---|
| Class 1 | | | |
| S S N H Q S S R L I E L L S R | 49 | #4 E R | + |
| | | estradiol | |
| S R L K E L L L L P T D L S R | 50 | #15 E R | + |
| | | estradiol | |
| S S K L Y C L L D E S Y C S R | 51 | #35 E R | + |
| | | estradiol | |
| H G P L T L N L L R S S G G | 52 | #41 E R | + |
| | | estradiol | |
| S R L E Y W L K W E P G P S R | 53 | #12 E R | + |
| | | estradiol | |
| Class 2 | | | |
| S S C K W Y E K C S G L W S R | 54 | #7 ER | |
| S S E Y C F Y W D S A H C S R | 55 | #33 E R | + |
| | | estradiol | |
| S S W V L L R D L P W G S R | 56 | #31 ER | |
| S S W V R L S D F P W G V S R | 57 | #24 E R | + |
| | | estradiol | |
| Class 3 | | | |
| S S L T S R D F G S W Y A S R | 58 | # E R | + |
| | | estradiol | |
| Class 4 | | | |
| S R T W E S P L G T W E W S R | 59 | #13 ER | |
| Class 5 | | | |
| S A A C A T I S H Y L M G G | 60 | #48 ER | |

TABLE 4
Characteristics of the 5 Phage Classes

| | | | | Competition |
|---|---|---|---|---|
| | Affinity for | Affinity for | Effect of | with |
| | unliganded | unliganded | Agonist | LXXLL |
| | ER α | ER 13 | (Estradiol) | peptide |
| Class | + | +++ | ↑ binding | + α |
| 1 | | | to α & β | + β |
| Class | +++ | ++ | No effect | − α |
| 2 | | | | − β |
| Class | ++ | + | ↑ binding | − α |
| 3 | | | to α no | − β |
| | | | effect on | |
| | | | β | |
| Class | +++ | ++ | ↓ binding | + α |
| 4 | | | to α no | − β |
| | | | effect on | |
| | | | β | |
| Class | ++(+) | +++ | ↓ binding | + α |
| 5 | | | to α & β | − β |

TABLE 7

New Erα Peptide Sequences Immobilized on Plastic

| | | | | |
|---|---|---|---|---|
| | | | Isolated in the | |
| Peptide | | SEQ ID | presence of | SERM present when peptide was |
| name | Peptide Sequence | NO: | receptor form | identified |

| | | | | |
|---|---|---|---|---|
| 1PT | SRNLCFFWDDEYCSR | 74 | α | Tamoxifen & ICI 182,780 |
| 2PT | SWDMHQFFWEGVSR | 75 | α | Tamoxifen |
| 3PT | SRWHGTLEWQDEQSR | 76 | α | Tamoxifen |
| 4PT | SSCKWYEKCSGLWSR | 77 | α | Tamoxifen & ICI 182,780 |
| 5PT | SSRMGHVWYDWTFSR | 78 | α | Tamoxifen |
| 6PT | SSRLLGDFGGSVVSR | 79 | α | Tamoxifen |
| 7PT | SSKYVFGFQVAGGSR | 80 | α | Tamoxifen |
| 8PT | SSWAGIKFGKPPHSR | 81 | α | Tamoxifen |
| 9PT | SSSWSYGKPTFLSSR | 82 | α | Tamoxifen |
| 10PT | SRDTGDMWWGRGGSR | 83 | α | Tamoxifen |
| 11PT | SSGRYDPFVLNAASR | 84 | α | Tamoxifen |
| 12PT | SSSPWWSFNLRDMSR | 85 | α | Tamoxifen |
| 13PT | SSWPYLPKREEWASR | 86 | α | Tamoxifen |
| 14PT | SSGWIEQKLRGSFSR | 87 | α | Tamoxifen |
| 15PT | SSSATSIKVQYQISR | 88 | α | Tamoxifen |
| 16PT | SSYLTLGKSMMAISR | 89 | α | Tamoxifen |
| 17PT | SSWHSRWDLALGFSR | 90 | α | Tamoxifen |
| 18PT | SSGYWGGWDYGAGSR | 91 | α | Tamoxifen |
| 19PT | SRDNCGAGLWAGCSR | 92 | α | Tamoxifen |
| 1PI | SSSTPGWWEWDWASR | 93 | α | ICI 182,780 |
| 2PI | SSYWDGSWRRKETCVSCSR | 94 | α | ICI 182,780 |
| 3PI | SSRTAEDYCFFADDYWCSR | 95 | α | ICI 182,780 |
| 4PI | SSRALALFPVGMESR | 96 | α | ICI 182,780 |
| 5PI | SSDCESLTSYPHLKALCSR | 97 | α | ICI 182,780 |
| 6PI | SSTATALRDRLAYSR | 98 | α | ICI 182,780 |
| 7PI | SSGKTREHYREGTSR | 99 | α | ICI 182,780 |

TABLE 8

New ERα-ERE Peptide Sequence Information

| | | | | |
|---|---|---|---|---|
| | | | Isolated in the | |
| Peptide | | SEQ ID | presence of | SERM present when |
| name | Peptide Sequence | NO: | receptor form | peptide was identified |

| | | | | |
|---|---|---|---|---|
| E1-1 | HSHNHHSPWLFRLLGG | 100 | α | Estradiol |
| E1-3 | HSHPHHSHLLYKLMGG | 101 | α | Estradiol |
| E1-4 | HSHPLPPLLSRLLTGG | 102 | α | Estradiol |
| E1-7 | SRLTCLLQSNGWDSEQCSR | 103 | α | Estradiol |
| I4-10 | SSLTSRDFGSWYASR | 104 | α | ICI |
| T3-1 | SRTLQLDWGTLYSR | 105 | α | Tamoxifen |
| T1-10 | SRLPPSVFSMCGSEVCLSR | 106 | α | Tamoxifen |
| T2-10 | SRFEIWKPEPGCVSSLENWEPGKRV | | α | Tamoxifen |
| | CSR | 107 | | |
| T3-11 | SRVFGVSGGEVVLINGSSR | 108 | α | Tamoxifen |
| 1R | SRLCFGDWCMLGGVDVLSR | 109 | α | Raloxifen |
| 2R | SSLNMVVDTPWCGKWVCSR | 110 | α | Raloxifen |
| 3B | SSRPDAAFFGAKLSR | 111 | α | Buffer |
| 4B | SSRPSPSFWEKQLSR | 112 | α | Buffer |
| 5B | SSRPTAEWFRENLSR | 113 | α | Buffer |
| 6B | SRWWDTSWWLEELSR | 114 | α | Buffer |
| 1B | SSRIADLFWRLEPSR | 115 | α | Buffer |
| 7B | SRSYHGEWGVWTLSR | 116 | α | Buffer |
| 10B | SSDWCFGWGGWCASEAVSR | 117 | α | Buffer |
| 9B | SRNWDWAALELLPYPHPSR | 118 | α | Buffer |
| 1E | SSLTSRDFGSWYASR | 119 | α | Estradiol |
| 2E | SRSPILTHLLSLGSR | 120 | α | Estradiol |
| 3E | SSTGILWKLLTAESR | 121 | α | Estradiol |
| 9E | SSHGILWRLLSEGSR | 122 | α | Estradiol |
| 11E | SRSDSILWRMLSESR | 123 | α | Estradiol |
| 4E | SRLVALLKSPWSVSR | 124 | α | Estradiol |
| 5E | SRLEELLLMDFWRSR | 125 | α | Estradiol |
| 6E | SSKLWQLLSSPIDSR | 126 | α | Estradiol |
| 14E | SSKLYCLLDESYCSR | 127 | α | Estradiol |
| 7E | SRSLLMDMLMSDDYVTVSR | 128 | α | Estradiol |
| 8E | SSRLLACELMYEDADVCSR | 129 | α | Estradiol |
| 15E | HSHSPLLMALLAPPGG | 130 | α | Estradiol |
| 10E | SRLEYYLRLGTYESR | 131 | α | Estradiol |
| 13E | SSCLREILLYGACSR | 132 | α | Estradiol |
| 16E | SSRTAEDYCFFADDYWCSR | 133 | α | Estradiol |
| 17E | SSLRCYLSSSKVDQWACSR | 134 | α | Estradiol |
| 18E | SSYKPHSLLEWHLLGGTSR | 135 | α | Estradiol |

TABLE 9

New ERβ-ERE Peptide Sequence Information

| | | | | |
|---|---|---|---|---|
| | | SEQ ID | Isolated in the presence | SERM present when |
| Peptide name | Peptide Sequence | NO: | of receptor form | peptide was identified |

| | | | | |
|---|---|---|---|---|
| 1B-β | SRLHCLLDSSYCSSR | 136 | β | Buffer |
| 2B-β | SRLHCLLDSSYCSSR | 137 | β | Buffer |
| 3B-β | SSWPNPTFWERQLSR | 138 | β | Buffer |
| 4B-β | SYSKEWFEERLNSR | 139 | β | Buffer |
| 5B-β | SSSMMREFFERELSR | 140 | β | Buffer |
| 6B-β | SSGLPPNFERMLKSR | 141 | β | Buffer |
| 7B-β | SSGPWLMHYLGGGSR | 142 | β | Buffer |
| 8B-β | SSTSWLHHYLMGTSR | 143 | β | Buffer |
| 9B-β | SRGGGECLGPWCLSR | 144 | β | Buffer |
| 12B-β | SSEACVGRWMLCEQLGVSR | 145 | β | Buffer |
| 14B-β | SSQVWPGPWRLVESR | 146 | β | Buffer |
| 16B-β | SSSLGPWRLSELESR | 147 | β | Buffer |
| 17B-β | SSSGPWRWGLSIESR | 148 | β | Buffer |
| 18B-β | SRECVGGWCLAELSR | 149 | β | Buffer |
| 19B-β | SSIPPRSWWLSQLSR | 150 | β | Buffer |
| 20B-β | SSWPGAEWFKEQLSR | 151 | β | Buffer |
| 21B-β | SSKLYCLLDESYCSR | 152 | β | Buffer |
| 23B-β | HSYSSHPLLLSYLWGG | 153 | β | Buffer |
| 24B-β | HSWLGPWRLSSIDLGG | 154 | β | Buffer |
| 25B-β | HSTDMGWLRPWRLLGG | 155 | β | Buffer |
| 1T-β | SSVFTIMDGKVALSR | 156 | β | Tamoxifen |
| 2T-β | SRPYCLGDVWCLDSR | 157 | β | Tamoxifen |
| 4T-β | SREWEDGFGGRWLSR | 158 | β | Tamoxifen |
| 5T-β | SSWNSREFFLSQLSR | 159 | β | Tamoxifen |
| 6T-β | SSTTMFDFFYERLSR | 160 | β | Tamoxifen |
| 7T-β | SSARPWWLQFEGSSR | 161 | β | Tamoxifen |
| 8T-β | SSQEEWLLPWRLASR | 162 | β | Tamoxifen |
| 9T-β | SRLPPSVFSMCGSEVCLSR | 163 | β | Tamoxifen |
| 10T-β | SSGPFYVGGMLWPADCLSR | 164 | β | Tamoxifen |
| 12T-β | SREGWMGPWRLADSR | 165 | β | Tamoxifen |
| 13T-β | SRNECIGPWCLTISR | 166 | β | Tamoxifen |
| 14T-β | SSPGSREWFKDMLSR | 167 | β | Tamoxifen |
| 15T-β | SSVASREWWVRELSR | 168 | β | Tamoxifen |
| 16T-β | SRMFQVCGDEVCLRSR | 169 | β | Tamoxifen |
| 17T-β | SSDLHRDCLGVWCLSR | 170 | β | Tamoxifen |
| 18T-β | SRLNGVFCHDSSDLWVCSR | 171 | β | Tamoxifen |
| 20T-β | SRPGCLRGVWCLADTPPSR | 172 | β | Tamoxifen |
| 21T-β | SSRLVPHSFWLDGLMHGSR | 173 | β | Tamoxifen |
| 22T-β | SSISTYHMGEWFYAMLSSR | 174 | β | Tamoxifen |
| 23T-β | SSDLYSQMREFFQINLSR | 175. | β | Tamoxifen |
| 1E-β | SSRGLLWDLLTKDSR | 176 | β | Estradiol |
| 2E-β | SRHGILWDLLQGDSR | 177 | β | Estradiol |
| 3E-β | SRLHDLLLRDESPSR | 178 | β | Estradiol |
| 4E-β | SRDWRSGFLYELLSR | 179 | β | Estradiol |
| 5E-β | SSDTRSRLYELLSSSYTSR | 180 | β | Estradiol |
| 6E-β | SRLEELLRVGVLTSR | 181 | β | Estradiol |
| 7E-β | SRLEDLLRGDSKPQSR | 182 | β | Estradiol |
| 8E-β | SSPTGHRLLESLLLNSNSR | 183 | β | Estradiol |
| 9E-β | SSILERLLGGGSAETV | 184 | β | Estradiol |
| 10E-β | SRSPILWHLLQDGSR | 185 | β | Estradiol |
| 11E-β | SSRTPILFSLLETSR | 186 | β | Estradiol |
| 12E-β | SSIKDFPNLISLLSR | 187 | β | Estradiol |
| 13E-β | SSGSSAGRLMMLLQDGVSR | 188 | β | Estradiol |
| 14E-β | SREGLLMRLLIGDSR | 189 | β | Estradiol |
| 15E-β | SSHCHTRLCSLLTSR | 190 | β | Estradiol |
| 16E-β | SSRLLCLLDAGQCSR | 191 | β | Estradiol |
| 17E-β | SRNLLCLLDQEACSR | 192 | β | Estradiol |
| 18E-β | SSLKCLLNSNFCSR | 193 | β | Estradiol |
| 19E-β | SSLKCLLQSSPQKQPFCSR | 194 | β | Estradiol |
| 20E-β | SSRTLLEHYLLGGSR | 195 | β | Estradiol |
| 21E-β | SSAGLLEDMLRSRSR | 196 | β | Estradiol |
| 22E-β | SSRCSSLLCEMLIQTKESR | 197 | β | Estradiol |
| 23E-β | SSLQAGSWLMHYLRGGDSR | 198 | β | Estradiol |
| 24E-β | SRPEGSSWLLHYLSR | 199 | β | Estradiol |
| 25E-β | SSRTLLEHYLLGGSR | 200 | β | Estradiol |
| 26E-β | SRWWLDDHELLLYSSR | 201 | β | Estradiol |
| 27E-β | SSRTLYCHLTSSNPEWCSR | 202 | β | Estradiol |
| 28E-β | SSTRLMCWLGSADTSHCSR | 203 | β | Estradiol |
| 29E-β | SSYDWQCPSWYCPAPPSSR | 204 | β | Estradiol |
| 30E-β | SSTTWRCPEWYCGSR | 205 | β | Estradiol |
| 31E-β | SSWDFRVPWWYNNSR | 206 | β | Estradiol |
| 32E-β | SSQWQAPWWYIDASR | 207 | β | Estradiol |
| 33E-β | SSRPSFTIPWWFDDPSRSR | 208 | β | Estradiol |
| 34E-β | SSYEIPKWALQWLSR | 209 | β | Estradiol |
| 35E-β | SSLDLSQFPMTASFLRESR | 210 | β | Estradiol |

TABLE 10
Panel Peptides (see Tables 14A, 14B)

| | | | | |
|---|---|---|---|---|
| α/β I, | SSNHQSSRLIELLSR | (AB1) | [17βestradiol] | (SEQ ID NO:211) |
| α/β II, | SAPRATISHYLMGG | (AB2) | [no modulator] | (SEQ ID NO:212) |
| α/β III, | SSWDMHQFFWEGVSR | (AB3) | [4-OH tamoxifen] | (SEQ ID NO:213) |
| α/β IV, | SRLPPSVFSMCGSEVCLSR | (AB4) | [same] | (SEQ ID NO:214) |
| α/β V, | SSPGSREWFKDMLSR | (AB5) | [same] | (SEQ ID NO:215) |
| α I, | SSEYCFYWDSAHCSR | (A1) | [17β-estradiol] | (SEQ ID NO:216) |
| α II, | SSLTSRDFGSWYASR | (A2) | [17β-estradiol] | (SEQ ID NO:217) |
| α III, | SRTWESPLGTWEWSR | (A3) | [no modulator] | (SEQ ID NO:218) |
| β I, | SREWEDGFGGRWLSR | (B1) | [4-OH tamoxifen] | (SEQ ID NO:219) |
| β II, | SSLDLSQFPMTASFLRESR | (B2) | [17β-estradiol] | (SEQ ID NO:220) |
| β III, | SSEACVGRWMLCEQLGVSR. | (B3) | [no modulator] | (SEQ ID NO:221) |

Alternative name parenthesized. Modulator used to isolate peptide in brackets.

| | | Modulator (SERM) present during binding | | | | | | | | | | | |
|---|---|---|---|---|---|---|---|---|---|---|---|---|---|
| | | | | | | 4-OH | | | | | 16a- | | |
| | | | | | Tamoxi- | | | | | ICI | OH | | Proges- |
| Class | PeptideNa | buffer | Estradiol | Estriol | Premarin | fen | Nafoxidine | Clomiphene | Raloxifene | 182,780 | Estrone | DES | terone |

Table 14A: Class Specific Fingerprint on ERα

| | | | | | | | | | | | | | |
|---|---|---|---|---|---|---|---|---|---|---|---|---|---|
| α/βI | #4 ER./E2 | 1+ | 6+ | 4+ | 2+ | 1+ | 1+ | 1+ | 1+ | 1+ | 2+ | 2+ | 1+ |
| α/β_II | #48 ER | 7+ | 2+ | 4+ | 2+ | 1+ | 1+ | 1+ | 1+ | 1+ | 2+ | 2+ | 6+ |
| α/β_II | 2PT | 1+ | 1+ | 1+ | 2+ | 7+ | 4+ | 6+ | 4+ | 1+ | 2+ | 1+ | 2+ |
| α/β_I | 9Tβ | 1+ | 1+ | 1+ | 1+ | 6+ | 4+ | 4+ | 2+ | 0 | 1+ | 1+ | 1+ |
| α/β_V | 14Tβ | 1+ | 1+ | 1+ | 1+ | 1+ | 1+ | 2+ | 1+ | 1+ | 1+ | 1+ | 1+ |
| α_I | #33 R/E2 | 7+ | 7+ | 7+ | 6+ | 7+ | 7+ | 7+ | 7+ | 7+ | 6+ | 6+ | 6+ |
| α_II | #5 ER/E2 | 1+ | 6+ | 5+ | 6+ | 5+ | 4+ | 5+ | 4+ | 6+ | 5+ | 4+ | 1+ |
| α_III | #13 ER | 5+ | 2+ | 2+ | 2+ | 6+ | 2+ | 5+ | 2+ | 2+ | 2+ | 3+ | 4+ |

Table 14B: Class Specific Fingerprint on ERβ

| | | | | | | | | | | | | | |
|---|---|---|---|---|---|---|---|---|---|---|---|---|---|
| α/βI | #4 ER./E2 | 2+ | 7+ | 7+ | 6+ | 0 | 1+ | 0 | 0 | 0 | 5+ | 5+ | 1+ |
| α/β_II | #48 ER | 7+ | 2+ | 6+ | 4+ | 1+ | 4+ | 1+ | 4+ | 2+ | 3+ | 3+ | 6+ |
| α/β_II | 2PT | 2+ | 1+ | 1+ | 1+ | 7+ | 3+ | 5+ | 6+ | 1+ | 1+ | 1+ | 1+ |
| α/β_I | 9Tβ | 2+ | 1+ | 1+ | 1+ | 7+ | 5 | 5+ | 4+ | 1+ | 1+ | 1+ | 1+ |
| α/β_V | 14Tβ | 1+ | 1+ | 1+ | 1+ | 7+ | 3+ | 5+ | 2+ | 0 | 1+ | 1+ | 1+ |
| βI | 4Tβ | 6+ | 3+ | 2+ | 7+ | 7+ | 4+ | 3+ | 4+ | 0 | 2+ | 4+ | 5+ |
| βI | 35Eβ | 1+ | 5+ | 6+ | 4+ | 0 | 0 | 0 | 0 | 0 | 3+ | 3+ | 0 |
| βIII | 12Bβ | 7+ | 7+ | 7+ | 7+ | 1+ | 5 | 3+ | 3+ | 1+ | 7+ | 7+ | 5+ |

Notes to Table 14:
Fingerprint analysis of estrogen receptor modulators on (A) ER α and (B) ER β. Immobilized ER was incubated with estradiol (1 μM), estriol (1 μM), premarin (10 μM), 4-OH tamoxifen (1 μM), nafoxidine (10 μM), clomiphene (10 μM), raloxifene (1 μM), ICI 182,780 (1 μM), 16α-OH estrone (10 μM), DES (1 μM) or progesterone (1 μM). Phage ELISAs were conducted as described.

TABLE 15a
Binding of the peptide probes to ERα in the presence of modulators

| | α/βI | | α/βIII | | α/βIV | | α/βV | | αII | |
|---|---|---|---|---|---|---|---|---|---|---|
| Peptide Probe | Equiv.^{a} | EC50^{b} | Equiv. | EC50 | Equiv. | EC50 | Equiv. | EC50 | Equiv. | EC50 |
| Buffer | 0 | | 0 | | 0 | | 0 | | 0 | |
| 17β-Estradiol | 100 | 8.0 | −66 | 18.0 | −43 | 8.1 | 0 | | 100 | 17.5 |
| 17α-Estradiol | 53 | 10.0 | −61 | 88.0 | −54 | 5.9 | 0 | | 80 | 9.6 |
| Estriol | 65 | 8.1 | −59 | 19.2 | −28 | 44.9 | 0 | | 62 | 11.8 |
| 4-OH Tamoxifen | 0 | | 100 | 54.9 | 100 | 59.6 | 100 | 30.9 | 38 | 41.7 |
| Nafoxidine | 0 | | 23 | 292.1 | 13 | 372.2 | 0 | | 32 | 39.0 |
| Clomiphene | 0 | | 37 | 143.2 | 19 | 708.5 | 19 | 282.1 | 56 | 118.9 |
| Raloxifene | 0 | | 51 | 49.2 | 0 | | 0 | | 44 | 41.7 |
| ICI 182,780 | 0 | | −100 | 25.8 | −100 | 24.7 | 0 | | 56 | 28.5 |
| Diethylstilbesterol | 71 | 13.4 | −53 | 29.7 | 0 | | 0 | | 69 | 15.8 |
| GW7604 | 0 | | 0 | | 0 | | 0 | | 35 | 8.4 |
| ^{a}Equivalency may be positive or negative. These are both expressed in relative (percentage terms) but the positive and negative standards (100 | | | | | | | | | | |
| # and −100% marks) are set differently. Thus, the positive and negative values are scaled differently. Positive equivalency is defined as the | | | | | | | | | | |
| # maximum stimulation achieved with a given compound as a percentage of the maximum stimulation achieved with the positive modulator used | | | | | | | | | | |
| # for isolation of a given peptide probe (see Table 10). Negative values indicates that an increase in the concentration of a compound results in a | | | | | | | | | | |
| # reduction of the binding of the peptide probe as compared to the binding of the probe in buffer. These are expressed as a percentage of the | | | | | | | | | | |
| # reduction by ICI 182,780. For αII, ICI 182,780 acts as an agonist, and its equivalency is therefore stated as a percentage of the reference modulator | | | | | | | | | | |
| # β estradiol. Results for αIII were zero in all cases. | | | | | | | | | | |

^{b}EC50 is defined as the concentration in nanomolar of a given compound required to achieve fifty percent of the maximal signal for that compound.

TABLE 15b
Binding of the peptide probes to ERβ in the presence of modulators

| | α/βI | | α/βIII | | α/βIV | | α/βV | | βI | | βIII | |
|---|---|---|---|---|---|---|---|---|---|---|---|---|
| Peptide Probe | Equiv.^{a} | EC50^{b} | Equiv. | EC50 | Equiv. | EC50 | Equiv. | EC50 | Equiv. | EC50 | Equiv. | EC50 |
| Buffer | 0 | | 0 | | 0 | | 0 | | 0 | | 0 | |
| 17β-Estradiol | 100 | 21.8 | −71^{c} | 5.7 | −84 | 26.7 | 0 | | −69 | 12.8 | 100 | 17.0 |
| 17α-Estradiol | 44 | 8.8 | −78 | 7.1 | −82 | 12.9 | 0 | | −74 | 10.1 | 42 | 6.7 |
| Estriol | 81 | 19.5 | −57 | 15.8 | −75 | 12.4 | 0 | | −96 | 20.7 | 77 | 11.7 |
| 4-OH Tamoxifen | | | 100 | 37.3 | 100 | 179.8 | 100 | 50.0 0 | 100 | 20.6 | −100 | 34.4 |
| Nafoxidine | | | 27 | 231.7 | 0 | | 0 | | −44 | 320.5 | 0 | |
| Clomiphene | | | 34 | 82.2 | 0 | | 13 | 149.8 | −62 | 135.1 | −61 | 122.5 |
| Raloxifene | | | 77 | 90.1 | 0 | | 0 | | −53 | 89.9 | −71 | 156.2 |
| ICI 182,780 | | | −100 | 18.1 | −100 | 35.3 | 0 | | −100 | 28.9 | −100 | 48.4 |
| Diethylstilbesterol | 68 | 33.9 | −78 | 14.5 | −96 | 17.8 | 0 | | −59 | 11.1 | 86 | 25.4 |
| GW 7604 | 0 | | −86 | 4.2 | 74 | 3050.1 | 0 | | 159 | 3.3 | −106 | 7.7 |
| ^{a}Positive equivalency is defined as the maximum stimulation achieved with a given compound as a percentage of the maximum stimulation | | | | | | | | | | | | |
| # achieved with the modulator used for isolation of a given peptide probe. The equivalency numbers for these reference modulators are bolded. | | | | | | | | | | | | |
| # See also Table 10. Negative values indicate that an increase in the concentration of a compound results in a reduction of the binding of the peptide | | | | | | | | | | | | |
| # probe as compared to the binding of the probe in buffer. These negative values are expressed as a percentage of the reduction by ICI 182,780, | | | | | | | | | | | | |
| # so ICI 182,780 was scored −100 by definition, and is also bolded. Results for αβII were zero in all cases. | | | | | | | | | | | | |

^{b}EC50 is defined as the concentration in nanomolar of a given compound required to achieve fifty percent of the maximal signal for that compound.

TABLE 101

SEQ ID NO:

Class I

| | | |
|---|---|---|
| ER4 | SSNHQSRLIELLSR | 264 |
| D2 | GSEPKSRLLELLSAPVTDV | 222 |
| D30 | HPTHSSRLWELLMEATPTM | 223 |
| D11 | VESGSSRLMQLLMANDLLT | 224 |

Class II

| | | |
|---|---|---|
| D47 | HVYQHPLLLSLLSSEHESG | 225 |
| C33 | HVEMHPLLMGLLMESQWGA | 226 |
| D14 | QEAHGPLLWNLLSRSDTDW | 227 |

Class III

| | | |
|---|---|---|
| F6 | GHEPLTLLERLLMDDKQAV | 228 |
| D22 | LPYEGSLLLKLLRAPVEEV | 229 |
| D48 | SGWENSILYSLLSDRVSLD | 230 |
| D43 | AHGESSLLAWLLSGEYSSA | 231 |
| D17 | GVFCDSILCQLLAHDNARL | 232 |
| D41 | HHNGHSILYGLLAGSDAPS | 233 |
| D26 | LGERASLLDMLLRQENPAW | 234 |
| D40 | SGWNESTLYRLLQADAFDV | 235 |
| D15 | PSGGSSVLEYLLTHDTSIL | 236 |
| F4 | PVGEPGLLWRLLSAPVERE | 237 |

Misc.

| | | | |
|---|---|---|---|
| | D10 | WEEHSQMLLHLLDTGEAVW6 | 238 |
| ERβsp. | #293 | SSIKDFPNLISLLSR | 239 |
| GRIP-1 | NR1 | DSKGQTKLLQLLTTKSDQM | 240 |
| | NR2 | LKEKHKILHQLLQDSSSPV | 241 |
| | NR3 | KKKENALLRYLLDKDDTKD | 242 |
| SRC-1 | NR1 | YSQTSHKLVKLLTTTAEQQ | 243 |
| | NR2 | LTARHKILHRLLQEGSPSD | 244 |
| | NR3 | ESKDHQLLRYLLDKDEKDL | 245 |

TABLE 501

Peptides with Fold Induction of 2 or more

Fold Induction

| | | | |
|---|---|---|---|
| B2G1 | EFFRLRRLDRLLQDSFLLDLQPS* | (SEQ ID NO:61) | 12 |
| B1A1 | EFCPVGLLVHLLMQ* | (SEQ ID NO:62) | 11 |
| B1G8 | EFTSVSRLVTLLLQ* | (SEQ ID NO:63) | 6 |
| B4F6 | EFSGVPILHMLLMLPSSLDLQPS* | (SEQ ID NO:64) | 5 |
| B5H10 | EFSPPSSLLALLLGGKSLEPLPIRYKNVKRQFTSNSRGSVDLQPS* | (SEQ ID NO:65) | 4.5 |
| B6A1 | EFTGSRLLLKLLRFPDSSTCSQA | (SEQ ID NO:66) | 3.5 |
| B3E2 | EFGGSVLLRELLCCYDALEPTTTR | (SEQ ID NO:67) | 3.5 |
| B6F3 | EFFRATHLLRLLRTDSALDLQPS* | (SEQ ID NO:68) | 3 |
| B5A2 | EFGCSAILRYLLRSPRDLDLQPS* | (SEQ ID NO:69) | 3 |
| B6C4 | EFDRSSILVSLLSMVETLDLQPS* | (SEQ ID NO:70) | 2.5 |

TABLE 502A
AR NNK Sequences

| | |
|---|---|
| B1A4 | EFAWASVMLALEGG*WVLDLOPS* (SEQ ID NO:71) |
| B1B7 | EFCGELELLWEVFMLESLDLOPS* (SEQ ID NO:72) |
| 84C8 | EFLWEQIVVLLGWADCMLDLOPS* (SEQ ID NO:73) |
| B2A7 | EFPELLAMTRWGRHAALLEPQRLPPPRTTTQPQTEFERMFFFTR |
| | PMRTTGLLDLOPS* (SEQ ID NO:246) |
| B5G11 | EFRSSVFEQMYLCTGGSLDLOPS* (SEQ ID NO:247) |
| B8E9 | EFQQCMCAEVKSWLGGSLDLOPS* (SEQ ID NO:248) |
| B8H3 | EFHSRLRVEVVSWGIGSTCSQANSGRISYDL* (SEQ ID NO:249) |
| B8A10 | EFVNWDAVVPWSELVALLDLOPS (SEQ ID NO:250) |
| B18C4 | EFDSPWVWFGGEPGLNLLDLOPS (SEQ ID NO:251) |
| B22F6 | EFLSGLEMEVVLWHYGRLDLQPS (SEQ ID NO:252) |
| B23H12 | EFPELLAMTRWGRHAALLEPQRLPPPRTTTQPQTEFERMFFFTR |
| | PMRTTGLLDLQPS (SEQ ID NO:253) |
| M2H3 | EFRQSFVSEILGGGWLPLDLOPS (SEQ ID NO:254) |
| M4D1 | EFRFPFHEMVREWESMGLERVRYAEP (SEQ ID NO:255) |
| M7B1 | EFVGWFTGMAACSYAPDLDLOPS (SEQ ID NO:256) |
| D30 | HPTE-ISSRLWELLMEATPTM (SEQ ID NO:257) |

TABLE 502B
AR NNK Sequence Similarity

| | |
|---|---|
| HDAC5 | LAGGAVVLALEGG (SEQ ID NO:258) |
| B1A4 | EFAWASVMLALEGG*WVLDLQPS* (SEQ ID NO:259) |
| TR1P12 | CANVKQWKGGPVKIDP (SEQ ID NO:260) |
| B8E9 | EFQQCMCAEVKSWLGGSLDLQPS* (SEQ ID NO:261) |
| B8H3 | EFHSRLRVEVVSWGIGSTCSQANSGMSYDL* (SEQ ID |
| | NO:262) |
| Hsp27 | |
| | RLPEWSQWLGGS (SEQ ID NO:263) |

| Peptide Name | Fold induction |
|---|---|
| B2A7 | 5 |
| B5G11 | 6 |
| B8E9 | 4 |
| B8A10 | 1 |
| B8H3 | 22 |
| D17F5 | 2 |
| D18C4 | 1 |
| D22F6 | 10 |
| D23H12 | 6 |
| M2H3 | 10 |
| M4D1 | >2 |
| M7B10 | >2 |
| M9H11 | 1 |

TABLE 503
Yeast Specificity Graph (FIG. 3)

| Peptide | | | | | | |
|---|---|---|---|---|---|---|
| Name | No Lig | DHT | Test | MPA | CPA | RU |
| B2A7 | 0.371114 | 1.719086 | | | 0.291907 | 0.5 |
| B5G11 | 0.638157 | 3.963812 | | | 0.972635 | 0.9 |
| B8E9 | 0.498948 | 1.749957 | | | 0.563471 | 0.8 |
| B8A10 | 0.875262 | 0.616252 | | | 0.395473 | 0.4 |
| B8H3 | 0.173732 | 3.807301 | | | 0.392702 | −0.0 |
| D17F5 | 0.569453 | 1.196329 | 0.993155 | 2.209089 | 1.059607 | 1.2 |
| D18C4 | 1.115229 | 1.434154 | 0.969350 | 1.439357 | 1.533536 | 1.5 |
| D22F6 | 0.794349 | 7.846085 | 7.770933 | 4.556618 | 1.231158 | 1.2 |
| D23H12 | 0.845064 | 4.739387 | 2.440996 | 1.314433 | 0.426493 | 0.6 |
| M2H3 | 2.530208 | 24.119020 | 26.57396 | 19.24674 | 8.591983 | 6.8 |
| M4D1 | 0 | 0.394832 | 0.133723 | 1.697771 | −0.039550 | 0.1 |
| M7B10 | 0 | 3.933398 | 4.497615 | 6.903812 | 1.686521 | 0.1 |
| M9H11 | 1.402992 | 2.013497 | 1.558964 | 2.355091 | 1.928674 | 2.0 |

TABLE 504
Mammalian Cell Specificity Graph (FIG. 4)

| Peptide | no | | | | |
|---|---|---|---|---|---|
| Name | compound | DHT | MPA | CYP. | RU486 |
| D30/1269 | 0.038537 | 12.58706 | 15.61816 | 11.18426 | 0.846051 |
| 5G11 | 0.050283 | 15.83607 | 8.028699 | 4.462354 | 0.105076 |
| B8H3 | 0.131571 | 11.59878 | 11.33148 | 10.46618 | 3.878609 |
| B8E9 | 0.209111 | 19.63823 | 18.50569 | 16.01674 | 10.1916 |

(Units are Relative Light Units)

### ADDITIONAL REFERENCES

Anzick, S. L., Kononen, J., Walker, R. L., Azorsa, D. O., Tanner, M. M., Guan, X.-Y., Sauter, G., Kallioniemi, O.-P., Trent, J. M., and Meltzer, P. S. (1997) AIB1 a steroid receptor coactivator amplified in breast and ovarian cancer. Science 277, 965-968.

Chambraud, B., Berry, M., Redeuilh, G., Chambon, P., and Baulieu, E., (1990) Several regions of the human estrogen receptor are involved in the formation of receptor-heatshock protein 90 complexes. J. Biol. Chem. 265, 20686-20691.

Heery, D. M., Kalkhoven, E., Hoare, S., and Parker, M. G., (1997) A signature motif in transcriptional co-activators mediates binding to nuclear receptors. Nature, 387 733-736.

Kraus, W. L., McInerney, E. M., and Katzenellenbogen, B. S., (1995) Ligand-dependent, transcriptionally productive association of the amino-and carboxyl-terminal regions of a steroid hormone nuclear receptor. Proc. Natl. Acad. Sci., USA 92 12314-12318.

Montano, M. M., Muller, V., Trobaugh, A., and Katzenellenbogen, B. S., (1995) The carboxy-terminal F domain of the human estrogen receptor: role in the transcriptional activity of the receptor and the effectiveness of antiestrogens as estrogen antagonists. Mol Endocrinol., 9814-825.

Paech, K., Webb, P., Kuiper, G. G. J. M., Nilsson, S., Gustafsson, J.-A., Kushner, P. J., and Scanlan, T. S. (1997) Differential ligand activation of estrogen receptors ER α and ER β at AP1 sites. Science 277, 1508-1510.

1. In a method of identifying a binding peptide which binds a receptor, where said binding peptide is a member of a combinatorial library of peptides and said library is screened for the ability of its members to bind said receptor, the improvement wherein
said receptor is a surface or intracellular receptor of a cell,
said library is expressed in a plurality of cells, each cell coexpressing said receptor, or a ligand-binding receptor moiety thereof, and one member of said library, said cells collectively expressing all members of said library,
each cell further providing a signal producing system operably associated with said receptor or moiety such that a signal is produced which is indicative of whether said member binds said receptor or moiety in or on said cell,
said cells, when screened, are not integrated into a whole multicellular organism or a tissue or organ of such an organism,
where said peptides of said library are screened in a first screening when said receptor is in a first conformation, and one or more of the peptides of said library are screened in a second screening for binding to said receptor in a second and different conformation, said second screening being simultaneous with or subsequent to said first screening,
whereby peptides whose binding to the receptor is receptor conformation-sensitive are identified.

2. The method of claim 1 where said receptor is an intracellular receptor.

3. The method of claim 1 where said receptor is a nuclear receptor.

4. The method of claim 3 where said receptor is an estrogen receptor.

5. The method of claim 3 where said receptor is an androgen receptor.

6. The method of any of claims 1-5 where said cells are eukaryotic cells.

7. The method of claim 6 where said cells are mammalian cells.

8. The method of claim 6 where said cells are yeast cells.

9. The method of any of claims 1-8 where said receptor is a vertebrate receptor.

10. The method of claim 9 where said receptor is a mammalian receptor.

11. The method of claim 10 where said receptor is a human receptor.

12. The method of any of claims 1-11 where said signal producing system is endogenous to the cell.

13. The method of any of claims 1-11 where said signal producing system is exogenous to the cell.

14. The method of any of claims 1-13 where said signal producing system comprises a receptor-bound component which is fused to said receptor or moiety so as to provide a chimeric receptor.

15. The method of any of claims 1-14 where said signal producing system comprises a peptide-bound component which is fused to said peptide so as to provide a chimeric peptide.

16. The method of claim 14 where said signal producing system further comprises a peptide-bound component which is fused to said peptide so as to provide a chimeric peptide, whereby a signal is produced when the peptide-bound and receptor-bound components are brought into physical proximity as a result of the binding of the peptide to the receptor.

17. The method of claim 16 in which the cell is a mammalian cell.

18. The method of claim 16 in which the cell is a yeast cell.

19. The method of claim 16 where one of said components is a DNA-binding domain and another of said components is a complementary transactivation domain, and the signal producing system further comprises a reporter gene operably linked to an operator bound by said DNA-binding domain, the binding of the peptide to the receptor resulting in the constitution of a functional transactivation activator protein which activates expression of said reporter gene.

20. The method of claim 19 in which the domains are substantially identical to the DNA-binding and transactivation domains of a single naturally occurring transcriptional activator protein.

21. The method of claim 19 where the DNA-binding domain is selected from the group consisting of Gal4 and LexA.

22. The method of claim 19 where the transactivation domain is selected from the group consisting of E. coli B42, Gal4 activation domain II, and HSV VP16.

23. The method of claim 16 where one of said components is an amino terminal moiety of a reporter enzyme and another of said components is a carboxy terminal moiety of said enzyme, the binding of said peptide to the receptor resulting in the constitution from said moieties of a functional reporter enzyme.

24. The method of claim 23 where the enzyme is selected from the group consisting of DHFR, luciferase, chloramphenicol acetyltransferase, beta-lactamase, adenylate cyclase, and beta galactosidase.

25. The method of any of claims 1-24 where said screening is carried out in the presence of a known agonist of said receptor.

26. The method of any of claims 1-24 where said screening is carried out in the presence of a known antagonist of said receptor.

27. A method of predicting the receptor-modulating activity of a compound which modulates the biological activity of a receptor which comprises:
(I) identifying peptides which bind said receptor by the method of any of claims 1-26, said peptides differing in their ability to bind to said receptor depending on which of a plurality of different reference conformations the receptor is in, and
(II) using a plurality of said peptides to predict the receptor-modulating activity of a compound, by
(a) providing a panel comprising a plurality of members, said members including peptides identified in (I) above, said members differing in their ability to bind to said receptor depending on which of a plurality of different reference conformations the receptor is in, where the effect of a plurality of reference substances, known to modulate the biological activity of the receptor, on the binding of each member of the panel is known, and is characterized as a reference fingerprint for each such reference substance;
(b) screening a test substance of unknown activity relative to said receptor to determine its effect on the binding of each member of said panel to said receptor, thereby obtaining a test fingerprint for said test substance,
(c) comparing the test fingerprint to the reference fingerprints, and
(d) predicting the biological activity of the test substance, based on the assumption that its biological activity will be similar to that of reference substances with similar fingerprints.

28. The method of claim 27 where the effect of reference substances on the binding by said panel members is determined by
(a) providing a panel comprising a plurality of members, said members differing in their ability to bind to said receptor depending on which of a plurality of different reference conformations the receptor is in, and
(b) screening a plurality of reference substances known to modulate the biological activity of said receptor to determine their effect on the binding of each member of said panel to said receptor, thereby obtaining a reference fingerprint for each reference substance, said fingerprint comprising a plurality of panel-based descriptors, each panel-based descriptor characterizing the effect of the reference substance on the binding of a particular panel member to said receptor, said reference fingerprint's panel based descriptors collectively characterizing the effect of the reference substance on the binding of all of the panel members, individually, to said receptor.

29. The method of claim 28 where said panel members are obtained by a method which comprises:
(a) providing one or more ligands for the receptor;
(b) screening a first combinatorial library comprising a plurality of members for the ability to bind to a receptor in at least two different reference conformations, including at least one ligand-bound conformation, and
(c) based on said screening, providing a panel of first library members, said panel comprising members which differ with respect to their ability to binding to the receptor, depending on its conformation.

30. The method of claim 27 in which at least one reference conformation is an unliganded conformation of the receptor.

31. The method of claim 29 in which said panel comprises at least two of (i), (ii) and (iii) below:
(i) at least one member which binds the ligand-bound receptor more strongly than it binds the unliganded receptor, and which detectably binds the unliganded receptor,
(ii) at least one member which binds the ligand-bound receptor less strongly than it binds the unliganded receptor, and
(iii) at least one member which binds the ligand-bound receptor about as strongly as it binds the unliganded receptor, and detectably binds both.

32. The method of claim 1 wherein a plurality of different ligands are used in characterizing the panel.

33. The method of claim 27 in which the biological activity of the reference substances at said receptor is known for a plurality of different tissues, so that the biological activity of the test substance in said tissues is predicted.

34. The method of claim 27 in which the receptor is a nuclear receptor.

35. The method of claim 27 in which the receptor is an estrogen receptor (ER).

36. The method of claim 27 in which the receptor is an androgen receptor.

37. The method of claim 1 where said screenings are carried out on the same peptide library using the same receptor but in a plurality of different receptor conformations.

38. The method of claim 1 in which one of said conformations is an unliganded conformation.

39. The method of claim 38 in which another of said conformations is a liganded conformation.

40. The method of claim 1 in which one of said conformations is a liganded conformation.

41. The method of claim 40 in which one of said conformations is an agonist-liganded conformation.

42. The method of claim 40 in which one of said conformations is an antagonist-liganded conformation.

43. The method of claim 41 in which another of said conformations is an antagonist-liganded conformation.

44. The method of claim 1 in which, in the second screening, only peptides which bound the receptor in the first conformation are screened.

45. The method of claim 1 in which, in the second screening, only peptides which did not bind the receptor in the first conformation are screened.

46. The method of claim 40 in which said ligand is an exogenously added ligand.

47. The method of claim 40 in which said receptor is a nuclear receptor.

48. The method of claim 47 in which said receptor is an estrogen receptor.

49. The method of claim 47 in which said receptor is an androgen receptor.

50. The method of claim 8 where said signal producing system comprises a receptor-bound component which is fused to said receptor or moiety so as to provide a chimeric receptor where said signal producing system further comprises a peptide-bound component which is fused to said peptide so as to provide a chimeric peptide, whereby a signal is produced when the peptide-bound and receptor-bound components are brought into physical proximity as a result of the binding of the peptide to the receptor,
in which the yeast cells are obtained by mating haploid cells of a first mating type which express the peptide-bound component and haploid cells of a different mating type which express the receptor-bound component.

51. The method of claim 27 in which step (b) is performed in vitro.

52. The method of claim 27 in which step (b) is performed in a cell-based assay.

53. The method of claim 1 in which at least one of the receptor conformations is a ligand-bound conformation.

54. The method of claim 53 in which the ligand is exogenously added to the cell, and thereafter binds to the receptor to produce said ligand-bound conformation.

55. The method of claim 53 in which the ligand is a peptide coexpressed by said cell.
